(19) United States
(12) Patent Application Publication
(10) Pub. No.: US 2025/0375457 A1
(43) Pub. Date: Dec. 11, 2025

(54) TOPICAL PHARMACEUTICAL COMPOSITIONS AND USES THEREOF

(71) Applicant: Shenzhen Pharmacin Co., Ltd., Shenzhen (CN)

(72) Inventors: Shun CHEN, Shenzhen (CN); Zeren WANG, Shenzhen (CN); Peter FARINA, North Salem, NY (US); Lian WANG, Shenzhen (CN); Xiaoyan ZHOU, Shenzhen (CN); Jing SHAO, Shenzhen (CN)

(21) Appl. No.: 19/241,890

(22) Filed: Jun. 18, 2025

Related U.S. Application Data
(63) Continuation of application No. PCT/CN2023/139507, filed on Dec. 18, 2023.

(30) Foreign Application Priority Data
Dec. 19, 2022 (WO) PCT/CN2022/139952

(51) Int. Cl. A61K 31/553; A61K 9/00; A61K 9/06; A61K 47/10; A61K 47/14; A61K 47/18; A61K 47/20; A61K 47/26; A61K 47/32; A61P 17/00

(52) U.S. Cl. CPC A61K 31/553; A61K 9/0014; A61K 9/06; A61K 47/10; A61K 47/14; A61K 47/18; A61K 47/20; A61K 47/26; A61K 47/32; A61P 17/00

(57) ABSTRACT

Provided herein are methods of treating a skin-related disease or disorder (such as melasma). Also provided are pharmaceutical compositions of midostaurin or a pharmaceutically acceptable salt thereof formulated for topical administration. Further described are methods for preparing such topical pharmaceutical compositions and methods of using the same for treating a subject.

### CROSS REFERENCE TO RELATED APPLICATION

This application is a continuation of International Patent Application No. PCT/CN2023/139507, filed Dec. 18, 2023, which claims priority to International Patent Application No. PCT/CN2022/139952, filed on Dec. 19, 2022. which is incorporated herein by reference in its entirety.

### BACKGROUND

Skin hyperpigmentation conditions are characterized by general or localized darkening of an individual's normal skin color. Skin hyperpigmentation conditions include melasma, post-inflammatory hyperpigmentation, posttraumatic pigmentation, discoid lupus erythematous, freckles, aging pigmentation, plaques gravidarum, nevus of Ota and others. The hyperpigmentation can appear on all parts of the body. Currently, therapies for skin hyperpigmentation use non-selective toxins that reduce tyrosinase activity, including hydroquinone, azelaic acid, and kojic acid. These therapies are only partially effective and toxic. sometimes causing loss of skin pigmentation or ochronosis. Thus, there is a need for novel methods or products to treat hyperpigmentation disorders and limit serious side effects.

### INCORPORATION BY REFERENCE

All publications and patent applications mentioned in this specification are herein incorporated by reference to the same extent as if each individual publication or patent application was specifically and individually indicated to be incorporated by reference.

### SUMMARY

In one aspect, disclosed herein is a method of treating a skin-related disease or disorder, comprising topically applying a multi-targeting protein kinase inhibitor (e.g., midostaurin) or a salt thereof to a skin of a subject, wherein the skin is associated with a skin-related disease or disorder. In some embodiments, the skin-related disease or disorder is skin pigmentation disorder. In one aspect, disclosed herein is a method of reducing skin pigmentation, comprising topically applying a multi-targeting protein kinase inhibitor (e.g., midostaurin) or a salt thereof to a pigmented skin of a subject. In one aspect, disclosed herein is a method of reducing tyrosinase activity, comprising topically applying a multi-targeting protein kinase inhibitor (e.g., midostaurin) or a salt thereof to a skin of a subject in need thereof. In some embodiments, the subject has a skin hyperpigmentation condition. In some embodiments, the skin hyperpigmentation condition comprises a sunspot or an age spot. In some embodiments, the skin hyperpigmentation condition comprises melasma, post-inflammatory hyperpigmentation, posttraumatic pigmentation, discoid lupus erythematous, freckles, plaques gravidarum, nevus of Ota, or aging pigmentation. In one aspect, disclosed herein is a method of treating melasma, comprising topically applying a multi-targeting protein kinase inhibitor (e.g., midostaurin) or a salt thereof to a skin of a subject in need thereof. In some embodiments, the skin is on the subject's face. In some embodiments, the melasma is chloasma. In some embodiments, the melasma is caused by or exacerbated by one or more of: birth control pills, pregnancy, hormone therapy, stress, thyroid discase, sunlight exposure, inflammation, familial predisposition, or free radicals. In some embodiments, the treating comprises reducing the size of abnormal skin pigmentation, reducing the intensity of abnormal skin pigmentation, and/or eliminating the abnormal skin pigmentation associated with the melasma. In some embodiments, the method comprises topically applying a pharmaceutical composition, wherein the pharmaceutical composition comprises midostaurin or pharmaceutically acceptable salt thereof. In some embodiments, the treating comprises reducing melanin amount in an area of the skin by at least 25% compared to a similar area of skin in the same subject without the treatment. In some embodiments, the treating comprises reducing melanin amount in an area of the skin by at least 1.0 according to Melanin Distribution Score compared to a similar area of skin in the same subject without the treatment. In some embodiments, the treating comprises reducing melanin amount in an area of the skin by at least 1.0 according to Fontana-Masson staining scoring standard compared to a similar area of skin in the same subject without the treatment. In some embodiments, the multi-targeting protein kinase inhibitor or a salt thereof is midostaurin or a salt thereof. In some embodiments, the midostaurin or a salt thereof is administered in a therapeutically effective amount. In some embodiments, a therapeutically effective amount of the multi-targeting protein kinase inhibitor or a salt thereof is a dose that is 80% less than that for hydroquinone.

In one aspect. disclosed herein is a topical pharmaceutical composition. comprising a multi-targeting protein kinase inhibitor (optionally midostaurin) or a pharmaceutically acceptable salt thereof in an amount of 0.001% to about 20% wt. a carrier vehicle. and an excipient. wherein the topical pharmaceutical composition is formulated for topical administration. In some embodiments, the midostaurin or the pharmaceutically acceptable salt thereof is present in the composition in an amount of about 0.01 wt % to about 2 wt %. In some embodiments. the midostaurin or the pharmaceutically acceptable salt thereof is present in the composition in an amount of about 0.01 wt % to about 0.25 wt %. In some embodiments, the midostaurin or the pharmaceutically acceptable salt thereof is present in the composition in an amount of about 0.25 wt % to about 1.5 wt %. In some embodiments. the midostaurin or the pharmaceutically acceptable salt thereof is present in the composition in an amount of about 1.5 wt % to about 2.5 wt %. In some embodiments. the midostaurin or the pharmaceutically acceptable salt thereof is present in the composition in an amount of about 1.0 wt %. In some embodiments. the multi-targeting protein kinase inhibitor or the pharmaceutically acceptable salt thereof is midostaurin. In some embodiments. the topical pharmaceutical composition does not comprise a phospholipid. In some embodiments. the topical pharmaceutical composition comprises a phospholipid. In some embodiments. the carrier vehicle comprises an aqueous carrier vehicle. In some embodiments, the carrier vehicle comprises water. In some embodiments, the carrier vehicle comprises a non-aqueous carrier vehicle. In some embodiments, the carrier vehicle comprises polyethylene glycol (PEG), propylene glycol, glycerin (or glycerol), dimethyl sulfoxide (DMSO), petrolatum, mineral oil, wax, liquid paraffin, petroleum jelly, or a combination thereof. In some embodiments, the carrier vehicle comprises water, polyethylene glycol, propylene glycol, or glycerin, or a combination thereof. In some embodiments, the carrier vehicle is present in the composition in an amount of 40 wt % to 99 wt %. In some embodiments, the carrier vehicle is present in the composition in an amount of 70 wt % to 90 wt %. In some embodiments, the excipient comprises a surfactant, a penetration enhancer, an antioxidant, a sun-screening agent, a viscosity modifier, a pH stabilizer, or a moisturizer, or any combination thereof. In some embodiments, the excipient comprises a surfactant. In some embodiments, surfactant comprises a non-ionic surfactant. In some embodiments, the surfactant comprises glyceryl monooleate, glyceryl monolinoleate, polyethylene glycol-hydroxystearate (e.g., macrogol-15-hydroxystearate), polyoxyethylene sorbitan monooleate (e.g., polyoxyethylene (20) sorbitan monooleate, Tween 80, or polysorbate 80), polyoxylglycerides (e.g., caprylocaproyl polyoxyl-8 glycerides, oleoyl polyoxyl-6 glycerides), glyceryl polyethylene glycol ricinoleate (e.g., polyoxyl 35 hydrogenated castor oil), PEG palmitostearate (e.g., PEG-6 palmitostearate, PEG-32 palmitostearate), ethylene glycol palmitostearate, glycerol monostearate (or glyceryl monostearate, GMS), polyoxyethylene stearate (or macrogol stearate), sorbitan monostearate, polyglyceryl-3 dioleate. Sodium dodecyl sulfate (SDS), or polyoxyethylene alkyl ethers (e.g., Steareth-20), Steareth-2), or a combination thereof. In some embodiments, surfactant comprises an ionic surfactant (e.g., sodium dodecyl sulfate or SDS). In some embodiments, the surfactant comprises an emulsifier. In some embodiments, the emulsifier comprises polyoxyethylene sorbitan monooleate (e.g., polyoxyethylene (20) sorbitan monooleate, Tween 80, or polysorbate 80), PEG palmitostearate (e.g., PEG-6 palmitostearate, PEG-32 palmitostearate), ethylene glycol palmitostearate, glycerol monostearate (or glyceryl monostearate, GMS), polyoxyethylene stearate (or macrogol stearate), sorbitan monostearate, sodium dodecyl sulfate (SDS), oleoyl polyoxyl-6 glycerides), or polyoxyethylene alkyl ethers (e.g., Steareth-20, Steareth-2), or a combination thereof. In some embodiments, the topical pharmaceutical composition comprises two, three, four, or more surfactants. In some embodiments, the surfactant is present in the composition in an amount of about 1 wt % to 50 wt %. In some embodiments, the emulsifier is present in the composition in an amount of about 1 wt % to 50 wt %. In some embodiments, the topical pharmaceutical composition comprises a penetration enhancer. In some embodiments, the penetration enhancer comprises polyglyceryl-3 dioleate, diethylene glycol monoethyl ether, oleoyl polyoxyl-6 glycerides), 1-dodecylazacycloheptan-2-one (or Azone), or a combination thereof. In some embodiments, the penetration enhancer is present in the composition in an amount of about 0.1 wt % to 40) wt %. In some embodiments, the topical pharmaceutical composition comprises an antioxidant. In some embodiments, the antioxidant comprises butylated hydroxytoluene (BHT), butylated hydroxyanisole (BHA), ascorbyl palmitate, a-tocopherol acetate, acetone sodium bisulfite, acetylcysteine, ascorbic acid, vitamin E, ascorbyl palmitate, cysteine, cysteine hydrochloride, dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea, or tocopherols (e.g., d-a-tocopherol), or a combination thereof. In some embodiments, the antioxidant is present in the composition in an amount of about 0.001 wt % to 5 wt %. In some embodiments, the topical pharmaceutical composition comprises a sun-screening agent. In some embodiments, the sun-screening agent comprises avobenzone, bemotrizinol, benzophenone-3 (BZ-3, oxy benzone), bisoctizole, homosalate, octinoxate, octisalate, octocrylene, oxybenzone, titanium dioxide, or zinc oxide, or a combination thereof. In some embodiments, the sun-screening agent is present in the composition in an amount of about 0.001 wt % to 2 wt %. In some embodiments, the topical pharmaceutical composition comprises a viscosity modifier. In some embodiments, the viscosity modifier comprises aluminum monostearate, bentonite, polyacrylic acid (or PAA) (e.g., crosslinked polyacrylic acid), octadecyl alcohol, glyceryl behenate, or a combination thereof. In some embodiments, the topical pharmaceutical composition comprises a pH stabilizer. In some embodiments, the pH stabilizer comprises citric acid, glycolic acid, lactic acid, sodium hydroxide, sodium bicarbonate. L-arginine, triethanolamine, or a combination thereof. In some embodiments, the pH stabilizer comprises a pH buffering agent. In some embodiments, the topical pharmaceutical composition has a pH of about 5 to 8. In some embodiments, the topical pharmaceutical composition has a pH in an range from about 6.5 to about 7.5. In some embodiments. the topical pharmaceutical composition has a pH of about 7.0. In some embodiments, topical pharmaceutical composition comprises the moisturizer. In some embodiments, the moisturizer comprises petrolatum, mineral oil, wax, liquid paraffin, petroleum jelly (e.g., white Vaseline), glycerol (or glycerin), propylene glycol, or a combination thereof. In some embodiments, the topical pharmaceutical composition comprises a multifunctional excipient, wherein the multifunctional excipient is simultaneously two or more of a surfactant, a penetration enhancer, a viscosity modifier, a carrier vehicle, and a moisturizer. In some embodiments, the multifunctional excipient is simultaneously a surfactant and a penetration enhancer. In some embodiments, the multifunctional excipient is oleoyl polyoxyl-6 glycerides or polyglyceryl-3 dioleate. In some embodiments, the multifunctional excipient is simultaneously a moisturizer and a carrier vehicle. In some embodiments, the multifunctional excipient is petrolatum, mineral oil, wax, liquid paraffin, petroleum jelly, propylene glycol, or glycerol. In some embodiments. the topical pharmaceutical composition is in a form selected from an ointment, a gel, a cream, a lotion, a solution, an emulsion, a paste, a patch, a wipe, a swab, and a pad. In some embodiments, the topical pharmaceutical composition is an ointment. In some embodiments, the topical pharmaceutical composition comprises a) midostaurin or a pharmaceutically acceptable salt thereof in an amount of about 0.01% to about 5% wt. b) a surfactant in an amount of about 5% to about 20% wt. c) a penetration enhancer in an amount of about 0.05% to about 20% wt. d) a carrier vehicle in an amount of about 70% to about 90% wt, c) a moisturizer in an amount of about 5% to about 25% wt, and e) an antioxidant in an amount of about 0.001% to 5% wt. In some embodiments, the topical pharmaceutical composition comprises a multifunctional excipient. wherein the multifunctional excipient is simultaneously two or more of a surfactant, a penetration enhancer, a carrier vehicle, and a moisturizer. In some embodiments, the topical pharmaceutical composition is an ointment comprising: midostaurin or a pharmaceutically acceptable salt thereof in an amount of 0.05% to about 2% wt; a surfactant, wherein the surfactant comprises glyceryl monolinoleate, glyceryl monooleate, polyoxylglycerides (e.g., caprylocaproyl polyoxyl-8 glycerides), glyceryl polyethylene glycol ricinoleate (e.g., ricinoleate 35). polyethylene glycol-hydroxystearate (e.g., macrogol-15-hydroxystearate), or a combination thereof; a penetration enhancer, wherein the penetration enhancer comprises polyglyceryl-3 dioleate, diethylene glycol monoethyl ether, 1-dodecylazacycloheptan-2-one (Azone), or a combination thereof; a carrier vehicle, wherein the carrier vehicle comprises polyethylene glycol (PEG), mineral oil, or a combination thereof; a moisturizer, wherein the moisturizer comprises glycerol or propylene glycol, or a combination thereof; and an antioxidant, wherein the antioxidant comprises butylated hydroxytoluene (BHT), butylated hydroxyanisole (BHA), ascorbyl palmitate, a-tocopherol acetate, acetone sodium bisulfite, acetylcysteine, ascorbic acid, vitamin E, ascorbyl palmitate, cysteine, cysteine hydrochloride, or a combination thereof. In some embodiments, the topical pharmaceutical composition comprises; midostaurin or a pharmaceutically acceptable salt thereof in an amount of about 0.5% to about 1.5% wt; a surfactant in an amount of about 7% to about 15% wt, wherein the surfactant comprises caprylocaproyl polyoxyl-8 glycerides; a penetration enhancer in an amount of about 0.5% to about 3% wt, wherein the penetration enhancer comprises 1-dodecylazacycloheptan-2-one; a carrier vehicle in an amount of about 70% to about 80% wt, wherein the carrier vehicle comprises a polyethylene glycol having a number average molecular weight of about 300 to 500 g/mol, a polyethylene glycol having a number average molecular weight of about 3000 to 5000 g/mol, or a combination thereof; a moisturizer in an amount of about 10% to about 15% wt, wherein the moisturizer comprises glycerol; and an antioxidant in an amount of about 0.001% to 1% wt, wherein the antioxidant comprises butylated hydroxytoluene (BHT), butylated hydroxyanisole (BHA), ascorbyl palmitate, a-tocopherol acetate, acetone sodium bisulfite, acetylcysteine, ascorbic acid, vitamin E, ascorbyl palmitate, cysteine, cysteine hydrochloride, or a combination thereof. In some embodiments, the topical pharmaceutical composition is a gel. In some embodiments, the topical pharmaceutical composition is a gel comprising; midostaurin or a pharmaceutically acceptable salt thereof in an amount of about 0.01% to about 5% wt; a surfactant in an amount of about 5% to about 50% wt; optionally, a penetration enhancer in an amount of about 1% to about 30% wt; a carrier vehicle in an amount of about 60% to about 95% wt; a viscosity modifier in an amount of about 0.01% to about 5% wt; optionally, a pH stabilizer; and an antioxidant in an amount of about 0.001% to 5% wt. In some embodiments, the topical pharmaceutical composition comprises a multifunctional excipient, wherein the multifunctional excipient is simultaneously a surfactant and a penetration enhancer. In some embodiments, the topical pharmaceutical composition is a gel comprising; a) midostaurin or a pharmaceutically acceptable salt thereof in an amount of 0.05% to about 2% wt; b) a surfactant, wherein the surfactant comprises glyceryl monolinoleate, glyceryl monooleate. polyoxylglycerides (e.g., caprylocaproyl polyoxyl-8 glycerides), glyceryl polyethylene glycol ricinoleate (e.g., macrogolglycerol ricinoleate 35), polyoxyethylene sorbitan monooleate (e.g., polysorbate 80), or a combination thereof; c) optionally, a penetration enhancer, wherein the penetration enhancer comprises polyglyceryl-3 dioleate, oleoyl polyoxyl-6 glycerides diethylene glycol monoethyl ether, 1-dodecylazacycloheptan-2-one (Azone); d) a viscosity modifier, wherein the viscosity modifier comprises polyacrylic acid; c) optionally, a pH stabilizer, wherein the pH stabilizer comprises sodium hydroxide, sodium bicarbonate, L-arginine, triethanolamine, or a combination thereof; f) a carrier vehicle, wherein the carrier vehicle comprises water; and g) an antioxidant, wherein the antioxidant comprises butylated hydroxytoluene (BHT), butylated hydroxyanisole (BHA), ascorbyl palmitate, a-tocopherol acetate. acetone sodium bisulfite, acetylcysteine, ascorbic acid, vitamin E, ascorbyl palmitate, cysteine, cysteine hydrochloride, or a combination thereof. In some embodiments, the topical pharmaceutical composition comprises; a) midostaurin or a pharmaceutically acceptable salt thereof in an amount of about 0.05% to about 1.5% wt; b) a surfactant in an amount of about 5% to about 20% wt, wherein the surfactant comprises caprylocaproyl polyoxyl-8 glycerides, polysorbate 80, or a combination thereof; c) optionally a penetration enhancer in an amount of about 5% to about 25% wt, wherein the penetration enhancer comprises diethylene glycol monoethyl ether; d) a viscosity modifier in an amount of about 0.1% to about 2% wt, wherein the viscosity modifier comprises crosslinked polyacrylic acid; c) optionally, a pH stabilizer, wherein the pH stabilizer comprises triethanolamine; f) an aqueous carrier vehicle in an amount of about 70% to about 90% wt, wherein the aqueous carrier vehicle comprises water; and g) an antioxidant in an amount of about 0.001% to 2% wt, wherein the antioxidant comprises butylated hydroxytoluene (BHT), butylated hydroxyanisole (BHA), ascorbyl palmitate, or a combination thereof. In some embodiments, the topical pharmaceutical composition has a pH of about 6.5 to about 7.5. In some embodiments, the topical pharmaceutical composition has a pH of about 7.0. In some embodiments, the topical pharmaceutical composition is a cream. In some embodiments, the topical pharmaceutical composition comprises; midostaurin or a pharmaceutically acceptable salt thereof in an amount of about 0.01% to about 5% wt; a carrier vehicle in an amount of about 30% to about 70% wt; a surfactant in an amount of about 5% to about 50% wt; optionally a moisturizer in an amount of about 5% to about 30% wt; optionally a penetration enhancer in an amount of about 1% to about 40% wt; optionally a viscosity modifier in an amount of about 0.01% to about 5% wt; and an antioxidant in an amount of about 0.001% to 5% wt. In some embodiments, the topical pharmaceutical composition comprises; midostaurin or a pharmaceutically acceptable salt thereof in an amount of 0.05% to about 5% wt; a carrier vehicle comprising water and optionally polyethylene glycol (PEG), propylene glycol, liquid paraffin, or petroleum jelly (e.g., white Vaseline), or a combination thereof; a surfactant, wherein the surfactant comprises glyceryl monooleate, glyceryl monolinoleate, polyoxyethylene sorbitan monooleate (e.g., polyoxyethylene (20) sorbitan monooleate, Tween 80, or polysorbate 80), PEG palmitostearate (e.g., PEG-6 palmitostearate, PEG-32 palmitostearate), ethylene glycol palmitostearate, glycerol monostearate (or glyceryl monostearate, GMS), polyoxyethylene stearate (or macrogol stearate), sorbitan monostearate, sodium dodecyl sulfate (SDS), polyoxyethylene alkyl ethers (e.g., Steareth-20, Steareth-2). or a combination thereof; optionally a moisturizer comprising glycerol or propylene glycol, or a combination thereof; optionally a penetration enhancer, wherein the penetration enhancer comprises oleoyl polyoxyl-6 glycerides, polyglyceryl-3 dioleate, diethylene glycol monoethyl ether, 1-dodecylazacycloheptan-2-one (or Azone), or a combination thereof; and an antioxidant, wherein the antioxidant comprises butylated hydroxytoluene (BHT), butylated hydroxyanisole (BHA), ascorbyl palmitate, a-tocopherol acetate, acetone sodium bisulfite, acetylcysteine, ascorbic acid, vitamin E, ascorbyl palmitate, cysteine, cysteine hydrochloride, or a combination thereof. In some embodiments, the topical pharmaceutical composition comprises; midostaurin or a pharmaceutically acceptable salt thereof in an amount of about 0.1% to about 2% wt; a surfactant in an amount of about 5% to about 50% wt. wherein the surfactant comprises glyceryl monolinoleate, glyceryl monooleate, PEG palmitostearate (e.g., PEG-6 palmitostearate, PEG-32 palmitostearate), ethylene glycol palmitostearate, glycerol monostearate, polyoxylethylene stearate (e.g., macrogol-75 stearate), polyoxyethylene sorbitan monooleate (e.g., polysorbate 80)), sorbitan monostearate, sodium dodecyl sulfate, or a combination thereof; optionally a penetration enhancer in an amount of about 1% to about 35% wt, wherein the penetration enhancer comprises oleoyl polyoxyl-6 glycerides, polyglyceryl-3 dioleate, diethylene glycol monoethyl ether, 1-dodecylazacycloheptan-2-one (Azone), or a combination thereof; optionally a moisturizer in an amount of about 5% to about 25% wt, wherein the moisturizer comprises glycerol (glycerin), propylene glycol, or a combination thereof; an antioxidant in an amount of about 0.01% to about 2% wt, wherein the antioxidant comprises butylated hydroxytoluene (BHT), butylated hydroxyanisole (BHA), ascorbyl palmitate, a-tocopherol acetate, acetone sodium bisulfite, acetylcysteine, ascorbic acid, vitamin E, ascorbyl palmitate, cysteine, cysteine hydrochloride, or a combination thereof; and a carrier vehicle in an amount of about 30% to about 70% wt, wherein the carrier vehicle comprises water and optionally liquid paraffin, or white Vaseline. In some embodiments, the topical pharmaceutical composition comprises; midostaurin or a pharmaceutically acceptable salt thereof in an amount of about 0.5% to about 2% wt; a surfactant in an amount of about 5% to about 35% wt, wherein the surfactant comprises glyceryl monolinoleate or glyceryl monooleate. PEG palmitostearate (e.g., PEG-6 palmitostearate. PEG-32 palmitostearate), ethylene glycol palmitostearate, glycerol monostearate. polyoxylethylene stearate (e.g., macrogol-75 stearate), polyoxyethylene sorbitan monooleate (e.g., polysorbate 80), sorbitan monostearate, or a combination thereof; a penetration enhancer in an amount of about 1% to about 35% wt, wherein the penetration enhancer comprises polyglyceryl-3 dioleate. diethylene glycol monoethyl ether, 1-dodecylazacycloheptan-2-one (or Azone), or a combination thereof; an antioxidant in an amount of about 0.01% to about 0.1% wt, wherein the antioxidant comprises butylated hydroxytoluene (BHT), butylated hydroxyanisole (BHA), ascorbyl palmitate, or a combination thereof; and a carrier vehicle in an amount of about 60% to about 70% wt, wherein the carrier vehicle comprises water. In some embodiments, the topical pharmaceutical composition comprises; midostaurin or a pharmaceutically acceptable salt thereof in an amount of about 1% wt; glyceryl monooleate in an amount of about 10% wt; polyoxyethylene sorbitan monooleate (e.g., polysorbate 80) in an amount of about 5% to about 14% wt; sorbitan monostearate in an amount of about 3.5% wt; polyglyceryl-3 dioleate in an amount of about 5%, an antioxidant in an amount of about 0.06% wt, wherein the antioxidant comprises butylated hydroxytoluene (BHT), butylated hydroxyanisole (BHA) and ascorbyl palmitate; and water in an amount of about 66.5% wt. In some embodiments, the topical pharmaceutical composition further comprises a sun-screening agent. In some embodiments, the sun-screening agent comprises avobenzone, bemotrizinol, benzophenone-3 (BZ-3, oxy benzone), bisoctizole, homosalate, octinoxate, octisalate, octocrylene, oxybenzone, titanium dioxide, or zinc oxide, or a combination thereof. In some embodiments, the topical pharmaceutical composition reduces melanin amount in an area of skin by at least 25% compared to a similar area of skin in the same subject without any treatment, wherein the melanin amount is measured by a melanin distribution score using guinea pig as a model. In some embodiments, a reduction of melanin amount in an area of skin treated with the topical pharmaceutical composition is at least 100% relative to that of a similar area of skin in the same subject treated with a reference topical pharmaceutical composition comprising hydroquinone, wherein the topical pharmaceutical composition is administered at a dose that is lower than a dose of the reference topical pharmaceutical composition. In some embodiments, the topical pharmaceutical composition reduces tyrosinase activity in an area of skin by at least 25% compared to a similar area of skin in the same subject without any treatment, wherein the tyrosinase activity is measured per gram of tissue using guinea pig as a model. In some embodiments, a reduction of tyrosinase activity in an area of skin treated with the topical pharmaceutical composition is at least about 100% relative to that of a similar area of skin in the same subject treated with a reference topical pharmaceutical composition comprising hydroquinone, wherein the topical pharmaceutical composition is administered at a dose that is lower than a dose of the reference topical pharmaceutical composition. In some embodiments, a skin irritation level in an area of skin treated with the topical pharmaceutical composition is at most mild, and wherein the topical pharmaceutical composition comprises at most 1.0% wt midostaurin. In some embodiments, a skin irritation score in an area of skin treated with the topical pharmaceutical composition is at most about 50% relative to that of a similar area of skin in the same subject treated with a reference topical pharmaceutical composition comprising hydroquinone, wherein the topical pharmaceutical composition is administered at a dose that is lower than a dose of the reference topical pharmaceutical composition. In some embodiments, the topical pharmaceutical composition is chemically stable for at least 7 days under the condition of 4500 lux (lx) in light exposure or when it is stored at about 60° C. In some embodiments, the topical pharmaceutical composition retains at least 90 wt % of midostaurin or the pharmaceutically acceptable salt thereof when stored for 7 days under the condition of 4500 lux (lx) in light exposure and about 15° C. to about 25° C. in temperature. In some embodiments, the topical pharmaceutical composition retains at least 90 wt % of midostaurin or the pharmaceutically acceptable salt thereof when stored at about 60° C. for 7 days. In some embodiments, the topical pharmaceutical composition retains at least 90 wt % of midostaurin or the pharmaceutically acceptable salt thereof after at least one freeze-thaw cycle. In some embodiments, the topical pharmaceutical composition retains at least 90 wt % of midostaurin or the pharmaceutically acceptable salt thereof after stored at refrigerated conditions for at least 3, 6, 9, 12, 24, or 36 months. In some embodiments, the topical pharmaceutical composition retains at least 95 wt % of midostaurin or the pharmaceutically acceptable salt thereof after stored at refrigerated conditions for at least 3, 6, 9, 12, 24, or 36 months. In some embodiments, the topical pharmaceutical composition retains at least 90 wt % of midostaurin or the pharmaceutically acceptable salt thereof after stored at ambient conditions for at least 3, 6, 9, 12, or 24 months. In some embodiments, the topical pharmaceutical composition retains at least 95 wt % of midostaurin or the pharmaceutically acceptable salt thereof after stored at ambient conditions for at least 3, 6, 9, 12, or 24 months. In some embodiments, the topical pharmaceutical composition retains at least 90 wt % of midostaurin or the pharmaceutically acceptable salt thereof after stored at about 40° C. for at least 1, 3, or 6 months. In some embodiments, the topical pharmaceutical composition retains at least 95 wt % of midostaurin or the pharmaceutically acceptable salt thereof after stored at about 40° C. for at least 1, 3, or 6 months. In some embodiments, the amount of midostaurin or the pharmaceutically acceptable salt thereof is determined according to High-performance liquid chromatography (HPLC) assay (e.g., described in Table A-1). In some embodiments, the topical pharmaceutical composition contains no more than 5 wt % of total impurity after at stored at refrigerated conditions for at least 3, 6, 9, 12, 24, or 36 months. In some embodiments, the topical pharmaceutical composition contains no more than 2 wt % of total impurity after at stored at refrigerated conditions for at least 3, 6, 9, 12, 24, or 36 months. In some embodiments, the topical pharmaceutical composition contains no more than 5 wt % of total impurity after at stored at ambient conditions for at least 3, 6, 9, 12, or 24 months. In some embodiments, the topical pharmaceutical composition contains no more than 2 wt % of total impurity after at stored at ambient conditions for at least 3, 6, 9, 12, or 24 months. In some embodiments, the topical pharmaceutical composition contains no more than 5 wt % of total impurity after at stored at about 40° C. for at least 1, 3, or 6 months. In some embodiments, the topical pharmaceutical composition contains no more than 2 wt % of total impurity after at stored at about 40° C. for at least 1, 3, or 6 months. In some embodiments, the amount of total impurity is determined according to High-performance liquid chromatography (HPLC) impurity analysis (e.g., described in Table B-2).

Disclosed herein is a method of treating a skin-related disease or disorder, comprising topically applying a topical pharmaceutical composition to a skin of a subject in need thereof, wherein the topical pharmaceutical composition comprises midostaurin or a pharmaceutically acceptable salt thereof. In some embodiments, the skin-related disease or disorder comprises skin pigmentation disorders. In some embodiments, the method comprises applying the topical pharmaceutical composition described herein to a skin of a subject in need thereof.

Disclosed herein is a method of reducing skin pigmentation, comprising topically applying a topical pharmaceutical composition to a skin of a subject in need thereof, wherein the topical pharmaceutical composition comprises midostaurin or a pharmaceutically acceptable salt thereof. In some embodiments, the method comprises topically applying the topical pharmaceutical composition described herein to a skin of a subject in need thereof.

Disclosed herein is a method of reducing tyrosinase activity, comprising topically applying a topical pharmaceutical composition to a skin of a subject in need thereof, wherein the topical pharmaceutical composition comprises midostaurin or a pharmaceutically acceptable salt thereof. In some embodiments, the method comprises topically applying the topical pharmaceutical composition described herein to a skin of a subject in need thereof.

Disclosed herein is a kit comprising a package enclosing the topical pharmaceutical composition described herein.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which;

FIG. 1 shows the equilibrium solubility of midostaurin in different solvents.

FIG. 2 shows the solubility of midostaurin in various solubilizer-water mixture systems.

FIG. 3 illustrates different layers of human skin.

FIG. 4 illustrates the Franz diffusion cells system for conducting skin deposition test.

FIG. 5 shows the results for skin deposition of midostaurin and hydroquinone when prepared at various concentrations in a mixture of DMSO and propylene glycol.

FIG. 6 shows the results for skin deposition of midostaurin in gel formulations with different excipients.

FIGS. 7A-7D show the crystallization under polarization microscope for midostaurin gel formulations lot A1, lot A2, lot A3, and lot A4, respectively. The micrographs show needle-like and star-like crystallization of midostaurin in the gel formulation, especially in Lot A1 gel.

FIGS. 8A and 8B show the crystallization under polarization microscope for midostaurin cream formulation lot B1 and lot B2, respectively. The micrographs show needle-like and snowflake-like crystallization of midostaurin in the cream formulation.

FIG. 9 shows the results for skin deposition of 1% and 0.1% midostaurin cream and 2% hydroquinone cream, wherein one 2% hydroquinone cream is commercially available, and all other formulations are prepared according to components of lot B2.

FIGS. 10A-10D depict four ternary phase diagrams, each showing phase behavior of a system consisting of Azone, water, and a mixture of Tefose 63 and Labrafil M 1944CS at a certain ratio.

FIGS. 11A-11E shows the crystallization under polarization microscope for midostaurin cream formulation lot B3, lot B4, lot B5, lot B6, and lot B7, respectively. The micrographs of lots B3 and B7 shows that the droplets are evenly distributed, without needle like or star like crystallization. The micrographs of lots B4 and B5 show that the star-like crystallization occurred. The micrograph of lot B6 shows that the droplet distribution became wider, and the droplet diameter became larger.

FIG. 12 depicts a ternary phase diagram, showing phase behavior of a system consisting of Peceol, water, and Gelot 64.

FIGS. 13A-13B show the crystallization under polarization microscope for midostaurin cream formulation lot B8 and the blank carrier vehicle of lot B8, respectively. The micrograph of lot B8 is similar to that of blank carrier vehicle, indicating that it is in a stable state.

FIG. 14 shows the midostaurin deposition results for lots B9, B10, B11, and cream lot B2 used in pharmacodynamics (PD) efficacy experiment, comparing the effects of different penetration enhancers on skin deposition of midostaurin in cream formulations.

FIG. 15A shows a picture of a guinea pig with its back shaved and divided into six areas.

FIGS. 15B-C show pictures of a control group guinea pig (N=1) with no UV irritation.

FIGS. 16A-16B show two pictures of the back of a guinea pig, where the skin colors are different at the six areas receiving different treatments, which are: (i) top left area, no treatment (model); (ii) top right area, 2% hydroquinone cream; (iii) bottom left area, 2% hydroquinone solution; (iv) middle left area, 1% midostaurin cream; (v) middle right area, 0.1% midostaurin cream; and (vi) bottom right area 1% midostaurin solution, respectively. The type of treatments is annotated in FIG. 16A.

FIG. 17 shows the tyrosinase activity for seven different groups, control group (no UV irritation), model group (UV irritation followed by no treatment), 2% hydroquinone cream, 2% hydroquinone solution, 1% midostaurin cream, 0.1% midostaurin cream, and 1% midostaurin solution treatment groups.

FIG. 18 shows the melanin distribution score for seven different groups, control group (no UV irritation), model group (UV irritation followed by no treatment), 2% hydroquinone cream, 2% hydroquinone solution, 1% midostaurin cream, 0.1% midostaurin cream, and 1% midostaurin solution treatment groups.

FIG. 19A shows the melanin distribution score for seven different groups, control group (no UV irritation), model group (UV irritation followed by no treatment), 2% hydroquinone positive control (referred to as “2-1”), 0.01% midostaurin cream (referred to as “2-2”), 0.05% midostaurin cream (referred to as “2-3”), 0.25% midostaurin cream (referred to as “2-4”), 1.5% midostaurin cream (referred to as “2-5”).

FIG. 19B shows the tyrosinase activity for seven different groups, control group (no UV irritation), model group (UV irritation followed by no treatment), 2% hydroquinone positive control (referred to as “2-1”), 0.01% midostaurin cream (referred to as “2-2”), 0.05% midostaurin cream (referred to as “2-3”), 0.25% midostaurin cream (referred to as “2-4”), 1.5% midostaurin cream (referred to as “2-5”).

### DETAILED DESCRIPTION

The present disclosure is generally directed to topical pharmaceutical compositions comprising multi-targeting protein kinase inhibitor (e.g., midostaurin) or a pharmaceutically acceptable salt thereof. and methods of use and making thereof. A multi-targeting protein kinase inhibitor can target at least two protein kinases. A multi-targeting protein kinase inhibitor can inhibit two or more protein kinases selected from the group consisting of; PKC α/β/γ, Syk, Flk-1, Akt, PKA, c-Kit, c-Fgr, c-Src, FLT3, PDFRB, and VEGFR1/2. In some cases, the multi-targeting protein kinase inhibitor inhibits PKC α, PKC β, and PKCγ. In some cases, the multi-targeting protein kinase inhibits PKC α/β/γ and Syk. In some cases, the multi-targeting protein kinase inhibitor inhibits PKC α/β/γ and Akt. In some cases, the multi-targeting protein kinase inhibitor inhibits PKC α/β/γ and PKA. In some cases, the multi-targeting protein kinase targets PKC α/β/γ and c-Kit. In some cases, the multi-targeting protein kinase inhibitor inhibits PKC α/β/γ and c-Fgr. In some cases, the multi-targeting protein kinase inhibitor inhibits PKC α/β/γ and c-Src. In some cases, the multi-targeting protein kinase inhibitor inhibits PKC α/β/γ and FLT3. In some cases, the multi-targeting protein kinase inhibitor inhibits PKC α/β/γ and PDFRB. In some cases, the multi-targeting protein kinase inhibitor inhibits PKC α/β/γ and VEGFR1/2. In some cases, the multi-targeting protein kinase inhibitor inhibits PKC α/β/γ, Syk, Flk-1, Akt, PKA, c-Kit, c-Fgr, c-Src, FLT3, PDFRB, and VEGFR1/2. In some cases, the multi-targeting protein kinase inhibitor is midostaurin.

A topical pharmaceutical composition comprising multi-targeting protein kinase inhibitor (e.g., midostaurin) or a pharmaceutically acceptable salt thereof can comprise midostaurin or a salt thereof. Rydapt R (midostaurin) was approved by FDA in 2017 for the treatment of AML (acute myeloid leukemia) and ASM (aggressive systemic mastocytosis). Midostaurin is a multi-kinase inhibitor for oral use. In one aspect, the present disclosure discloses that topically administering midostaurin can reduce the skin hyperpigmentation in UV induced guinea pig model, in a dose-dependent manner. Without being bound by theory, it is discovered that midostaurin, when applied topically, is more effective and has better safety profile (e.g., better skin tolerance) compared to other treatments of skin pigmentation currently on the market.

Midostaurin is small molecule kinase inhibitor currently available for oral use. The molecular formula for midostaurin is C₃₅H₃₀N₄O₄. The molecular weight is 570.65 g/mol. The structural formula for midostaurin is as follows:

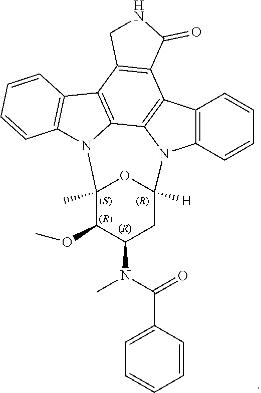

Midostaurin has a high log P value of 5.8 and is very hard to dissolve in aqueous solution. In addition, midostaurin is not stable in many conditions, such as acid, alkali, oxidation, thermal, photolytic degradation, which makes the development even more challenging. Thus, it can be very challenging to develop an appropriate topical formulation for midostaurin. Different excipients used in compositions of the topical formulation can affect the amount of the midostaurin delivered to different layers of the skin.

In one aspect, provided herein are midostaurin pharmaceutical compositions for topical administration. The midostaurin topical formulations are useful as therapeutics that alleviate, abate or eliminate one or more skin-related conditions in a subject in need thereof, as further described herein. In some embodiments, the skin-related conditions include skin hyperpigmentation conditions, for example, melasma, post-inflammatory hyperpigmentation, posttraumatic pigmentation, discoid lupus erythematous, freckles, plaques gravidarum, nevus of Ota, aging pigmentation and others.

In one aspect, the present embodiments provide novel methods or products to treat skin hyperpigmentation disorders. In some embodiments, the midostaurin used in the topical pharmaceutical compositions described herein is a midostaurin free base. In some embodiments, the midostaurin used in the formulations described herein is a midostaurin salt.

In one aspect, also disclosed herein are methods of using a metabolite of midostaurin. In some embodiments of methods disclosed herein, midostaurin or a salt thereof can be replaced by a metabolite of midostaurin. Exemplary metabolites of midostaurin include CGP 52421 and CGP 62221,

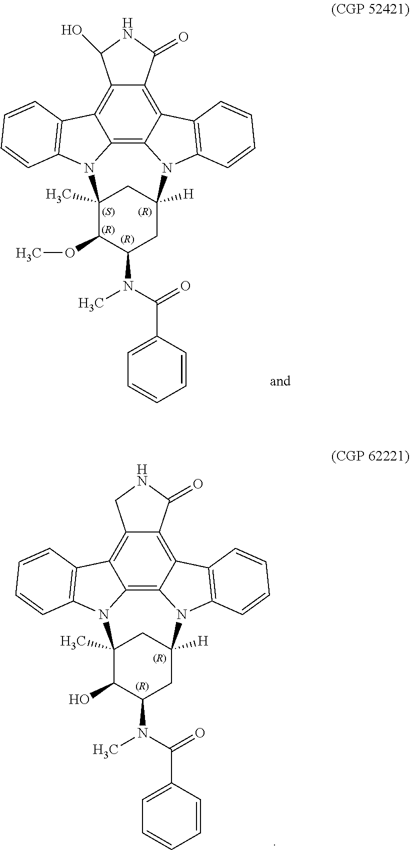

### Midostaurin Topical Pharmaceutical Compositions

In one aspect, disclosed herein are topical pharmaceutical compositions comprising midostaurin or a pharmaceutically acceptable salt thereof. The present topical formulations can include, but are not limited to, an ointment, a gel, a cream, a lotion, a solution, an emulsion, a paste, a patch, a wipe, a swab, a pad, and any other form suitable for topical delivery of midostaurin of a pharmaceutically acceptable salt thereof. It is envisioned that a lotion, a solution, an emulsion, a paste, are also included where certain components described herein are in a cream while other components are in a gel or an ointment.

In some embodiments, the topical pharmaceutical compositions also comprise a carrier vehicle. In some embodiments, the topical pharmaceutical compositions also comprise an excipient. In some embodiments, the topical pharmaceutical compositions are formulated for topical administration.

In some embodiments, the midostaurin is present in the pharmaceutical composition in the form of a free base. In some embodiments, midostaurin is present in the form of a pharmaceutically acceptable salt. As used herein, a pharmaceutically acceptable salt includes, but is not limited to, metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. Pharmaceutically acceptable salts include bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafuorobutyrate), bis(pentafluoropropionate), bis(pyridine carboxylate), bis(trifluoroacetate), chlorhydrate, and sulfate pentahydrate. Other representative pharmaceutically acceptable salts include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, 15ydroxyapat, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A hydrate is another example of a pharmaceutically acceptable salt. In some embodiments, the topical pharmaceutical composition comprises midostaurin.

Disclosed herein are topical pharmaceutical compositions comprising midostaurin or a pharmaceutically acceptable salt thereof. In some embodiments, midostaurin or the pharmaceutically acceptable salt thereof is present in the composition in an amount of 0.001% to about 30 wt %. In some embodiments, midostaurin or a pharmaceutically acceptable salt thereof is present in the composition in an amount of about 0.001 wt % to about 0.01 wt %, about 0.01 wt % to about 0.1 wt %, about 0.1 wt % to about 0.5 wt %, about 0.5 wt % to about 1 wt %, about 1 wt % to about 2 wt %, about 2 wt % to about 5 wt %, about 5 wt % to about 10 wt %, about 10 wt % to about 20 wt %, or about 20 wt % to about 30 wt %. In some embodiments, midostaurin or the pharmaceutically acceptable salt thereof is present in the topical pharmaceutical composition in an amount of about 0.001 wt % to about 0.01 wt %. In some embodiments, midostaurin or the pharmaceutically acceptable salt thereof is present in the topical pharmaceutical composition in an amount of about 0.01 wt % to about 0.1 wt %. In some embodiments, midostaurin or the pharmaceutically acceptable salt thereof is present in the topical pharmaceutical composition in an amount of about 0.1 wt % to about 0.5 wt %. In some embodiments, midostaurin or the pharmaceutically acceptable salt thereof is present in the topical pharmaceutical composition in an amount of about 0.5 wt % to about 1 wt %. In some embodiments, midostaurin or the pharmaceutically acceptable salt thereof is present in the topical pharmaceutical composition in an amount of about 1 wt % to about 2 wt %. In some embodiments, midostaurin or the pharmaceutically acceptable salt thereof is present in the topical pharmaceutical composition in an amount of about 2 wt % to about 5 wt %. In some embodiments, midostaurin or the pharmaceutically acceptable salt thereof is present in the topical pharmaceutical composition in an amount of about 5 wt % to about 10 wt %. In some embodiments, midostaurin or the pharmaceutically acceptable salt thereof is present in the topical pharmaceutical composition in an amount of about 10 wt % to about 20 wt %. In some embodiments, midostaurin or the pharmaceutically acceptable salt thereof is present in the topical pharmaceutical composition in an amount of about 20 wt % to about 30 wt %. In some embodiments, midostaurin or the pharmaceutically acceptable salt thereof is present in the topical pharmaceutical composition in an amount of about 0.001 wt % to about 1.0 wt %. In some embodiments, midostaurin or the pharmaceutically acceptable salt thereof is present in the topical pharmaceutical composition in an amount of about 0.01 wt % to about 1.0 wt %. In some embodiments, midostaurin or the pharmaceutically acceptable salt thereof is present in the topical pharmaceutical composition in an amount of about 0.01 wt % to about 2 wt %. In some embodiments, midostaurin or the pharmaceutically acceptable salt thereof is present in the topical pharmaceutical composition in an amount of about 0.01 wt % to about 0.25 wt %. In some embodiments, midostaurin or the pharmaceutically acceptable salt thereof is present in the topical pharmaceutical composition in an amount of about 0.25 wt % to about 1.5 wt %. In some embodiments, midostaurin or the pharmaceutically acceptable salt thereof is present in the topical pharmaceutical composition in an amount of about 0.1 wt % to about 1.0 wt %. In some embodiments, midostaurin or the pharmaceutically acceptable salt thereof is present in the topical pharmaceutical composition in an amount of about 0.25 wt % to about 0.75 wt %. In some embodiments, midostaurin or the pharmaceutically acceptable salt thereof is present in the topical pharmaceutical composition in an amount of about 0.5 wt % to about 1.25 wt %. In some embodiments, midostaurin or the pharmaceutically acceptable salt thereof is present in the topical pharmaceutical composition in an amount of about 0.8 wt % to about 1.2 wt %. In some embodiments, midostaurin or the pharmaceutically acceptable salt thereof is present in the topical pharmaceutical composition in an amount of about 1.5 wt % to about 2.5 wt %. In some embodiments, midostaurin or the pharmaceutically acceptable salt thereof is present in the topical pharmaceutical composition in an amount of about 1.75 wt % to about 2.25 wt %. In some embodiments, midostaurin or the pharmaceutically acceptable salt thereof is present in the topical pharmaceutical composition in an amount of about 1.8 wt % to about 2.2 wt %. In some embodiments, midostaurin or the pharmaceutically acceptable salt thereof is present in the topical pharmaceutical composition in an amount of about 0.1 wt %. In some embodiments, midostaurin or the pharmaceutically acceptable salt thereof is present in the topical pharmaceutical composition in an amount of about 0.2 wt %. In some embodiments, midostaurin or the pharmaceutically acceptable salt thereof is present in the topical pharmaceutical composition in an amount of about 0.25 wt %. In some embodiments, midostaurin or the pharmaceutically acceptable salt thereof is present in the topical pharmaceutical composition in an amount of about 0.3 wt %. In some embodiments, midostaurin or the pharmaceutically acceptable salt thereof is present in the topical pharmaceutical composition in an amount of about 0.4 wt %. In some embodiments, midostaurin or the pharmaceutically acceptable salt thereof is present in the topical pharmaceutical composition in an amount of about 0.5 wt %. In some embodiments, midostaurin or the pharmaceutically acceptable salt thereof is present in the topical pharmaceutical composition in an amount of about 0.75 wt %. In some embodiments, midostaurin or the pharmaceutically acceptable salt thereof is present in the topical pharmaceutical composition in an amount of about 1 wt %. In some embodiments, midostaurin or the pharmaceutically acceptable salt thereof is present in the topical pharmaceutical composition in an amount of about 1.25 wt %. In some embodiments, midostaurin or the pharmaceutically acceptable salt thereof is present in the topical pharmaceutical composition in an amount of about 1.5 wt %. In some embodiments, midostaurin or the pharmaceutically acceptable salt thereof is present in the topical pharmaceutical composition in an amount of about 2 wt %. In some embodiments, midostaurin or the pharmaceutically acceptable salt thereof is present in the topical pharmaceutical composition in an amount of about 2.5 wt %. In some embodiments, the midostaurin or the pharmaceutically acceptable salt thereof is midostaurin.

Disclosed herein are topical pharmaceutical compositions comprising midostaurin or a pharmaceutically acceptable salt thereof, a carrier vehicle, and an excipient. In some embodiments, midostaurin or the pharmaceutically acceptable salt thereof is present in the topical pharmaceutical composition in an amount of about 0.001 wt % to about 20 wt %. In some embodiments, the topical pharmaceutical composition does not comprise a phospholipid. In some embodiments, the excipient comprises a surfactant, a penetration enhancer, an antioxidant, a sun-screening agent, a viscosity modifier, a pH stabilizer, or a moisturizer, or any combination thereof. In some embodiments, the excipient comprises a surfactant. In some embodiments, the excipient comprises a penetration enhancer. In some embodiments, the excipient comprises an antioxidant. In some embodiments, the excipient comprises a sun-screening agent. In some embodiments, the excipient comprises a viscosity modifier. In some embodiments, the excipient comprises a pH stabilizer. In some embodiments, the excipient comprises a moisturizer.

In some embodiments, a topical pharmaceutical composition described herein is an ointment. In some embodiments, the topical pharmaceutical compositions comprise midostaurin or a pharmaceutically acceptable salt thereof in an amount of about 0.05% to about 5% wt. In some embodiments, the topical pharmaceutical compositions comprise a surfactant in an amount of about 5% to about 20% wt. In some embodiments, the topical pharmaceutical compositions comprise a penetration enhancer in an amount of about 0.05% to about 20% wt. In some embodiments, the topical pharmaceutical compositions comprise a penetration enhancer in an amount of about 0.05% to about 5% wt. In some embodiments, the topical pharmaceutical compositions comprise a carrier vehicle in an amount of about 70% to about 90% wt. In some embodiments, the topical pharmaceutical compositions comprise a moisturizer in an amount of about 5% to about 25% wt. In some embodiments, the topical pharmaceutical compositions comprise an antioxidant in an amount of about 0.001% to 5% wt. In some embodiments, the topical pharmaceutical composition comprises a multifunctional excipient. In some embodiments, the multifunctional excipient is simultaneously two or more of a surfactant, a penetration enhancer, a carrier vehicle, and a moisturizer.

In some embodiments, the topical pharmaceutical composition comprises midostaurin or a pharmaceutically acceptable salt thereof in an amount of 0.05% to about 2% wt. In some embodiments, the topical pharmaceutical composition comprises a surfactant, for example, glyceryl monolinoleate, glyceryl monooleate, polyoxylglycerides, glyceryl polyethylene glycol ricinoleate, polyethylene glycol-hydroxystearate or a combination thereof. In some embodiments, the topical pharmaceutical composition comprises a penetration enhancer, e.g., polyglyceryl-3 dioleate, diethylene glycol monoethyl ether, 1-dodecylazacycloheptan-2-one or Azone, or a combination thereof. In some embodiments, the topical pharmaceutical composition comprises a carrier vehicle comprising polyethylene glycol (PEG). petrolatum, mineral oil, or a combination thereof. In some embodiments, the topical pharmaceutical composition comprises a moisturizer comprising, e.g., glycerol, propylene glycol or a combination thereof. In some embodiments, the topical pharmaceutical composition comprises an antioxidant comprising, e.g., butylated hydroxytoluene (BHT), butylated hydroxyanisole (BHA), ascorbyl palmitate. a-tocopherol acetate, acetone sodium bisulfite, acetylcysteine, ascorbic acid, vitamin E, ascorbyl palmitate, cysteine, cysteine hydrochloride, or a combination thereof.

In some embodiments, the topical pharmaceutical composition comprises midostaurin or a pharmaceutically acceptable salt thereof in an amount of 0.1% to about 1.0% wt, a surfactant (e.g., caprylocaproylpolyoxy 1-8 glycerides) in an amount of about 7% to about 15% wt, a penetration enhancer in an amount of about 0.5% to about 3% wt (e.g., 1-dodecylazacycloheptan-2-one or Azone), a carrier vehicle (e.g., a polyethylene glycol pr PEG) in an amount of about 70% to about 80% wt, a moisturizer (e.g., glycerol) in an amount of about 10% to about 15% wt, and an antioxidant in an amount of about 0.001% to 1% wt. In some embodiments, the topical pharmaceutical composition comprises midostaurin or a pharmaceutically acceptable salt thereof in an amount of 0.5% to about 1.5% wt, a surfactant (e.g., caprylocaproyl polyoxyl-8 glycerides) in an amount of about 7% to about 15% wt, a penetration enhancer in an amount of about 0.5% to about 3% wt (e.g., 1-dodecylazacycloheptan-2-one or Azone), a carrier vehicle (e.g., a polyethylene glycol pr PEG) in an amount of about 70% to about 80% wt, a moisturizer (e.g., glycerol) in an amount of about 10% to about 15% wt, and an antioxidant in an amount of about 0.001% to 1% wt. In some embodiments, the antioxidant comprising butylated hydroxytoluene (BHT), butylated hydroxyanisole (BHA), ascorbyl palmitate, a-tocopherol acetate. acetone sodium bisulfite, acetylcysteine, ascorbic acid, vitamin E, ascorbyl palmitate, cysteine, cysteine hydrochloride, or a combination thereof. In some embodiments, the carrier vehicle comprises a polyethylene glycol having a number average molecular weight of about 100 to 800 g/mol or a polyethylene glycol having a number average molecular weight of about 2000 to 6000 g/mol, or a combination thereof. In some embodiments, the carrier vehicle comprises a polyethylene glycol having a number average molecular weight of about 200 to 600 g/mol or a polyethylene glycol having a number average molecular weight of about 3000 to 5000 g/mol, or a combination thereof. In some embodiments, the carrier vehicle comprises a polyethylene glycol having a number average molecular weight of about 300 to 500 g/mol or a polyethylene glycol having a number average molecular weight of about 3500 to 4500 g/mol, or a combination thereof.

In some embodiments, a topical pharmaceutical composition described herein is a gel. In some embodiments, the topical pharmaceutical compositions comprise midostaurin or a pharmaceutically acceptable salt thereof in an amount of about 0.05% to about 5% wt. In some embodiments, the topical pharmaceutical compositions comprise a surfactant in an amount of about 5% to about 50% wt. In some embodiments, the topical pharmaceutical compositions comprise optionally, a penetration enhancer in an amount of about 1% to about 30% wt. In some embodiments, the topical pharmaceutical compositions comprise a carrier vehicle in an amount of about 60% to about 95% wt. In some embodiments, the topical pharmaceutical compositions comprise a viscosity modifier in an amount of about 0.01% to about 5% wt. In some embodiments, the topical pharmaceutical compositions comprise a pH stabilizer. In some embodiments, the topical pharmaceutical compositions comprise an antioxidant in an amount of about 0.001% to 5% wt. In some embodiments, the topical pharmaceutical composition comprises a multifunctional excipient. In some embodiments, the multifunctional excipient is simultaneously a surfactant and a penetration enhancer.

In some embodiments, the topical pharmaceutical composition comprises midostaurin or a pharmaceutically acceptable salt thereof in an amount of 0.05% to about 2% wt. In some embodiments, the topical pharmaceutical composition comprises a surfactant, for example, glyceryl monolinoleate. glyceryl monooleate, polyoxylglycerides, glyceryl polyethylene glycol ricinoleate, polyoxyethylene sorbitan monooleate or a combination thereof. In some embodiments, the topical pharmaceutical composition comprises optionally a penetration enhancer, e.g., polyglyceryl-3 dioleate, oleoyl polyoxyl-6 glycerides, diethylene glycol monoethyl ether, 1-dodecylazacycloheptan-2-one or Azone, or a combination thereof. In some embodiments, the topical pharmaceutical composition comprises a viscosity modifier, for example, polyacrylic acid. In some embodiments, the topical pharmaceutical composition comprises a pH stabilizer, such as sodium hydroxide, sodium bicarbonate, L-arginine, triethanolamine, or a combination thereof. In some embodiments, the topical pharmaceutical composition comprises a carrier vehicle (e.g., water). In some embodiments, the topical pharmaceutical composition comprises an antioxidant comprising, e.g., butylated hydroxytoluene (BHT), butylated hydroxyanisole (BHA), ascorbyl palmitate, a-tocopherol acetate, acetone sodium bisulfite, acetylcysteine, ascorbic acid. vitamin E, ascorbyl palmitate, cysteine, cysteine hydrochloride, or a combination thereof.

In some embodiments, the topical pharmaceutical composition comprises midostaurin or a pharmaceutically acceptable salt thereof in an amount of 0.05% to about 1.5% wt. In some embodiments, the topical pharmaceutical composition comprises a surfactant (e.g., caprylocaproyl polyoxyl-8 glycerides or polysorbate 80) in an amount of about 5% to about 20% wt. In some embodiments, the topical pharmaceutical composition comprises optionally a penetration enhancer (e.g., diethylene glycol monoethyl ether) in an amount of about 15% to about 25% wt. In some embodiments, the topical pharmaceutical composition comprises a viscosity modifier (e.g., crosslinked polyacrylic acid such as Carbomer) in an amount of about 0.1% to about 2% wt. In some embodiments, the topical pharmaceutical composition comprises a pH stabilizer, such as triethanolamine. In some embodiments, the topical pharmaceutical composition comprises a carrier vehicle (e.g., water) in an amount of about 70% to about 90% wt. In some embodiments, the topical pharmaceutical composition comprises an antioxidant (e.g., butylated hydroxytoluene (BHT), butylated hydroxyanisole (BHA), ascorbyl palmitate. or a combination thereof) in an amount of about 0.001% to 2% wt.

In some embodiments, a topical pharmaceutical composition described herein is a cream. In some embodiments, the topical pharmaceutical compositions comprise midostaurin or a pharmaceutically acceptable salt thereof in an amount of about 0.01% to about 5% wt. In some embodiments, the topical pharmaceutical compositions comprise a carrier vehicle in an amount of about 30% to about 70% wt. In some embodiments, the topical pharmaceutical compositions comprise a surfactant in an amount of about 5% to about 50% wt. In some embodiments, the topical pharmaceutical compositions comprise optionally, a penetration enhancer in an amount of about 1% to about 40% wt. In some embodiments, the topical pharmaceutical compositions comprise a viscosity modifier in an amount of about 0.01% to about 5% wt. In some embodiments, the topical pharmaceutical compositions comprise optionally a moisturizer in an amount of about 5% to about 30% wt. In some embodiments, the topical pharmaceutical compositions comprise optionally a viscosity enhancer in an amount of about 0.01% to about 5% wt. In some embodiments, the topical pharmaceutical compositions comprise an antioxidant in an amount of about 0.001% to 5% wt.

In some embodiments, the topical pharmaceutical composition comprises midostaurin or a pharmaceutically acceptable salt thereof in an amount of 0.05% to about 5% wt. In some embodiments, the topical pharmaceutical composition comprises a carrier vehicle comprising water and optionally polyethylene glycol (PEG), propylene glycol, or liquid paraffin, or petroleum jelly (e.g., white Vaseline), or a combination thereof. In some embodiments, the topical pharmaceutical composition comprises a surfactant, for example, glyceryl monooleate, glyceryl monolinoleate, polyoxyethylene sorbitan monooleate (e.g., polyoxyethylene (20) sorbitan monooleate, Tween 80, or polysorbate 80), PEG palmitostearate (e.g., PEG-6 palmitostearate. PEG-32 palmitostearate), ethylene glycol palmitostearate, glycerol monostearate (or glyceryl monostearate, GMS), polyoxyethylene stearate (or macrogol-75 stearate), sorbitan monostearate, sodium dodecyl sulfate (SDS), polyoxyethylene alkyl ethers (e.g., Steareth-20, Steareth-2), or a combination thereof. In some embodiments, the topical pharmaceutical composition comprises optionally a moisturizer such as glycerol or propylene glycol, or a combination thereof. In some embodiments, the topical pharmaceutical composition comprises optionally a penetration enhancer, for example, oleoyl polyoxyl-6 glycerides, polyglyceryl-3 dioleate, diethylene glycol monoethyl ether, 1-dodecylazacycloheptan-2-one (or Azone), or a combination thereof. In some embodiments, the topical pharmaceutical composition comprises an antioxidant, such as butylated hydroxytoluene (BHT), butylated hydroxyanisole (BHA), ascorbyl palmitate, a-tocopherol acetate, acetone sodium bisulfite, acetylcysteine, ascorbic acid, vitamin E, ascorbyl palmitate, cysteine, cysteine hydrochloride, or a combination thereof.

In some embodiments, the topical pharmaceutical composition comprises midostaurin or a pharmaceutically acceptable salt thereof in an amount of 0.1% to about 2% wt. In some embodiments, the topical pharmaceutical composition comprises a surfactant in an amount of about 5% to about 50% wt. In some embodiments, the surfactant comprises glyceryl monooleate, glyceryl monolinoleate. polyoxyethylene sorbitan monooleate (e.g., polysorbate 80, PEG palmitostearate (e.g., PEG-6 palmitostearate, PEG-32 palmitostearate), ethylene glycol palmitostearate, glycerol monostearate. polyoxyethylene stearate (or macrogol-75 stearate), sorbitan monostearate, sodium dodecyl sulfate (SDS), or a combination thereof. In some embodiments, the topical pharmaceutical composition comprises optionally a penetration enhancer (e.g., oleoyl polyoxyl-6 glycerides, polyglyceryl-3 dioleate. diethylene glycol monoethyl ether, 1-dodecylazacycloheptan-2-one or Azone, or a combination thereof) in an amount of about 1% to about 35% wt. In some embodiments, the topical pharmaceutical composition comprises optionally a moisturizer (e.g., glycerol (glycerin) or propylene glycol, or a combination thereof) in an amount of about 5% to about 25% wt. In some embodiments, the topical pharmaceutical composition comprises an antioxidant (e.g., butylated hydroxytoluene (BHT), butylated hydroxyanisole (BHA), ascorbyl palmitate, or a combination thereof) in an amount of about 0.01% to about 2% wt. In some embodiments, the topical pharmaceutical composition comprises a carrier vehicle (e.g., water and optionally liquid paraffin, or white Vaseline) in an amount of about 30% to about 70% wt.

In some embodiments, the topical pharmaceutical composition comprises midostaurin or a pharmaceutically acceptable salt thereof in an amount of 0.5% to about 2% wt. In some embodiments, the topical pharmaceutical composition comprises a surfactant (e.g., glyceryl monolinoleate, PEG-6palmitostearate, PEG-32 palmitostearate, ethylene glycol palmitostearate, polysorbate 80, sorbitan monostearate, macrogol-75 stearate, or a combination thereof) in an amount of about 5% to about 35% wt. In some embodiments, the topical pharmaceutical composition comprises a penetration enhancer (e.g., polyglyceryl-3 dioleate, diethylene glycol monoethyl ether, 1-dodecylazacycloheptan-2-one or Azone, or a combination thereof) in an amount of about 1% to about 35% wt. In some embodiments, the topical pharmaceutical composition comprises an antioxidant (e.g., butylated hydroxytoluene (BHT), butylated hydroxyanisole (BHA), ascorbyl palmitate, or a combination thereof) in an amount of about 0.01% to about 0.1% wt. In some embodiments, the topical pharmaceutical composition comprises a carrier vehicle (e.g., water) in an amount of about 60% to about 70% wt.

In some embodiments, the topical pharmaceutical composition comprises midostaurin or a pharmaceutically acceptable salt thereof in an amount of about 1% wt. In some embodiments, the topical pharmaceutical composition comprises midostaurin or a pharmaceutically acceptable salt thereof in an amount of about 0.5% wt. In some embodiments, the topical pharmaceutical composition comprises glyceryl monooleate in an amount of about 10% wt. In some embodiments, the topical pharmaceutical composition comprises polyoxyethylene sorbitan monooleate (e.g., polysorbate 80) in an amount of about 5% to about 14% wt. In some embodiments, the topical pharmaceutical composition comprises sorbitan monostearate in an amount of about 3.5% wt. In some embodiments, the topical pharmaceutical composition comprises polyglyceryl-3 dioleate in an amount of about 5%. In some embodiments, the topical pharmaceutical composition comprises an antioxidant (e.g., butylated hydroxytoluene, butylated hydroxyanisole, and ascorbyl palmitate) in an amount of about 0.06% wt. In some embodiments, the topical pharmaceutical composition comprises water in an amount of about 66.5% wt.

In some embodiments, a topical pharmaceutical composition described herein further comprises a sun-screening agent. In some embodiments, the sun-screening agent comprises avobenzone. bemotrizinol, benzophenone-3 (BZ-3, oxy benzone), bisoctizole, homosalate, octinoxate, octisalate, octocrylene, oxybenzone, titanium dioxide, or zinc oxide, or a combination thereof.

In some embodiments, a topical pharmaceutical composition comprises midostaurin or a pharmaceutically acceptable salt thereof. In some embodiments, the topical pharmaceutical composition has a pH of about 5.0 to about 8.0, about 5.5 to about 7.5, about 6.0 to about 7.5, about 6.5 to about 7.5, about 6.8 to about 7.2, about 6.9 to about 7.1. In some embodiments, the topical pharmaceutical composition has a pH of about 5.0 to about 8.0. In some embodiments, the topical pharmaceutical composition has a pH of about 5.5 to about 7.5. In some embodiments, the topical pharmaceutical composition has a pH of about 6.0 to about 7.5. In some embodiments, the topical pharmaceutical composition has a pH of about 6.5 to about 7.5. In some embodiments, the topical pharmaceutical composition has a pH of about 6.8 to about 7.2. In some embodiments, the topical pharmaceutical composition has a pH of about 7.0.

In some embodiments, an excipient in a topical pharmaceutical composition described herein can have more than one function in the composition, i.e., a multifunctional excipient. In some embodiments, the multifunctional excipient can function as two or more of a surfactant, a penetration enhancer, a viscosity modifier, a carrier vehicle, and a moisturizer. In some embodiments, the multifunctional excipient can function as a surfactant and a penetration enhancer, such as oleoyl polyoxyl-6 glycerides (e.g., sold under the trademark Labrafil® M 1944 CS), polyglyceryl-3 dioleate (e.g., e.g., sold under the trademark Plurol R® Oleique CC 497). In some embodiments, the multifunctional excipient can function as a moisturizer and a carrier vehicle, such as propylene glycol or glycerol.

### Carrier Vehicle

In one aspect, disclosed herein are topical pharmaceutical compositions comprising midostaurin or a pharmaceutically acceptable salt thereof and a carrier vehicle. The carrier vehicle can be any suitable carrier vehicle that can deliver midostaurin or a pharmaceutically acceptable salt thereof in a topical pharmaceutical composition. The carrier vehicle can be any suitable carrier vehicle that can deliver midostaurin or a pharmaceutically acceptable salt thereof and a suitable excipient. The carrier vehicle can be any suitable carrier vehicle that can dissolve midostaurin or a pharmaceutically acceptable salt thereof and can form a mixture that can maintain acceptable physical and chemical stability. In some embodiments, the midostaurin or a pharmaceutically acceptable salt thereof is dissolved in the carrier vehicle. In some embodiments, the carrier vehicle comprises an aqueous carrier vehicle. In some embodiments, the carrier vehicle comprises water. In some embodiments, the carrier vehicle comprises a non-aqueous carrier vehicle. In some embodiments, the carrier vehicle does not comprise water. The non-aqueous carrier can include oils (e.g., edible vegetable oils or synthetic edible oils), propylene glycol, glycerin, polypropylene glycol, polyethylene glycol (PEG), alcohol (e.g., ethanol), or any combinations thereof. In some embodiments, the carrier vehicle comprises polyethylene glycol (PEG), propylene glycol, glycerin (or glycerol), dimethyl sulfoxide (DMSO), petrolatum, mineral oil, wax, liquid paraffin, petroleum jelly, or a combination thereof. In some embodiments, the carrier vehicle comprises water, polyethylene glycol, propylene glycol, or glycerin, or a combination thereof. In some embodiments, the carrier vehicle comprises water. In some embodiments, the carrier vehicle comprises water, liquid paraffin, petroleum jelly, or a combination thereof.

In some embodiments, the carrier vehicle comprises propylene glycol. In some embodiments, the carrier vehicle comprises glycerin. In some embodiments, the carrier vehicle comprises PEG. In some embodiments, the PEG has an average molecular weight of about 100 to about 10,000 g/mol. In some embodiments, the PEG has an average molecular weight of about 100 to about 500 g/mol, about 500 to about 1000 g/mol, about 1000 to about 5000 g/mol, about 5000 to about 10,000 g/mol. In some embodiments, the PEG has an average molecular weight of about 200 to about 500 g/mol. In some embodiments, the PEG has an average molecular weight of about 300 to about 500 g/mol. In some embodiments, the PEG has an average molecular weight of about 350 to about 450 g/mol. In some embodiments, the PEG has an average molecular weight of about 400 g/mol. In some embodiments, the PEG is PEG 400.

In some embodiments, the carrier vehicle comprises PEG. In some embodiments, the PEG has a number average molecular weight of about 200 to about 20.000 g/mol. In some embodiments, the PEG has a number average molecular weight of about 200 to about 500 g/mol, about 500 to about 1000 g/mol, about 1000 to about 8000 g/mol, about 8000 to about 12,000 g/mol, about 12,000 to about 20,000 g/mol. In some embodiments, the PEG has a number average molecular weight of about 1000 to about 8000 g/mol, about 2000 to about 7000 g/mol, about 3000 to about 6000 g/mol, about 3500 to about 5500 g/mol, about 3500 to about 5000 g/mol, about 3700 to about 4500 g/mol, about 3800 to about 4200 g/mol. In some embodiments, the PEG has a number average molecular weight of about 1000 to about 8000 g/mol. In some embodiments, the PEG has a number average molecular weight of about 2000 to about 7000 g/mol. In some embodiments, the PEG has a number average molecular weight of about 3000 to about 6000 g/mol. In some embodiments, the PEG has a number average molecular weight of about 3500 to about 5500 g/mol. In some embodiments, the PEG has a number average molecular weight of about 3500 to about 5000 g/mol. In some embodiments, the PEG has a number average molecular weight of about 3700 to about 4500 g/mol. In some embodiments, the PEG has a number average molecular weight of about 3800 to about 4200 g/mol. In some embodiments, the PEG has a number average molecular weight of about 4000 g/mol. In some embodiments, the PEG is PEG 4000.

In some embodiments, a carrier vehicle is present in the topical pharmaceutical composition described herein in an amount of about 35 wt % to about 99 wt %. In some embodiments, the carrier vehicle is present in the topical pharmaceutical composition in an amount of about 35 wt % to about 40 wt %, about 40) wt % to about 45 wt %, about 45 wt % to about 50 wt %, about 50 wt % to about 55 wt %, about 55 wt % to about 60) wt %, about 60 wt % to about 65 wt %, about 65 wt % to about 70 wt %, about 70) wt % to about 75 wt %, about 75 wt % to about 80 wt %, about 80 wt % to about 85 wt %, about 85 wt % to about 90 wt %, about 90 wt % to about 93 wt %, about 93 wt % to about 96 wt %, or about 96 wt % to about 99 wt %. In some embodiments, the carrier vehicle is present in the topical pharmaceutical composition in an amount of about 35 wt % to about 40 wt %. In some embodiments, the carrier vehicle is present in the topical pharmaceutical composition in an amount of about 40 wt % to about 45 wt %. In some embodiments, the carrier vehicle is present in the topical pharmaceutical composition in an amount of about 45 wt % to about 50 wt %. In some embodiments, the carrier vehicle is present in the topical pharmaceutical composition in an amount of about 50 wt % to about 55 wt %. In some embodiments, the carrier vehicle is present in the topical pharmaceutical composition in an amount of about 55 wt % to about 60 wt %. In some embodiments, the carrier vehicle is present in the topical pharmaceutical composition in an amount of about 60 wt % to about 65 wt %. In some embodiments, the carrier vehicle is present in the topical pharmaceutical composition in an amount of about 65 wt % to about 70 wt %. In some embodiments, the carrier vehicle is present in the topical pharmaceutical composition in an amount of about 70 wt % to about 75 wt %. In some embodiments, the carrier vehicle is present in the topical pharmaceutical composition in an amount of about 75 wt % to about 80 wt %. In some embodiments, the carrier vehicle is present in the topical pharmaceutical composition in an amount of about 80 wt % to about 85 wt %. In some embodiments, the carrier vehicle is present in the topical pharmaceutical composition in an amount of about 85 wt % to about 90 wt %. In some embodiments, the carrier vehicle is present in the topical pharmaceutical composition in an amount of about 70 wt % to about 95 wt %. In some embodiments, the carrier vehicle is present in the topical pharmaceutical composition in an amount of about 90 wt % to about 99 wt %. In some embodiments, the carrier vehicle is present in the topical pharmaceutical composition in an amount of about 90 wt % to about 93 wt %. In some embodiments, the carrier vehicle is present in the topical pharmaceutical composition in an amount of about 93 wt % to about 96 wt %. In some embodiments, the carrier vehicle is present in the topical pharmaceutical composition in an amount of about 96 wt % to about 99 wt %. In some embodiments, the carrier vehicle comprises water. In some embodiments, the carrier vehicle comprises PEG 400. In some embodiments, the carrier vehicle comprises PEG 4000. In some embodiments, the carrier vehicle comprises PEG 400 and PEG 4000. In some embodiments, the carrier vehicle comprises glycerin. In some embodiments, the carrier vehicle comprises PEG 400. PEG 4000, and glycerin. In some embodiments, the carrier vehicle comprises propylene glycol. In some embodiments, the carrier vehicle comprises water and propylene glycol.

In some embodiments, the carrier vehicle comprises water. In some embodiments, water is present in the topical pharmaceutical composition described herein in an amount of about 35 wt % to about 95 wt %. In some embodiments, water is present in the topical pharmaceutical composition in an amount of about 35 wt % to about 40 wt %, about 40 wt % to about 45 wt %, about 45 wt % to about 50 wt %, about 50 wt % to about 55 wt %, about 55 wt % to about 60 wt %, about 60 wt % to about 65 wt %, about 65 wt % to about 70 wt %, about 70 wt % to about 75 wt %, about 75 wt % to about 80 wt %, about 80 wt % to about 85 wt %, about 85 wt % to about 90 wt %, or about 90 wt % to about 95 wt %. In some embodiments, water is present in the topical pharmaceutical composition in an amount of about 35 wt % to about 40 wt %. In some embodiments, water is present in the topical pharmaceutical composition in an amount of about 40 wt % to about 45 wt %. In some embodiments, water is present in the topical pharmaceutical composition in an amount of about 45 wt % to about 50 wt %. In some embodiments, water is present in the topical pharmaceutical composition in an amount of about 50 wt % to about 55 wt %. In some embodiments, water is present in the topical pharmaceutical composition in an amount of about 55 wt % to about 60 wt %. In some embodiments, water is present in the topical pharmaceutical composition in an amount of about 60 wt % to about 65 wt %. In some embodiments, water is present in the topical pharmaceutical composition in an amount of about 65 wt % to about 70 wt %. In some embodiments, water is present in the topical pharmaceutical composition in an amount of about 70 wt % to about 75 wt %. In some embodiments, water is present in the topical pharmaceutical composition in an amount of about 75 wt % to about 80 wt %. In some embodiments, water is present in the topical pharmaceutical composition in an amount of about 80 wt % to about 85 wt %. In some embodiments,

water is present in the topical pharmaceutical composition in an amount of about 85 wt % to about 90 wt %. In some embodiments, water is present in the topical pharmaceutical composition in an amount of about 90 wt % to about 95 wt %. In some embodiments, water is present in the topical pharmaceutical composition in an amount of about 40 wt %. In some embodiments, water is present in the topical pharmaceutical composition in an amount of about 50 wt %. In some embodiments, water is present in the topical pharmaceutical composition in an amount of about 60 wt %. In some embodiments, water is present in the topical pharmaceutical composition in an amount of about 65 wt %. In some embodiments, water is present in the topical pharmaceutical composition in an amount of about 67.5 wt %.

In some embodiments, the carrier vehicle comprises water, propylene glycol, and glycerin. In some embodiments, the carrier vehicle comprises propylene glycol and glycerin in a weight ratio of from about 1:1 to about 5:1, about 2:1 to about 4:1, about 2.5:1 to about 3.5:1. In some embodiments, the carrier vehicle comprises propylene glycol and glycerin in a weight ratio of about 2.5:1 to about 3.5:1. In some embodiments, the carrier vehicle comprises propylene glycol, and glycerin in a weight ratio of about 3:1. In some embodiments, the carrier vehicle comprises water and propylene glycol in a weight ratio of from about 1:1 to about 5:1, about 2:1 to about 4:1, about 2.5:1 to about 3.5:1. In some embodiments, the carrier vehicle comprises water and propylene glycol in a weight ratio of about 2.5:1 to about 3.5:1. In some embodiments, the carrier vehicle comprises water and propylene glycol in a weight ratio of about 3:1 to about 10:3.

In some embodiments, the carrier vehicle comprises water, liquid paraffin, and petroleum jelly (e.g., white Vaseline). In some embodiments, the carrier vehicle comprises liquid paraffin and petroleum jelly (e.g., white Vaseline) in a weight ratio of from about 1:5 to about 5:1, about 1:4 to about 4:1, about 1:3 to about 3:1. In some embodiments, the carrier vehicle comprises liquid paraffin and petroleum jelly (e.g., white Vaseline) in a weight ratio of about 1:1 to about 3:1. In some embodiments, the carrier vehicle comprises liquid paraffin and petroleum jelly (e.g., white Vaseline) in a weight ratio of about 5:3. In some embodiments, the carrier vehicle comprises water and liquid paraffin in a weight ratio of from about 20:1 to about 5:1, about 15:1 to about 6:1, about 12:1 to about 8:1. In some embodiments, the carrier vehicle comprises water and liquid paraffin in a weight ratio of about 12:1 to about 8:1. In some embodiments, the carrier vehicle comprises water liquid paraffin in a weight ratio of about 11:1 to about 9:1. In some embodiments, the carrier vehicle comprises water liquid paraffin in a weight ratio of about 10:1.

In some embodiments, the carrier vehicle comprises PEG. In some embodiments, the carrier vehicle does not comprise water. In some embodiments, the carrier vehicle comprises PEG 400. In some embodiments, the carrier vehicle comprises PEG 4000. In some embodiments, the carrier vehicle comprises PEG 400 and PEG 4000. In some embodiments, the carrier vehicle comprises PEG 400 and PEG 4000 in a weight ratio of from about 1:1 to about 10:1, about 2:1 to about 9:1, about 3:1 to about 8:1, about 4:1 to about 7:1, about 5:1 to about 7:1. In some embodiments, the carrier vehicle comprises PEG 400 and PEG 4000 in a weight ratio of about 5:1 to about 7:1. In some embodiments, the carrier vehicle comprises PEG 400 and PEG 4000 in a weight ratio of about 5.5:1 to about 6.5:1. In some embodiments, the carrier vehicle comprises PEG 400 and PEG 4000 in a weight ratio of about 6.08:1. In some embodiments. PEG is present in the topical pharmaceutical composition in an amount of about 60 wt % to about 85 wt %. In some embodiments, PEG is present in the topical pharmaceutical composition in an amount of about 60 wt % to about 65 wt %, about 65 wt % to about 70 wt %, about 70 wt % to about 75 wt %, about 75 wt % to about 80 wt %, or about 80 wt % to about 85 wt %. In some embodiments. PEG is present in the topical pharmaceutical composition in an amount of about 35 wt % to about 40 wt %. In some embodiments, PEG is present in the topical pharmaceutical composition in an amount of about 40 wt % to about 45 wt %. In some embodiments, PEG is present in the topical pharmaceutical composition in an amount of about 45 wt % to about 50 wt %. In some embodiments, PEG is present in the topical pharmaceutical composition in an amount of about 50 wt % to about 55 wt %. In some embodiments, PEG is present in the topical pharmaceutical composition in an amount of about 55 wt % to about 60 wt %. In some embodiments, PEG is present in the topical pharmaceutical composition in an amount of about 60 wt % to about 65 wt %. In some embodiments, PEG is present in the topical pharmaceutical composition in an amount of about 65 wt % to about 70 wt %. In some embodiments. PEG is present in the topical pharmaceutical composition in an amount of about 70 wt % to about 75 wt %. In some embodiments, PEG is present in the topical pharmaceutical composition in an amount of about 75 wt % to about 80 wt %. In some embodiments, PEG is present in the topical pharmaceutical composition in an amount of about 70 wt % to about 75 wt %. In some embodiments, PEG is present in the topical pharmaceutical composition in an amount of about 75 wt %.

### Surfactant

In one aspect, disclosed herein are topical pharmaceutical compositions comprising midostaurin or a pharmaceutically acceptable salt thereof, a carrier vehicle, and an excipient (e.g., comprising a surfactant).

In some embodiments, the topical pharmaceutical compositions described herein comprise a surfactant. In some embodiments, surfactants are compounds or mixture of compounds comprising a hydrophobic group (usually a hydrocarbon chain) and a hydrophilic group. In some embodiments, surfactants can include emulsifiers. Surfactants can perform one or more roles including solubility enhancer, bioavailability enhancer, penetration enhancer, and emulsifying agent. Examples of surfactants include, but are not limited to kolliphor series (rh40), sorbitan oleate, SDS, solutal, soluplus, sucrose esters of fatty acids, polyoxyl stearate, polyoxyethylene hydrogenated castor oil, polyoxyl 40 hydrogenated castor, macrogolglycerol hydroxystearate oil, peg-40 castor oil, polyoxyethylene polyoxypropylene glycol, sorbitan sesquioleate, sorbitan trioleate, sorbitan monostearate, sorbitan monopalmitate, sorbitan monolaurate, polysorbate, glyceryl monostearate, sodium lauryl sulfate, sodium dodecyl sulfate, lauromacrogol arlasolve, poloxamers, labrafil, labrasol, tween 80, tocopheryl polyethylene glycol 1000 succinate (simply TPGS or vitamin E TPGS) and the like.

The surfactant used in the present disclosure can comprise one or more non-ionic surfactants, one or more ionic surfactants, one or more zwitterionic surfactants, or a mixture thereof. In some embodiments, a non-ionic surfactant has no charged groups in its head. Exemplary nonionic surfactants include, without limitation, fatty alcohols, cetyl alcohol, stearyl alcohol, cetostearyl alcohol, and oleyl alcohol. Exemplary nonionic surfactants include, but are not limited to, polyethylene glycol alkyl ethers (such as octaethylene glycol monododecyl ether, pentaethylene glycol monododecyl ether). polypropylene glycol alkyl ethers, glucoside alkyl ethers (such as decyl glucoside, lauryl glucoside, octyl glucoside), polyethylene glycol octylphenyl ethers (such as Triton X-100), polyethylene glycol alkylphenyl ethers (such as nonoxynol-9), glycerol alkyl esters (such as glyceryl laurate). polyoxyethylene glycol sorbitan alkyl esters (such as polysorbate), sorbitan alkyl esters (such as Spans), cocamide MEA, cocamide DEA, block copolymers of polyethylene glycol and polypropylene glycol (such as poloxamers), polyethoxylated tallow amine (POEA), and Tocopheryl polyethylene glycol 1000 succinate (simply TPGS or Vitamin E TPGS). In some embodiments, a non-ionic surfactant comprises one or more of fatty alcohols, e.g., cetyl alcohol, stearyl alcohol, cetostearyl alcohol, and oleyl alcohol. In some embodiments, the non-ionic surfactant comprises a C₉-C₃₀ fatty alcohol or fatty acid. In some embodiments, the non-ionic surfactant comprises a C₁₂-C₂₄ fatty alcohol or fatty acid. In some embodiments, the non-ionic surfactant comprises a C₁₂-C₁₈ fatty alcohol or fatty acid. Exemplary nonionic surfactants include, but are not limited to, polyethylene glycol alkyl ethers (such as octaethylene glycol monododecyl ether, pentaethylene glycol monododecyl ether), polypropylene glycol alkyl ethers. glucoside alkyl ethers (such as decyl glucoside, lauryl glucoside, octyl glucoside), polyethylene glycol octylphenyl ethers (such as Triton X-100), polyethylene glycol alkylphenyl ethers (such as nonoxynol-9). glycerol alkyl esters (such as glyceryl laurate), polyoxyethylene glycol sorbitan alkyl esters (such as polysorbate), sorbitan alkyl esters (such as Spans), cocamide MEA, cocamide DEA. dodecyldimethylamine oxide, block copolymers of polyethylene glycol and polypropylene glycol (such as poloxamers), polyethoxylated tallow amine (POEA), and Tocopheryl polyethylene glycol 1000 succinate (simply TPGS or Vitamin E TPGS).

In some embodiments, the topical pharmaceutical composition comprises a surfactant. In some embodiments, the topical pharmaceutical composition comprises two or more surfactants. In some embodiments, the topical pharmaceutical composition comprises two, three, four or more surfactants. In some embodiments, the topical pharmaceutical composition comprises two surfactants. In some embodiments, the topical pharmaceutical composition comprises three surfactants. In some embodiments, the topical pharmaceutical composition comprises four surfactants.

In some embodiments, the surfactant comprises glyceryl monooleate (e.g., sold under the trademark Peceol™). In some embodiments, the surfactant comprises glyceryl monolinoleate (e.g., sold under the trademark Maisine® CC). In some embodiments, the surfactant comprises polyethylene glycol-hydroxystearate, such as macrogol-15-hydroxystearate (e.g., sold under the trademark Kolliphor® HS15). In some embodiments, the surfactant comprises polyoxyethylene sorbitan monooleate, for example, polyoxyethylene (20) sorbitan monooleate. Tween 80, or polysorbate 80. In some embodiments, the surfactant comprises polyoxylglycerides, such as caprylocaproyl polyoxyl-8 glycerides (e.g., sold under the trademark Labarasol®). In some embodiments, the surfactant comprises oleoyl polyoxyl-6 glycerides (e.g., sold under the trademark Labrafil® M 1944 CS). In some embodiments, the surfactant comprises glyceryl polyethylene glycol ricinoleate, for example, polyoxyl 35 hydrogenated castor oil (e.g., sold under the trademark Cremophor® EL). In some embodiments, the surfactant comprises PEG palmitostearate (e.g., PEG-6 palmitostearate, PEG-32 palmitostearate). In some embodiments, the surfactant comprises ethylene glycol palmitostearate (e.g., ethylene glycol monopalmitostearate). In some embodiments, the surfactant comprises a mixture of PEG-6 palmitostearate. PEG-32 palmitostearate, and ethylene glycol monopalmitostearate (e.g., sold under the trademark Tefose® 63). In some embodiments, the surfactant comprises glycerol monostearate (or glyceryl monostearate, GMS). In some embodiments, the surfactant comprises polyoxyethylene stearate and macrogol stearate (e.g., mixture of glycerol monostearate and macrogol-75 stearate, sold under the trademark Gelot™ 64). In some embodiments, the surfactant comprises sorbitan monostearate (e.g., sold under the trademark Span® 60. In some embodiments, the surfactant comprises polyglyceryl-3 dioleate (e.g., sold under the tradename Plurol® Oleique CC 497). In some embodiments, the surfactant comprises Sodium dodecyl sulfate (SDS). In some embodiments, the surfactant comprises polyoxyethylene alkyl ethers (e.g., Steareth-20, Steareth-2).

In some embodiments, the surfactant comprises an emulsifier. In some embodiments, the emulsifier comprises polyoxyethylene sorbitan monooleate (e.g., polyoxyethylene (20) sorbitan monooleate, Tween 80, or polysorbate 80, PEG palmitostearate (e.g., PEG-6 palmitostearate, PEG-32 palmitostearate), ethylene glycol palmitostearate, glycerol monostearate (or glyceryl monostearate. GMS), polyoxyethylene stearate (or macrogol stearate), sorbitan monostearate, sodium dodecyl sulfate (SDS), oleoyl polyoxyl-6 glycerides, or polyoxyethylene alkyl ethers (e.g., Steareth-20, Steareth-2), or a combination thereof.

In some embodiments, a surfactant comprises fatty acids, sphingolipids, saccharolipids. polyketides, sterol lipids, prenol lipids and the like. In some embodiments, a surfactant does not comprise a phospholipid.

In some embodiments, the surfactant comprises a non-ionic surfactant. In some embodiments, the non-ionic surfactant comprises Vitamin E, a block copolymer of polyethylene glycol and polypropylene glycol, or any combination thereof. In some embodiments, the surfactant comprises two more repeating units, such as polyoxyalkylene units. In some embodiments, the surfactant is a non-ionic surfactant that comprises polyethylene glycol. In some embodiments, the surfactant is a block copolymer of polyethylene glycol and polypropylene glycol.

In some embodiments, the surfactant comprises an ionic surfactant. An ionic surfactant can have a charged group in its head. An ionic surfactant can have an anionic head group or a cationic head group. In some embodiments, exemplary ionic surfactants include sodium lauryl sulfate (SLS), sodium dodecyl sulfate, calcium oleate, triethanolamine oleate, docusate sodium, benzalkonium chloride, and cetylpyridinium chloride. In some embodiments, the pharmaceutical composition or the topical pharmaceutical composition comprises SLS. In some embodiments, the surfactant is a mixture of one or more non-ionic surfactants and one or more ionic surfactant. In some embodiments, the surfactant comprises SLS and TPGS.

In some embodiments, the non-ionic surfactant has a number average molecular weight of from about from about 1000 to about 100,000 Da, 2000 to about 20,000 Da, from about 4000 to about 15,000 Da, from about 6000 to about 12,000 Da, or from about 7000 to about 10,000 Da. In some embodiments, the non-ionic surfactant has a number average molecular weight of from about 7000 to about 10,000 Da. In some embodiments, the non-ionic surfactant has an ethylene glycol content of from about 30 wt % to about 99 wt %, from about 50 wt % to about 95 wt %, from about 60 wt % to about 95 wt %, from about 75 wt % to about 90 wt %, or from about 80 wt % to about 85 wt %. In some embodiments, the non-ionic surfactant has an ethylene glycol content of from about 80 wt % to about 85 wt %.

In some embodiments, a surfactant is present in the topical pharmaceutical composition in an amount of about 0.01 wt % to about 55 wt %. In some embodiments, the surfactant is present in the topical pharmaceutical composition in an amount of about 0.01 wt % to about 1 wt %, about 1 wt % to about 5 wt %, about 5 wt % to about 10 wt %, about 10 wt % to about 15 wt %, about 15 wt % to about 20 wt %, about 20 wt % to about 25 wt %, about 25 wt % to about 30 wt %, about 30 wt % to about 40 wt %, about 40 wt % to about 45 wt %, about 45 wt % to about 50 wt %, or about 50 wt % to about 55 wt %. In some embodiments, the surfactant is present in the topical pharmaceutical composition in an amount of about 0.01 wt % to about 1 wt %. In some embodiments, the surfactant is present in the topical pharmaceutical composition in an amount of about 1 wt % to about 5 wt %. In some embodiments, the surfactant is present in the topical pharmaceutical composition in an amount of about 5 wt % to about 10 wt %. In some embodiments, the surfactant is present in the topical pharmaceutical composition in an amount of about 10 wt % to about 15 wt %. In some embodiments, the surfactant is present in the topical pharmaceutical composition in an amount of about 20 wt % to about 25 wt %. In some embodiments, the surfactant is present in the topical pharmaceutical composition in an amount of about 25 wt % to about 30 wt %. In some embodiments, the surfactant is present in the topical pharmaceutical composition in an amount of about 30 wt % to about 35 wt %. In some embodiments, the surfactant is present in the topical pharmaceutical composition in an amount of about 35 wt % to about 40 wt %. In some embodiments, the surfactant is present in the topical pharmaceutical composition in an amount of about 40 wt % to about 45 wt %. In some embodiments, the surfactant is present in the topical pharmaceutical composition in an amount of about 45 wt % to about 50 wt %. In some embodiments, the surfactant is present in the topical pharmaceutical composition in an amount of about 50 wt % to about 55 wt %. In some embodiments, the surfactant comprises polyoxylglycerides (e.g., caprylocaproyl polyoxyl-8 glycerides). In some embodiments, the surfactant comprises polyoxyethylene sorbitan monooleate (e.g., polyoxyethylene (20) sorbitan monooleate, Tween 80, or polysorbate 80. In some embodiments, the surfactant comprises oleoyl polyoxyl-6 glycerides. In some embodiments, the surfactant comprises PEG-6 palmitostearate. PEG-32 palmitostearate, and ethylene glycol palmitostearate (e.g., Tefose® 63). In some embodiments, the surfactant comprises polyoxyethylene stearate or macrogol stearate (e.g., Gelot™ 64). In some embodiments, the surfactant comprises sorbitan monostearate (e.g., Span® 60. In some embodiments, the surfactant comprises polyglyceryl-3 dioleate (e.g., Plurol® Oleique CC 497) . In some embodiments, the surfactant comprises sodium dodecyl sulfate (SDS). In some embodiments, the surfactant comprises glyceryl monooleate (e.g., Peceol™).

In some embodiments, glyceryl monooleate (e.g., Peceol™) is present in the topical pharmaceutical composition in an amount of about 1 wt % to about 20 wt %. In some embodiments, glyceryl monooleate (e.g., Peceol™) is present in the topical pharmaceutical composition in an amount of about 1 wt % to about 5 wt %, about 5 wt % to about 10 wt %, about 10 wt % to about 15 wt %, or about 15 wt % to about 20 wt %. In some embodiments, glyceryl monooleate (e.g., Peceol™) is present in the topical pharmaceutical composition in an amount of about 1 wt % to about 5 wt %. In some embodiments, glyceryl monooleate (e.g., Peceol™) is present in the topical pharmaceutical composition in an amount of about 5 wt % to about 10 wt %. In some embodiments, glyceryl monooleate (e.g., Peceol™) is present in the topical pharmaceutical composition in an amount of about 10 wt % to about 15 wt %. In some embodiments, glyceryl monooleate (e.g., Peceol™) is present in the topical pharmaceutical composition in an amount of about 15 wt % to about 20 wt %. In some embodiments, glyceryl monooleate (e.g., Peceol™) is present in the topical pharmaceutical composition in an amount of about 10 wt %.

In some embodiments, polyoxyethylene sorbitan monooleate (e.g., polyoxyethylene (20) sorbitan monooleate. Tween 80, or polysorbate 80) is present in the topical pharmaceutical composition in an amount of about 1 wt % to about 25 wt %. In some embodiments, polyoxyethylene sorbitan monooleate (e.g., polyoxyethylene (20) sorbitan monooleate. Tween 80, or polysorbate 80) is present in the topical pharmaceutical composition in an amount of about 1 wt % to about 5 wt %, about 5 wt % to about 10 wt %, about 10 wt % to about 15 wt %, about 15 wt % to about 20 wt %, or about 20 wt % to about 25 wt %. In some embodiments, polyoxyethylene sorbitan monooleate (e.g., polyoxyethylene (20) sorbitan monooleate. Tween 80, or polysorbate 80) is present in the topical pharmaceutical composition in an amount of about 1 wt % to about 5 wt %. In some embodiments, polyoxyethylene sorbitan monooleate (e.g., polyoxyethylene (20) sorbitan monooleate. Tween 80, or polysorbate 80) is present in the topical pharmaceutical composition in an amount of about 5 wt % to about 10 wt %. In some embodiments, polyoxyethylene sorbitan monooleate (e.g., polyoxyethylene (20) sorbitan monooleate. Tween 80, or polysorbate 80) is present in the topical pharmaceutical composition in an amount of about 10 wt % to about 15 wt %. In some embodiments, polyoxyethylene sorbitan monooleate (e.g., polyoxyethylene (20) sorbitan monooleate. Tween 80, or polysorbate 80) is present in the topical pharmaceutical composition in an amount of about 15 wt % to about 20 wt %. In some embodiments, polyoxyethylene sorbitan monooleate (e.g., polyoxyethylene (20) sorbitan monooleate. Tween 80, or polysorbate 80) is present in the topical pharmaceutical composition in an amount of about 20 wt % to about 25 wt %. In some embodiments, polyoxyethylene sorbitan monooleate (e.g., polyoxyethylene (20) sorbitan monooleate. Tween 80, or polysorbate 80) is present in the topical pharmaceutical composition in an amount of about 14 wt %.

In some embodiments, sorbitan monostearate (e.g., Span R 60) is present in the topical pharmaceutical composition in an amount of about 1 wt % to about 10 wt %. In some embodiments, sorbitan monostearate (e.g., Span R 60) is present in the topical pharmaceutical composition in an amount of about 1 wt % to about 2 wt %, about 2 wt % to about 5 wt %, about 5 wt % to about 7 wt %, or about 7 wt % to about 10 wt %. In some embodiments, sorbitan monostearate (e.g., Span® 60) is present in the topical pharmaceutical composition in an amount of about 1 wt % to about 2 wt %. In some embodiments, sorbitan monostearate (e.g., Span® 60) is present in the topical pharmaceutical composition in an amount of about 2 wt % to about 5 wt %. In some embodiments, sorbitan monostearate (e.g., Span® 60) is present in the topical pharmaceutical composition in an amount of about 5 wt % to about 7 wt %. In some embodiments, sorbitan monostearate (e.g., Span® 60) is present in the topical pharmaceutical composition in an amount of about 7 wt % to about 10 wt %. In some embodiments, sorbitan monostearate (e.g., Span® 60) is present in the topical pharmaceutical composition in an amount of about 3.5 wt %.

In some embodiments, polyglyceryl-3 dioleate (e.g., Plurol® Oleique CC 497) is present in the topical pharmaceutical composition in an amount of about 1 wt % to about 10 wt %. In some embodiments, polyglyceryl-3 dioleate (e.g., Plurol® Oleique CC 497) is present in the topical pharmaceutical composition in an amount of about 1 wt % to about 2 wt %, about 2 wt % to about 5 wt %, about 5 wt % to about 7 wt %, or about 7 wt % to about 10 wt %. In some embodiments, polyglyceryl-3 dioleate (e.g., Plurol® Oleique CC 497) is present in the topical pharmaceutical composition in an amount of about 1 wt % to about 2 wt %. In some embodiments, polyglyceryl-3 dioleate (e.g., Plurol® Oleique CC 497) is present in the topical pharmaceutical composition in an amount of about 2 wt % to about 5 wt %. In some embodiments, polyglyceryl-3 dioleate (e.g., Plurol® Oleique CC 497) is present in the topical pharmaceutical composition in an amount of about 5 wt % to about 7 wt %. In some embodiments, polyglyceryl-3 dioleate (e.g., Plurol® Oleique CC 497) is present in the topical pharmaceutical composition in an amount of about 7 wt % to about 10 wt %. In some embodiments, polyglyceryl-3 dioleate (e.g., Plurol® Oleique CC 497) is present in the topical pharmaceutical composition in an amount of about 5 wt %.

### Penetration Enhancer

In one aspect, disclosed herein are topical pharmaceutical compositions comprising midostaurin or a pharmaceutically acceptable salt thereof, a carrier vehicle, and an excipient (e.g., comprising a penetration enhancer).

In some embodiments, the topical pharmaceutical compositions described herein comprise a penetration enhancer. Penetration enhancer can penetrate into skin, interact with skin constituents. decrease the skin barrier resistance, and promote flux of a drug, such as midostaurin or a pharmaceutically acceptable salt thereof in topical pharmaceutical compositions described herein. In some embodiments, penetration enhancers can simultaneously possess one or more roles including surfactant, solubility enhancer, bioavailability enhancer, and emulsifying agent. Other terms in the art for penetration enhancer include penetration-enhancing agent, penetration agent, sorption promoters. accelerants and the like. Examples of penetration enhancers include, but are not limited to pyrrolidones (e.g., 2-pyrrolidone), alcohols (e.g., ethanol), esters, alkanols (e.g., decanol), esters sulfoxides (such as dimethyl sulfoxide) and their derivatives, glycols (e.g., propylene glycol), hydrocarbons, terpenes and derivatives. Azone and its analogs, amides (including urea and its derivatives), fatty acids, surfactants (nonionic, cationic, and anionic), oleodendrimers, ionic liquids, and deep eutectic solvents. In some embodiments, the topical pharmaceutical compositions described herein comprise two or more penetration enhancers.

In some embodiments, the penetration enhancer comprises polyglyceryl-3 dioleate (e.g., Plurol® Oleique CC 497), diethylene glycol monoethyl ether (e.g., sold under the trademark Transcutol® P). oleoyl polyoxyl-6 glycerides (e.g., Labrafil® M 1944 CS), or 1-dodecylazacycloheptan-2-one (or Azone), or a combination thereof.

In some embodiments, a penetration enhancer is present in the topical pharmaceutical composition in an amount of about 0.01 wt % to about 45 wt %. In some embodiments, the penetration enhancer is present in the topical pharmaceutical composition in an amount of about 0.01 wt % to about 1 wt %, about 1 wt % to about 5 wt %, about 5 wt % to about 10 wt %, about 10 wt % to about 15 wt %. about 15 wt % to about 20 wt %, about 20 wt % to about 25 wt %, about 25 wt % to about 30 wt %. about 30 wt % to about 40 wt %, or about 40 wt % to about 45 wt %. In some embodiments, the penetration enhancer is present in the topical pharmaceutical composition in an amount of about 0.01 wt % to about 1 wt %. In some embodiments, the penetration enhancer is present in the topical pharmaceutical composition in an amount of about 1 wt % to about 5 wt %. In some embodiments, the penetration enhancer is present in the topical pharmaceutical composition in an amount of about 5 wt % to about 10 wt %. In some embodiments, the penetration enhancer is present in the topical pharmaceutical composition in an amount of about 10 wt % to about 15 wt %. In some embodiments, the penetration enhancer is present in the topical pharmaceutical composition in an amount of about 20 wt % to about 25 wt %. In some embodiments, the penetration enhancer is present in the topical pharmaceutical composition in an amount of about 25 wt % to about 30 wt %. In some embodiments, the penetration enhancer is present in the topical pharmaceutical composition in an amount of about 30 wt % to about 35 wt %. In some embodiments, the penetration enhancer is present in the topical pharmaceutical composition in an amount of about 35 wt % to about 40 wt %. In some embodiments, the penetration enhancer is present in the topical pharmaceutical composition in an amount of about 40 wt % to about 45 wt %. In some embodiments, the penetration enhancer is present in the topical pharmaceutical composition in an amount of about 45 wt % to about 50 wt %. In some embodiments, the penetration enhancer comprises polyglyceryl-3 dioleate (e.g., Plurol® Oleique CC 497). In some embodiments, the penetration enhancer comprises diethylene glycol monoethyl ether (e.g., Transcutol® P). In some embodiments, the penetration enhancer comprises oleoyl polyoxyl-6 glycerides (e.g., Labrafil® M 1944 CS). In some embodiments, the penetration enhancer comprises 1-dodecylazacycloheptan-2-one (or Azone). In some embodiments, the penetration enhancer comprises oleoyl polyoxyl-6 glycerides (e.g., Labrafil® M 1944 CS) and 1-dodecylazacycloheptan-2-one (or Azone).

In some embodiments, the penetration enhancer is present in the topical pharmaceutical composition in an amount of about 5 wt % to about 10 wt %. In some embodiments, the penetration enhancer comprises polyglyceryl-3 dioleate (e.g., Plurol® Oleique CC 497). In some embodiments, the penetration enhancer comprises diethylene glycol monoethyl ether (e.g., Transcutol® P). In some embodiments, the penetration enhancer comprises oleoyl polyoxyl-6 glycerides (e.g., Labrafil® M 1944 CS).

In some embodiments, polyglyceryl-3 dioleate (e.g., Plurol® Oleique CC 497) is present in the topical pharmaceutical composition in an amount of about 1 wt % to about 15 wt %. In some embodiments, polyglyceryl-3 dioleate (e.g., Plurol® Oleique CC 497) is present in the topical pharmaceutical composition in an amount of about 1 wt % to about 2 wt %, about 2 wt % to about 5 wt %, about 5 wt % to about 7 wt %, about 7 wt % to about 10 wt %, or about 10 wt % to about 15 wt %. In some embodiments, polyglyceryl-3 dioleate (e.g., Plurol® Oleique CC 497) is present in the topical pharmaceutical composition in an amount of about 1 wt % to about 2 wt %. In some embodiments, polyglyceryl-3 dioleate (e.g., Plurol® Oleique CC 497) is present in the topical pharmaceutical composition in an amount of about 2 wt % to about 5 wt %. In some embodiments, polyglyceryl-3 dioleate (e.g., Plurol® Oleique CC 497) is present in the topical pharmaceutical composition in an amount of about 5 wt % to about 7 wt %. In some embodiments, polyglyceryl-3 dioleate (e.g., Plurol® Oleique CC 497) is present in the topical pharmaceutical composition in an amount of about 7 wt % to about 10 wt %. In some embodiments, polyglyceryl-3 dioleate (e.g., Plurol® Oleique CC 497) is present in the topical pharmaceutical composition in an amount of about 10 wt % to about 15 wt %. In some embodiments, polyglyceryl-3 dioleate (e.g., Plurol® Oleique CC 497) is present in the topical pharmaceutical composition in an amount of about 5 wt %.

### pH Stabilizer

In one aspect, disclosed herein are topical pharmaceutical compositions comprising midostaurin or a pharmaceutically acceptable salt thereof, a carrier vehicle, and an excipient (e.g., a pH stabilizer).

In some embodiments, topical pharmaceutical compositions described herein comprise a pH stabilizer. In some embodiments, gel topical pharmaceutical compositions described herein comprise a pH stabilizer. In some embodiments, the topical pharmaceutical compositions described herein comprise a means for maintaining the pH. In some embodiments, the means for maintaining the pH is one or more pH stabilizers. In some embodiments, the topical pharmaceutical compositions described herein comprise two or more pH stabilizers. In some embodiments, the pH stabilizer comprises an organic or inorganic acid or acids. The pharmaceutically acceptable organic acid is selected from the group consisting of tartaric acid, fumaric acid, succinic acid, citric acid, lactic acid, malic acid, aliphatic sulfonic acids (e.g., methanesulfonic acid, ethanesulfonic acid, isethionic acid, etc.) and aromatic sulfonic acids (e.g., benzenesulfonic acid, p-toluenesulfonic acid, etc.), and the pharmaceutically acceptable inorganic acid is selected from the group consisting of hydrochloric acid, sulfuric acid, phosphoric acid and the like. In some embodiments, the pH stabilizer comprises a salt of an organic or inorganic acid.

In some embodiments, the pH stabilizer comprises citric acid, glycolic acid, lactic acid, sodium hydroxide, sodium bicarbonate. L-arginine, triethanolamine, or a combination thereof. In some embodiments, the pH stabilizer comprises sodium hydroxide, sodium bicarbonate. L-arginine. triethanolamine, or a combination thereof. In some embodiments, the pH stabilizer comprises an amine. In some embodiments, the pH stabilizer comprises triethanolamine. In some embodiments, the pH stabilizer comprises sodium hydroxide. In some embodiments, the topical pharmaceutical compositions are gel formulations.

In some embodiments, the pH stabilizer comprises citric acid, sodium citrate, sodium tartrate. sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate. calcium acetate, calcium glycerophosphate, calcium chloride, calcium hydroxide, calcium lactate. calcium carbonate, calcium bicarbonate, and other calcium salts. Further examples of pH stabilizer include, but are not limited to sodium bicarbonate, potassium bicarbonate, magnesium hydroxide. magnesium lactate, magnesium gluconate, aluminum hydroxide, aluminum hydroxide/sodium bicarbonate co-precipitate, mixture of an amino acid and a buffer, a mixture of aluminum glycinate and a buffer, a mixture of an acid salt of an amino acid and a buffer, and a mixture of an alkali salt of an amino acid and a buffer. In some embodiments, the pH stabilizer comprises citric acid. In some embodiments, the topical pharmaceutical compositions are cream formulations.

In some embodiments, a suitable amount of pH stabilizer is present in the topical pharmaceutical composition described herein to maintain a pH of the composition at from about 5.0 to about 8.0, about 5.5 to about 7.5, about 6.0 to about 7.5, about 6.5 to about 7.5, about 6.7 to about 7.3, about 6.8 to about 7.2, about 6.9 to about 7.1, or about 7.0. In some embodiments, a suitable amount of pH stabilizer is present in the topical pharmaceutical composition described herein to maintain a pH of the composition at from about 5.0 to about 8.0. In some embodiments, a suitable amount of pH stabilizer is present in the topical pharmaceutical composition described herein to maintain a pH of the composition at from about 5.5 to about 7.5. In some embodiments, a suitable amount of pH stabilizer is present in the topical pharmaceutical composition described herein to maintain a pH of the composition at from about 6.0 to about 7.5. In some embodiments, a suitable amount of pH stabilizer is present in the topical pharmaceutical composition described herein to maintain a pH of the composition at from about 6.5 to about 7.5. In some embodiments, a suitable amount of pH stabilizer is present in the topical pharmaceutical composition described herein to maintain a pH of the composition at from about 6.7 to about 7.3. In some embodiments, a suitable amount of pH stabilizer is present in the topical pharmaceutical composition described herein to maintain a pH of the composition at from about 6.8 to about 7.2. In some embodiments, a suitable amount of pH stabilizer is present in the topical pharmaceutical composition described herein to maintain a pH of the composition at from about 6.9 to about 7.1. In some embodiments, a suitable amount of pH stabilizer is present in the topical pharmaceutical composition described herein to maintain a pH of the composition at about 7.0. In some embodiments, the pH stabilizer is triethanolamine.

### Antioxidant

In one aspect, the topical pharmaceutical compositions described herein comprise midostaurin or a pharmaceutically acceptable salt thereof, a carrier vehicle, and an excipient (e.g., a preservative. including antioxidants). In some embodiments, the topical pharmaceutical compositions described herein comprise a preservative. The preservatives can include anti-microbials, antioxidants, and agents that enhance sterility or reduces oxidation. Exemplary preservatives include ascorbic acid, ascorbyl palmitate, butylatedhydroxyanisole (BHA). Butylatedhydroxytoulene (BHT), propyl gallate, citric acid, EDTA and its salts, erythorbic acid, fumaric acid, malic acid, propyl gallate, sodium ascorbate, sodium bisulfate, sodium metabisulfite, sodium sulfite, parabens (such as methylparaben, ethylparaben, propylparaben, butylparaben and their salts), benzoic acid, sodium benzoate, potassium sorbate, vanillin, and the like.

In some embodiments, topical pharmaceutical compositions described herein comprise an antioxidant. In some embodiments, topical pharmaceutical compositions described herein comprise two or more antioxidants. In some embodiments, the antioxidant comprises a-tocopherol acetate, acetone sodium bisulfite, acetylcysteine, ascorbic acid, vitamin E, ascorbyl palmitate, BHA, BHT, cysteine, cysteine hydrochloride, d-a-tocopherol (natural or synthetic), dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea, or tocopherols (e.g., d-a-tocopherol), or a combination thereof.

In some embodiments, the antioxidant is present in the topical pharmaceutical composition in an amount of about 0.0001 wt % to about 15 wt %. In some embodiments, the antioxidant is present in the topical pharmaceutical composition in an amount of about 0.0001 wt % to about 0.001 wt %, about 0.0001 wt % to about 0.01 wt %, about 0.0001 wt % to about 0.1 wt %, about 0.0001 wt % to about 1 wt %, about 0.0001 wt % to about 2 wt %, about 0.0001 wt % to about 5 wt %, about 0.0001 wt % to about 7 wt %, about 0.0001 wt % to about 10 wt %, about 0.0001 wt % to about 15 wt %, about 0.001 wt % to about 0.01 wt %, about 0.001 wt % to about 0.1 wt %, about 0.001 wt % to about 1 wt %, about 0.001 wt % to about 2 wt %, about 0.001 wt % to about 5 wt %, about 0.001 wt % to about 7 wt %, about 0.001 wt % to about 10 wt %, about 0.001 wt % to about 15 wt %, about 0.01 wt % to about 0.1 wt %, about 0.01 wt % to about 1 wt %, about 0.01 wt % to about 2 wt %, about 0.01 wt % to about 5 wt %, about 0.01 wt % to about 7 wt %, about 0.01 wt % to about 10 wt %, about 0.01 wt % to about 15 wt %, about 0.1 wt % to about 1 wt %, about 0.1 wt % to about 2 wt %, about 0.1 wt % to about 5 wt %, about 0.1 wt % to about 7 wt %, about 0.1 wt % to about 10 wt %, about 0.1 wt % to about 15 wt %, about 1 wt % to about 2 wt %, about 1 wt % to about 5 wt %, about 1 wt % to about 7 wt %, about 1 wt % to about 10 wt %, about 1 wt % to about 15 wt %, about 2 wt % to about 5 wt %, about 2 wt % to about 7 wt %, about 2 wt % to about 10 wt %, about 2 wt % to about 15 wt %, about 5 wt % to about 7 wt %, about 5 wt % to about 10 wt %, about 5 wt % to about 15 wt %, about 7 wt % to about 10 wt %, about 7 wt % to about 15 wt %, or about 10 wt % to about 15 wt %. In some embodiments, the antioxidant is present in the topical pharmaceutical composition in an amount of about 0.0001 wt %, about 0.001 wt %, about 0.01 wt %. about 0.1 wt %, about 1 wt %, about 2 wt %, about 5 wt %, about 7 wt %, about 10 wt %, or about 15 wt %. In some embodiments, the antioxidant is present in the topical pharmaceutical composition by weight of at least about 0.0001 wt %, about 0.001 wt %, about 0.01 wt %, about 0.1 wt %, about 1 wt %, about 2 wt %, about 5 wt %, about 7 wt %, or about 10 wt %.

In some embodiments, the antioxidant is present in the topical pharmaceutical composition in an amount of about 0.01 wt % to about 0.1 wt %. In some embodiments, the antioxidant is present in the topical pharmaceutical composition in an amount of about 0.06 wt %. In some embodiments, the antioxidant comprises butylated hydroxytoluene (BHT), butylated hydroxyanisole (BHA) and ascorbyl palmitate.

### Viscosity Modifier

Viscosity modifiers are agents that can increase or decrease viscosity of a pharmaceutical composition or change its texture. Viscosity modifiers can include rheology modifiers, or other names known in the art. Viscosity modifiers can include a viscosity enhancer, viscosity-enhancing agent, a thickening agent, or other names well known in the art. Viscosity modifiers can also include a viscosity reducer, a viscosity-reducing agent, or other names well known in the art. Examples of viscosity modifiers include, but are not limited to, aluminum monostearate, bentonite, polyacrylic acid (or PAA) (e.g., crosslinked polyacrylic acid, sold under the tradename Carbomer), octadecyl alcohol, glyceryl behenate (e.g., sold under the trademark Compritol® 888 ATO), white Vaseline, silicone compounds (e.g., a mixture of cyclopentasiloxane, dimethicone, and phenyl trimethicone, sold under the trademark GELAID®).

In some embodiments, the midostaurin topical pharmaceutical compositions described herein comprise a viscosity modifier. In some embodiments, the viscosity modifier comprises aluminum monostearate, bentonite, polyacrylic acid (or PAA) (e.g., crosslinked polyacrylic acid, sold under the tradename Carbomer), octadecyl alcohol, glyceryl behenate (e.g., Compritol® 888 ATO), white Vaseline, or a combination thereof.

In some embodiments, the viscosity modifier is present in the topical pharmaceutical composition in an amount of about 0.01 wt % to about 15 wt %. In some embodiments, the viscosity modifier is present in the topical pharmaceutical composition in an amount of about 0.01 wt % to about 0.1 wt %, about 0.01 wt % to about 1 wt %, about 0.01 wt % to about 2 wt %, about 0.01 wt % to about 5 wt %, about 0.01 wt % to about 7 wt %, about 0.01 wt % to about 10 wt %, about 0.01 wt % to about 15wt %, about 0.1 wt % to about 0.5 wt %, about 0.1 wt % to about 1 wt %, about 0.1 wt % to about 2 wt %, about 0.1 wt % to about 5 wt %, about 0.1 wt % to about 7 wt %, about 0.1 wt % to about 10 wt %, about 0.1 wt % to about 15 wt %, about 0.5 wt % to about 1 wt %, about 0.5 wt % to about 2 wt %, about 0.5 wt % to about 5 wt %, about 0.5 wt % to about 7 wt %, about 0.5 wt % to about 10 wt %, about 0.5 wt % to about 15 wt %, about 1 wt % to about 2 wt %, about 1 wt % to about 5 wt %, about 1 wt % to about 7 wt %, about 1 wt % to about 10 wt %, about 1 wt % to about 15 wt %, about 2 wt % to about 5 wt %, about 2 wt % to about 7 wt %, about 2 wt % to about 10 wt %, about 2 wt % to about 15 wt %, about 5 wt % to about 7 wt %, about 5 wt % to about 10 wt %, about 5 wt % to about 15 wt %, about 7 wt % to about 10 wt %, about 7 wt % to about 15 wt %, or about 10 wt % to about 15 wt %. In some embodiments, the viscosity modifier is present in the topical pharmaceutical composition in an amount of about 0.5 wt %, about 1 wt %, about 2 wt %, about 3 wt %, about 3.5 wt %, or about 5 wt %. In some embodiments, the viscosity modifier is present in the topical pharmaceutical composition in an amount of about 0.5 wt %. In some embodiments, the viscosity modifier is present in the topical pharmaceutical composition in an amount of about 3 wt %. In some embodiments, the viscosity modifier is present in the topical pharmaceutical composition in an amount of about 3.5 wt %. In some embodiments, the viscosity modifier comprises aluminum monostearate, bentonite, polyacrylic acid (or PAA) (e.g., crosslinked polyacrylic acid, or Carbomer), octadecyl alcohol, petroleum jelly (e.g., white Vaseline), or a combination thereof.

In some embodiments, crosslinked polyacrylic acid (or Carbomer) is present in the topical pharmaceutical composition in an amount of about 0.1 wt % to about 1 wt %. In some embodiments, crosslinked polyacrylic acid (or Carbomer) is present in the topical pharmaceutical composition in an amount of about 0.25 wt % to about 0.75 wt %. In some embodiments, crosslinked polyacrylic acid (or Carbomer) is present in the topical pharmaceutical composition in an amount of about 0.5 wt %.

In some embodiments, octadecyl alcohol is present in the topical pharmaceutical composition in an amount of about 1 wt % to about 5 wt %. In some embodiments, octadecyl alcohol is present in the topical pharmaceutical composition in an amount of about 2 wt % to about 4 wt %. In some embodiments, octadecyl alcohol is present in the topical pharmaceutical composition in an amount of about 3.5 wt %.

### Moisturizer

Moisturizers are agents which bind water or moisture to skin or retain moisture in skin.

Moisturizers can include humectants, which can bind water or moisture to skin. Moisturizers can include occlusives, which can create a protective barrier that retains moisture in skin and prevents water loss. Moisturizers can include emollient, which keeps skin hydrated and smooth. Examples of moisturizers include, but are not limited to, petrolatum, mineral oil, wax, liquid paraffin, petroleum jelly (e.g., white Vaseline), glycerol (or glycerin), propylene glycol, polyethylene glycol (PEG), hexylene glycol, and sorbitol. (Sec, for example, US Publication No. US 2004/0191276, herein incorporated by reference).

In some embodiments, the midostaurin topical pharmaceutical compositions described herein comprise a moisturizer. In some embodiments, the midostaurin topical pharmaceutical compositions described herein comprise two or more moisturizers. In some embodiments, the moisturizer comprises petrolatum, mineral oil, wax, liquid paraffin, petroleum jelly, glycerol (or glycerin), propylene glycol. hexylene glycol, or a combination thereof.

In some embodiments, a moisturizer is present in the topical pharmaceutical composition in an amount of about 0.01 wt % to about 35 wt %. In some embodiments, the moisturizer is present in the topical pharmaceutical composition in an amount of about 0.01 wt % to about 1 wt %, about 1 wt % to about 5 wt %, about 5 wt % to about 10 wt %, about 10 wt % to about 15 wt %, about 15 wt % to about 20 wt %, about 20 wt % to about 25 wt %, about 25 wt % to about 30 wt %, or about 30 wt % to about 35 wt %. In some embodiments, the moisturizer is present in the topical pharmaceutical composition in an amount of about 0.01 wt % to about 1 wt %. In some embodiments, the moisturizer is present in the topical pharmaceutical composition in an amount of about 1 wt % to about 5 wt %. In some embodiments, the moisturizer is present in the topical pharmaceutical composition in an amount of about 5 wt % to about 10 wt %. In some embodiments, the moisturizer is present in the topical pharmaceutical composition in an amount of about 10 wt % to about 15 wt %. In some embodiments, the moisturizer is present in the topical pharmaceutical composition in an amount of about 20 wt % to about 25 wt %. In some embodiments, the moisturizer is present in the topical pharmaceutical composition in an amount of about 25 wt % to about 30 wt %. In some embodiments, the moisturizer is present in the topical pharmaceutical composition in an amount of about 30 wt % to about 35 wt %. In some embodiments, the moisturizer comprises liquid paraffin, petroleum jelly (e.g., white Vaseline), glycerol (or glycerin). propylene glycol, or a combination thereof.

In some embodiments, the moisturizer comprises glycerol. In some embodiments, the glycerol is present in the topical pharmaceutical composition in an amount of about 5 wt % to about 10 wt %. In some embodiments, the glycerol is present in the topical pharmaceutical composition in an amount of about 10) wt % to about 15 wt %. In some embodiments, the glycerol is present in the topical pharmaceutical composition in an amount of about 5 wt %. In some embodiments, the glycerol is present in the topical pharmaceutical composition in an amount of about 13 wt %.

In some embodiments, the moisturizer comprises glycerol (or glycerin) and propylene glycol. In some embodiments, the moisturizer is present in the topical pharmaceutical composition in an amount of about 10 wt % to about 30 wt %. In some embodiments, the moisturizer is present in the topical pharmaceutical composition in an amount of about 15 wt % to about 25 wt %. In some embodiments, the moisturizer is present in the topical pharmaceutical composition in an amount of about 20 wt %.

### Sun-Screening Agent

Sun-screening are agents that can protect skin from ultraviolet (UV) radiation. Sun-screening agents can absorb UV and visible sun rays. Sun-screening agents can also reflect, scatter, or block UV and visible sun rays. Sun-screening agents can include a physical sun-screening agent. Sun-screening agents can include a chemical sun-screening agent. Other terms in the art include sun-screening agent, sunscreen, sunblock and the like. Non-limiting examples of sun-screening agent include avobenzone, bemotrizinol, benzophenone-3 (BZ-3, oxy benzone), bisoctizole, homosalate, octinoxate, octisalate, octocrylene, oxybenzone, titanium dioxide, or zinc oxide, or a combination thereof. Other examples of sun-screening agents are described in Latha M S, Martis J, Shobha V, et al. Sunscreening agents; a review. J Clin Aesthet Dermatol. 2013; 6 (1); 16-26, herein incorporated by reference.

In some embodiments, the midostaurin topical pharmaceutical compositions described herein

comprise a sun-screening agent. In some embodiments, the sun-screening agent is present in the topical pharmaceutical composition in an amount of about 0.0001 wt % to about 5 wt %. In some embodiments, the sun-screening agent is present in the topical pharmaceutical composition in an amount of about 0.0001 wt % to about 0.001 wt %, about 0.0001 wt % to about 0.01 wt %, about 0.0001 wt % to about 0.1 wt %, about 0.0001 wt % to about 1 wt %, about 0.0001 wt % to about 2 wt %, about 0.0001 wt % to about 5 wt %, about 0.001 wt % to about 0.01 wt %, about 0.001 wt % to about 0.1 wt %, about 0.001 wt % to about 1 wt %, about 0.001 wt % to about 2 wt %, about 0.001 wt % to about 5 wt %, about 0.01 wt % to about 0.1 wt %, about 0.01 wt % to about 1 wt %, about 0.01 wt % to about 2 wt %, about 0.01 wt % to about 5 wt %, about 0.1 wt % to about 1 wt %, about 0.1 wt % to about 2 wt %, about 0.1 wt % to about 5 wt %, about 1 wt % to about 2 wt %, about 1 wt % to about 5 wt %, about 2 wt % to about 5 wt %, or about 3 wt % to about 5 wt % In some embodiments, the sun-screening agent is present in the topical pharmaceutical composition in an amount of from about 0.001 wt % to about 0.01 wt %. In some embodiments, the sun-screening agent is present in the topical pharmaceutical composition in an amount of from about 0.01 wt % to about 0.1 wt %. In some embodiments, the sun-screening agent is present in the topical pharmaceutical composition in an amount of from about 0.1 wt % to about 0.5 wt %. In some embodiments, the sun-screening agent is present in the topical pharmaceutical composition in an amount of from about 0.5 wt % to about 1 wt %. In some embodiments, the sun-screening agent is present in the topical pharmaceutical composition in an amount of from about 1 wt % to about 2 wt %. In some embodiments, the sun-screening agent comprises avobenzone, bemotrizinol, benzophenone-3 (BZ-3, oxybenzone), bisoctizole, homosalate, octinoxate, octisalate, octocrylene, oxybenzone, titanium dioxide, or zinc oxide, or a combination thereof.

### Other Additives

The excipients described herein can be used alone or in combination. In some embodiments, other additives conventionally mixed with pharmaceutical compositions can be included the topical pharmaceutical composition. The additives include, but are not limited to, a suspending agent (e.g., cellulose ether), an anti-foaming agent (e.g., hydrophobic silica), or any suitable agents that can improve skin conditions in general, such as alpha hydroxy acids (e.g., glycolic acid and lactic acids).

Additives can also be materials such as proteins (e.g., collagen, gelatin. Zein, gluten, mussel protein, lipoprotein), carbohydrates (e.g., alginates, carrageenan, cellulose derivatives, pectin, starch, chitosan), gums (e.g., xanthan gum, gum arabic), spermaceti, natural or synthetic waxes, carnauba wax, fatty acids (e.g., stearic acid, hydroxystearic acid), fatty alcohols, sugars, shellacs, such as those based on sugars (e.g., lactose, sucrose, dextrose) or starches, polysaccharide-based polymers (e.g., maltodextrin and maltodextrin derivatives, dextrates, cyclodextrin and cyclodextrin derivatives), cellulosic-based polymers (e.g., microcrystalline cellulose, sodium carboxymethyl cellulose, hydroxy propylmethyl cellulose, ethyl cellulose, hydroxypropyl cellulose, cellulose acetate, cellulose nitrate, cellulose acetate butyrate, cellulose acetate, trimellitate, carboxymethylethyl cellulose, hydroxypropylmethyl cellulose phthalate), inorganics. (e.g., dicalcium phosphate, hydroxyapitite, tricalcium phosphate, talc and titania), polyols (e.g., mannitol, xylitol and sorbitol polyethylene glycol esters) and polymers (e.g., alginates, poly (lactide coglycolide), gelatin, crosslinked gelatin and agar-agar). In some embodiments, the proteins comprise amino acids such as glutamic acid, aspartic acid, or acidic salts of glycine, alanine or serine.

### Stability

In some embodiments, topical pharmaceutical compositions described herein are stable in various storage conditions including refrigerated conditions, ambient conditions, room temperature, and accelerated conditions. In some embodiments, a stable midostaurin composition as used herein refer to midostaurin topical pharmaceutical compositions having about 95% or greater of the initial midostaurin amount and about 5 wt % or less total impurities or related substances at the end of a given storage period. In some embodiments, a stable midostaurin composition as used herein refer to midostaurin topical pharmaceutical compositions having about 90 wt % or greater of initial midostaurin amount and about 10 wt % or less total impurities or related substances at the end of a given storage period.

The percentage of impurities can be calculated from the amount of impurities relative to the amount of midostaurin. The percentage of impurities can be assessed by high-performance liquid chromatography (HPLC), such as using the method described in Table B-2, or any other known testing method. In some embodiments, a stable midostaurin topical pharmaceutical composition contains no more than about 10 wt %, about 5 wt %, about 4 wt %, about 3 wt %, about 2.5 wt %, about 2 wt %, about 1.5 wt %, about 1 wt %, or about 0.5 wt % total impurities or related substances. In other embodiments, a stable midostaurin topical pharmaceutical composition contains no more than about 10 wt % total impurities or related substances. In other embodiments, a stable midostaurin topical pharmaceutical composition contains no more than about 5 wt % total impurities or related substances. In yet other embodiments, a stable midostaurin topical pharmaceutical composition contains no more than about 4 wt % total impurities or related substances. In yet other embodiments, a stable midostaurin topical pharmaceutical composition contains no more than about 3 wt % total impurities or related substances. In yet other embodiments, a stable midostaurin topical pharmaceutical composition contains no more than about 2 wt % total impurities or related substances. In yet other embodiments, a stable midostaurin topical pharmaceutical composition contains no more than about 1 wt % total impurities or related substances.

The percentage of midostaurin retained in a composition can be calculated from the amount of midostaurin in the composition at a certain time point relative to the initial amount of midostaurin. Assay or midostaurin content can be assessed by HPLC, such as using the method described in Table A-1, or any other known testing method. In some embodiments, a stable midostaurin topical pharmaceutical composition retains at least about 90 wt %, about 91 wt %, about 92 wt %, about 93 wt %, about 94 wt %, about 95 wt %, about 96 wt %, about 97 wt %, about 98 wt %, about 99 wt %, about 99.5 wt %, or about 99.9 wt % of the initial midostaurin amount. In yet other embodiments, a stable midostaurin topical pharmaceutical composition retains at least about 90 wt % of the initial midostaurin amount. In yet other embodiments, a stable midostaurin topical pharmaceutical composition retains at least about 91wt % of the initial midostaurin amount. In yet other embodiments, a stable midostaurin topical pharmaceutical composition retains at least about 92 wt % of the initial midostaurin amount. In yet other embodiments, a stable midostaurin topical pharmaceutical composition retains at least about 93 wt % of the initial midostaurin amount. In yet other embodiments, a stable midostaurin topical pharmaceutical composition retains at least about 94wt % of the initial midostaurin amount. In yet other embodiments, a stable midostaurin topical pharmaceutical composition retains at least about 95 wt % of the initial midostaurin amount. In yet other embodiments, a stable midostaurin topical pharmaceutical composition retains at least about 96 wt % of the initial midostaurin amount. In yet other embodiments, a stable midostaurin topical pharmaceutical composition retains at least about 97 wt % of the initial midostaurin amount. In yet other embodiments, a stable midostaurin topical pharmaceutical composition retains at least about 98 wt % of the initial midostaurin amount. In yet other embodiments, a stable midostaurin topical pharmaceutical composition retains at least about 99 wt % of the initial midostaurin amount. In yet other embodiments, a stable midostaurin topical pharmaceutical composition retains at least about 99.5 wt % of the initial midostaurin amount. In yet other embodiments, a stable midostaurin topical pharmaceutical composition retains at least about 99.8 wt % of the initial midostaurin amount.

In some embodiments, midostaurin topical pharmaceutical compositions described herein are stable for at least 1 day, 3 days, 7 days, 14 days, 21 days, or 30 days under the condition of 4500 lux (lx) in light exposure. In some embodiments, midostaurin topical pharmaceutical compositions are stable for at least 1 day under the condition of 4500 lux (lx) in light exposure. In some embodiments, midostaurin topical pharmaceutical compositions are stable for at least 3 days under the condition of 4500 lux (lx) in light exposure. In some embodiments, midostaurin topical pharmaceutical compositions are stable for at least 7 days under the condition of 4500 lux (lx) in light exposure. In some embodiments, midostaurin topical pharmaceutical compositions are stable for at least 21 days under the condition of 4500 lux (lx) in light exposure. In some embodiments, midostaurin topical pharmaceutical compositions are stable for at least 30 days under the condition of 4500 lux (lx) in light exposure.

In some embodiments, midostaurin topical pharmaceutical compositions described herein are stable for at least 1 day, 3 days, 7 days, 14 days, 21 days, or 30 days under the condition of 4500 lux (lx) in light exposure and about 15° C. to about 25° C. in temperature. In some embodiments, midostaurin topical pharmaceutical compositions are stable for at least 1 day under the condition of 4500 lux (lx) in light exposure and about 15° C. to about 25° C. in temperature. In some embodiments, midostaurin topical pharmaceutical compositions are stable for at least 3 days under the condition of 4500 lux (lx) in light exposure and about 15° C. to about 25° C. in temperature. In some embodiments, midostaurin topical pharmaceutical compositions are stable for at least 7 days under the condition of 4500 lux (lx) in light exposure and about 15° C. to about 25° C. in temperature. In some embodiments, midostaurin topical pharmaceutical compositions are stable for at least 21 days under the condition of 4500 lux (lx) in light exposure and about 15° C. to about 25° C. in temperature. In some embodiments, midostaurin topical pharmaceutical compositions are stable for at least 30 days under the condition of 4500 lux (lx) in light exposure and about 15° C. to about 25° C. in temperature.

In some embodiments, midostaurin topical pharmaceutical compositions described herein are stable for at least 1 day, 3 days, 7 days, 14 days, or 21 days when stored at about 60° C. In some embodiments, midostaurin topical pharmaceutical compositions are stable for at least 1 day when stored at about 60° C. In some embodiments, midostaurin topical pharmaceutical compositions are stable for at least 3 days when stored at about 60° C. In some embodiments, midostaurin topical pharmaceutical compositions are stable for at least 7 days when stored at about 60° C. In some embodiments, midostaurin topical pharmaceutical compositions are stable for at least 21 days when stored at about 60° C.

In some embodiments, midostaurin topical pharmaceutical compositions described herein are stable for at least 1 day, 3 days, 7 days, 14 days, 21 days, or 30 days after at least one freeze thaw cycle. In some embodiments, a stable topical pharmaceutical composition is chemically and physically stable. In some embodiments, midostaurin topical pharmaceutical compositions described herein are physically stable after one or more freeze thaw cycles. In some embodiments, midostaurin topical pharmaceutical compositions are stable for at least 1 day after at least one freeze thaw cycle. In some embodiments, midostaurin topical pharmaceutical compositions are stable for at least 3 days after at least one freeze thaw cycle. In some embodiments, midostaurin topical pharmaceutical compositions are stable for at least 7 days after at least one freeze thaw cycle. In some embodiments, midostaurin topical pharmaceutical compositions are stable for at least 21 days after at least one freeze thaw cycle. In some embodiments, midostaurin topical pharmaceutical compositions are stable for at least 30 days after at least one freeze thaw cycle.

In some embodiments, midostaurin topical formulations described herein are stable when stored at refrigerated conditions for at least 1 month, at least 2 months, at least 3 months, at least 6 months, at least 9 months, at least 12 months, at least 15 months, at least 18 months, at least 24 months, at least 30 months, or at least 36 months. In some embodiments, midostaurin topical formulations described herein are stable when stored at refrigerated conditions for at least 1 month. In some embodiments, midostaurin topical formulations described herein are stable when stored at refrigerated conditions for at least 3 months. In some embodiments, midostaurin topical formulations described herein are stable when stored at refrigerated conditions for at least 6 months. In some embodiments, midostaurin topical formulations described herein are stable when stored at refrigerated conditions for at least 9 months. In some embodiments, midostaurin topical formulations described herein are stable when stored at refrigerated conditions for at least 12 months. In some embodiments, midostaurin topical formulations described herein are stable when stored at refrigerated conditions for at least 15 months. In some embodiments, midostaurin topical formulations described herein are stable when stored at refrigerated conditions for at least 18 months. In some embodiments, midostaurin topical formulations described herein are stable when stored at refrigerated conditions for at least 24 months. In some embodiments, midostaurin topical formulations described herein are stable when stored at refrigerated conditions for at least 30 months. In some embodiments, midostaurin topical formulations described herein are stable when stored at refrigerated conditions for at least 36 months.

In some embodiments, midostaurin topical formulations described herein retains at least 90 wt % of midostaurin or the pharmaceutically acceptable salt thereof after stored at refrigerated conditions for at least 1 month, at least 2 months, at least 3 months, at least 6 months, at least 9 months, at least 12 months, at least 15 months, at least 18 months, at least 24 months, at least 30 months, or at least 36 months. In some embodiments, midostaurin topical formulations retains at least 90 wt % of midostaurin or the pharmaceutically acceptable salt thereof after stored at refrigerated conditions for at least 1 month. In some embodiments, midostaurin topical formulations retains at least 90 wt % of midostaurin or the pharmaceutically acceptable salt thereof after stored at refrigerated conditions for at least 3 months. In some embodiments, midostaurin topical formulations retains at least 90 wt % of midostaurin or the pharmaceutically acceptable salt thereof after stored at refrigerated conditions for at least 6 months. In some embodiments, midostaurin topical formulations retains at least 90 wt % of midostaurin or the pharmaceutically acceptable salt thereof after stored at refrigerated conditions for at least 9 months. In some embodiments, midostaurin topical formulations retains at least 90 wt % of midostaurin or the pharmaceutically acceptable salt thereof after stored at refrigerated conditions for at least 12 months. In some embodiments, midostaurin topical formulations retains at least 90 wt % of midostaurin or the pharmaceutically acceptable salt thereof after stored at refrigerated conditions for at least 15 months. In some embodiments, midostaurin topical formulations retains at least 90 wt % of midostaurin or the pharmaceutically acceptable salt thereof after stored at refrigerated conditions for at least 18 months. In some embodiments, midostaurin topical formulations retains at least 90 wt % of midostaurin or the pharmaceutically acceptable salt thereof after stored at refrigerated conditions for at least 24 months. In some embodiments, midostaurin topical formulations retains at least 90 wt % of midostaurin or the pharmaceutically acceptable salt thereof after stored at refrigerated conditions for at least 30 months. In some embodiments, midostaurin topical formulations retains at least 90 wt % of midostaurin or the pharmaceutically acceptable salt thereof after stored at refrigerated conditions for at least 36 months.

In some embodiments, midostaurin topical formulations described herein contains no more than 5 wt % of total impurity after stored at refrigerated conditions for at least 1 month, at least 2 months, at least 3 months, at least 6 months, at least 9 months, at least 12 months, at least 15 months, at least 18 months, at least 24 months, at least 30 months, or at least 36 months. In some embodiments, midostaurin topical formulations contains no more than 5 wt % of total impurity after stored at refrigerated conditions for at least 1 month. In some embodiments, midostaurin topical formulations contains no more than 5 wt % of total impurity after stored at refrigerated conditions for at least 3 months. In some embodiments, midostaurin topical formulations contains no more than 5 wt % of total impurity after stored at refrigerated conditions for at least 6 months. In some embodiments, midostaurin topical formulations contains no more than 5 wt % of total impurity after stored at refrigerated conditions for at least 9 months. In some embodiments, midostaurin topical formulations contains no more than 5 wt % of total impurity after stored at refrigerated conditions for at least 12 months. In some embodiments, midostaurin topical formulations contains no more than 5 wt % of total impurity after stored at refrigerated conditions for at least 15 months. In some embodiments, midostaurin topical formulations contains no more than 5 wt % of total impurity after stored at refrigerated conditions for at least 18 months. In some embodiments, midostaurin topical formulations contains no more than 5 wt % of total impurity after stored at refrigerated conditions for at least 24 months. In some embodiments, midostaurin topical formulations contains no more than 5 wt % of total impurity after stored at refrigerated conditions for at least 30 months. In some embodiments, midostaurin topical formulations contains no more than 5 wt % of total impurity after stored at refrigerated conditions for at least 36 months.

In some embodiments, midostaurin topical formulations described herein retains at least 95 wt % of midostaurin or the pharmaceutically acceptable salt thereof after stored at refrigerated conditions for at least 1 month, at least 2 months, at least 3 months, at least 6 months, at least 9 months, at least 12 months, at least 15 months, at least 18 months, at least 24 months, at least 30 months, or at least 36 months. In some embodiments, midostaurin topical formulations retains at least 95 wt % of midostaurin or the pharmaceutically acceptable salt thereof after stored at refrigerated conditions for at least 1 month. In some embodiments, midostaurin topical formulations retains at least 95 wt % of midostaurin or the pharmaceutically acceptable salt thereof after stored at refrigerated conditions for at least 3 months. In some embodiments, midostaurin topical formulations retains at least 95 wt % of midostaurin or the pharmaceutically acceptable salt thereof after stored at refrigerated conditions for at least 6 months. In some embodiments, midostaurin topical formulations retains at least 95 wt % of midostaurin or the pharmaceutically acceptable salt thereof after stored at refrigerated conditions for at least 9 months. In some embodiments, midostaurin topical formulations retains at least 95 wt % of midostaurin or the pharmaceutically acceptable salt thereof after stored at refrigerated conditions for at least 12 months. In some embodiments, midostaurin topical formulations retains at least 95 wt % of midostaurin or the pharmaceutically acceptable salt thereof after stored at refrigerated conditions for at least 15 months. In some embodiments, midostaurin topical formulations retains at least 95 wt % of midostaurin or the pharmaceutically acceptable salt thereof after stored at refrigerated conditions for at least 18 months. In some embodiments, midostaurin topical formulations retains at least 95 wt % of midostaurin or the pharmaceutically acceptable salt thereof after stored at refrigerated conditions for at least 24 months. In some embodiments, midostaurin topical formulations retains at least 95 wt % of midostaurin or the pharmaceutically acceptable salt thereof after stored at refrigerated conditions for at least 30 months. In some embodiments, midostaurin topical formulations retains at least 95 wt % of midostaurin or the pharmaceutically acceptable salt thereof after stored at refrigerated conditions for at least 36 months.

In some embodiments, midostaurin topical formulations described herein contains no more than 2 wt % of total impurity after stored at refrigerated conditions for at least 1 month, at least 2 months, at least 3 months, at least 6 months, at least 9 months, at least 12 months, at least 15 months, at least 18 months, at least 24 months, at least 30 months, or at least 36 months. In some embodiments, midostaurin topical formulations contains no more than 2 wt % of total impurity after stored at refrigerated conditions for at least 1 month. In some embodiments, midostaurin topical formulations contains no more than 2 wt % of total impurity after stored at refrigerated conditions for at least 3 months. In some embodiments, midostaurin topical formulations contains no more than 2 wt % of total impurity after stored at refrigerated conditions for at least 6 months. In some embodiments, midostaurin topical formulations contains no more than 2 wt % of total impurity after stored at refrigerated conditions for at least 9 months. In some embodiments, midostaurin topical formulations contains no more than 2 wt % of total impurity after stored at refrigerated conditions for at least 12 months. In some embodiments, midostaurin topical formulations contains no more than 2 wt % of total impurity after stored at refrigerated conditions for at least 15 months. In some embodiments, midostaurin topical formulations contains no more than 2 wt % of total impurity after stored at refrigerated conditions for at least 18 months. In some embodiments, midostaurin topical formulations contains no more than 2 wt % of total impurity after stored at refrigerated conditions for at least 24 months. In some embodiments, midostaurin topical formulations contains no more than 2 wt % of total impurity after stored at refrigerated conditions for at least 30 months. In some embodiments, midostaurin topical formulations contains no more than 2 wt % of total impurity after stored at refrigerated conditions for at least 36 months.

In some embodiments, midostaurin topical formulations described herein are stable when stored at ambient conditions for at least 1 month, at least 2 months, at least 3 months, at least 6 months, at least 9 months, at least 12 months, at least 15 months, at least 18 months, or at least 24 months. In some embodiments, midostaurin topical formulations described herein are stable when stored at ambient conditions for at least 1 month. In some embodiments, midostaurin topical formulations described herein are stable when stored at ambient conditions for at least 3 months. In some embodiments, midostaurin topical formulations described herein are stable when stored at ambient conditions for at least 6 months. In some embodiments, midostaurin topical formulations described herein are stable when stored at ambient conditions for at least 9 months. In some embodiments, midostaurin topical formulations described herein are stable when stored at ambient conditions for at least 12 months. In some embodiments, midostaurin topical formulations described herein are stable when stored at ambient conditions for at least 15 months. In some embodiments, midostaurin topical formulations described herein are stable when stored at ambient conditions for at least 18 months. In some embodiments, midostaurin topical formulations described herein are stable when stored at ambient conditions for at least 24 months.

In some embodiments, midostaurin topical formulations described herein retains at least 90 wt % of midostaurin or the pharmaceutically acceptable salt thereof after stored at ambient conditions for at least 1 month, at least 2 months, at least 3 months, at least 6 months, at least 9 months, at least 12 months, at least 15 months, at least 18 months, or at least 24 months. In some embodiments, midostaurin topical formulations retains at least 90 wt % of midostaurin or the pharmaceutically acceptable salt thereof after stored at ambient conditions for at least 1 month. In some embodiments, midostaurin topical formulations retains at least 90 wt % of midostaurin or the pharmaceutically acceptable salt thereof after stored at ambient conditions for at least 3 months. In some embodiments, midostaurin topical formulations retains at least 90 wt % of midostaurin or the pharmaceutically acceptable salt thereof after stored at ambient conditions for at least 6 months. In some embodiments, midostaurin topical formulations retains at least 90 wt % of midostaurin or the pharmaceutically acceptable salt thereof after stored at ambient conditions for at least 9 months. In some embodiments, midostaurin topical formulations retains at least 90 wt % of midostaurin or the pharmaceutically acceptable salt thereof after stored at ambient conditions for at least 12 months. In some embodiments, midostaurin topical formulations retains at least 90 wt % of midostaurin or the pharmaceutically acceptable salt thereof after stored at ambient conditions for at least 15 months. In some embodiments, midostaurin topical formulations retains at least 90 wt % of midostaurin or the pharmaceutically acceptable salt thereof after stored at ambient conditions for at least 18 months. In some embodiments, midostaurin topical formulations retains at least 90 wt % of midostaurin or the pharmaceutically acceptable salt thereof after stored at ambient conditions for at least 24 months.

In some embodiments, midostaurin topical formulations described herein contains no more than 5 wt % of total impurity after stored at ambient conditions for at least 1 month, at least 2 months, at least 3 months, at least 6 months, at least 9 months, at least 12 months, at least 15 months, at least 18 months. or at least 24 months. In some embodiments, midostaurin topical formulations contains no more than 5 wt % of total impurity after stored at ambient conditions for at least 1 month. In some embodiments, midostaurin topical formulations contains no more than 5 wt % of total impurity after stored at ambient conditions for at least 3 months. In some embodiments, midostaurin topical formulations contains no more than 5 wt % of total impurity after stored at ambient conditions for at least 6 months. In some embodiments, midostaurin topical formulations contains no more than 5 wt % of total impurity after stored at ambient conditions for at least 9 months. In some embodiments, midostaurin topical formulations contains no more than 5 wt % of total impurity after stored at ambient conditions for at least 12 months. In some embodiments, midostaurin topical formulations contains no more than 5 wt % of total impurity after stored at ambient conditions for at least 15 months. In some embodiments, midostaurin topical formulations contains no more than 5 wt % of total impurity after stored at ambient conditions for at least 18 months. In some embodiments, midostaurin topical formulations contains no more than 5 wt % of total impurity after stored at ambient conditions for at least 24 months.

In some embodiments, midostaurin topical formulations described herein retains at least 95 wt % of midostaurin or the pharmaceutically acceptable salt thereof after stored at ambient conditions for at least 1 month, at least 2 months, at least 3 months, at least 6 months, at least 9 months, at least 12 months, at least 15 months, at least 18 months, or at least 24 months. In some embodiments, midostaurin topical formulations retains at least 95 wt % of midostaurin or the pharmaceutically acceptable salt thereof after stored at ambient conditions for at least 1 month. In some embodiments, midostaurin topical formulations retains at least 95 wt % of midostaurin or the pharmaceutically acceptable salt thereof after stored at ambient conditions for at least 3 months. In some embodiments, midostaurin topical formulations retains at least 95 wt % of midostaurin or the pharmaceutically acceptable salt thereof after stored at ambient conditions for at least 6 months. In some embodiments, midostaurin topical formulations retains at least 95 wt % of midostaurin or the pharmaceutically acceptable salt thereof after stored at ambient conditions for at least 9 months. In some embodiments, midostaurin topical formulations retains at least 95 wt % of midostaurin or the pharmaceutically acceptable salt thereof after stored at ambient conditions for at least 12 months. In some embodiments, midostaurin topical formulations retains at least 95 wt % of midostaurin or the pharmaceutically acceptable salt thereof after stored at ambient conditions for at least 15 months. In some embodiments, midostaurin topical formulations retains at least 95 wt % of midostaurin or the pharmaceutically acceptable salt thereof after stored at ambient conditions for at least 18 months. In some embodiments, midostaurin topical formulations retains at least 95 wt % of midostaurin or the pharmaceutically acceptable salt thereof after stored at ambient conditions for at least 24 months.

In some embodiments, midostaurin topical formulations described herein contains no more than 2 wt % of total impurity after stored at ambient conditions for at least 1 month, at least 2 months, at least 3 months, at least 6 months, at least 9 months, at least 12 months, at least 15 months, at least 18 months. or at least 24 months. In some embodiments, midostaurin topical formulations contains no more than 2 wt % of total impurity after stored at ambient conditions for at least 1 month. In some embodiments, midostaurin topical formulations contains no more than 2 wt % of total impurity after stored at ambient conditions for at least 3 months. In some embodiments, midostaurin topical formulations contains no more than 2 wt % of total impurity after stored at ambient conditions for at least 6 months. In some embodiments, midostaurin topical formulations contains no more than 2 wt % of total impurity after stored at ambient conditions for at least 9 months. In some embodiments, midostaurin topical formulations contains no more than 2 wt % of total impurity after stored at ambient conditions for at least 12 months. In some embodiments, midostaurin topical formulations contains no more than 2 wt % of total impurity after stored at ambient conditions for at least 15 months. In some embodiments, midostaurin topical formulations contains no more than 2 wt % of total impurity after stored at ambient conditions for at least 18 months. In some embodiments, midostaurin topical formulations contains no more than 2 wt % of total impurity after stored at ambient conditions for at least 24 months.

In some embodiments, midostaurin topical formulations described herein are stable when stored at about 40° C, for at least 1 month, at least 2 months, at least 3 months, or at least 6 months. In some embodiments, midostaurin topical formulations described herein are stable when stored at about 40° C. for at least 1 month. In some embodiments, midostaurin topical formulations described herein are stable when stored at about 40° C, for at least 2 months. In some embodiments, midostaurin topical formulations described herein are stable when stored at about 40° C. for at least 3 months. In some embodiments, midostaurin topical formulations described herein are stable when stored at about 40° C. for at least 6 months.

In some embodiments, midostaurin topical formulations described herein retains at least 90 wt % of midostaurin or the pharmaceutically acceptable salt thereof after stored at about 40° C. for at least 1 month, at least 2 months, at least 3 months, or at least 6 months. In some embodiments, midostaurin topical formulations retains at least 90 wt % of midostaurin or the pharmaceutically acceptable salt thereof after stored at about 40° C. for at least 1 month. In some embodiments, midostaurin topical formulations retains at least 90 wt % of midostaurin or the pharmaceutically acceptable salt thereof after stored at about 40° C. for at least 2 months. In some embodiments, midostaurin topical formulations retains at least 90 wt % of midostaurin or the pharmaceutically acceptable salt thereof after stored at about 40° C. for at least 3 months. In some embodiments, midostaurin topical formulations retains at least 90 wt % of midostaurin or the pharmaceutically acceptable salt thereof after stored at about 40° C. for at least 6 months.

In some embodiments, midostaurin topical formulations described herein contains no more than 5 wt % of total impurity after stored at about 40° C. for at least 1 month, at least 2 months, at least 3 months, or at least 6 months. In some embodiments, midostaurin topical formulations contains no more than 5 wt % of total impurity after stored at about 40° C. for at least 1 month. In some embodiments, midostaurin topical formulations contains no more than 5 wt % of total impurity after stored at about 40° C. for at least 2 months. In some embodiments, midostaurin topical formulations contains no more than 5 wt % of total impurity after stored at about 40° C. for at least 3 months. In some embodiments, midostaurin topical formulations contains no more than 5 wt % of total impurity after stored at about 40° C. for at least 6 months.

In some embodiments, midostaurin topical formulations described herein retains at least 95 wt % of midostaurin or the pharmaceutically acceptable salt thereof after stored at about 40° C. for at least 1 month, at least 2 months, at least 3 months, or at least 6 months. In some embodiments, midostaurin topical formulations retains at least 95 wt % of midostaurin or the pharmaceutically acceptable salt thereof after stored at about 40° C. for at least 1 month. In some embodiments, midostaurin topical formulations retains at least 95 wt % of midostaurin or the pharmaceutically acceptable salt thereof after stored at about 40° C. for at least 2 months. In some embodiments, midostaurin topical formulations retains at least 95 wt % of midostaurin or the pharmaceutically acceptable salt thereof after stored at about 40° C. for at least 3 months. In some embodiments, midostaurin topical formulations retains at least 95 wt % of midostaurin or the pharmaceutically acceptable salt thereof after stored at about 40° C. for at least 6 months.

In some embodiments, midostaurin topical formulations described herein contains no more than 2 wt % of total impurity after stored at about 40° C. for at least 1 month, at least 2 months, at least 3 months, or at least 6 months. In some embodiments, midostaurin topical formulations contains no more than 2 wt % of total impurity after stored at about 40° C. for at least 1 month. In some embodiments, midostaurin topical formulations contains no more than 2 wt % of total impurity after stored at about 40° C. for at least 2 months. In some embodiments, midostaurin topical formulations contains no more than 2 wt % of total impurity after stored at about 40° C. for at least 3 months. In some embodiments, midostaurin topical formulations contains no more than 2 wt % of total impurity after stored at about 40° C. for at least 6 months.

Refrigerated temperature, also as defined by the USP, is between 2 and 8 degrees Celsius, and is sometimes designated by the nominal value of 5 degrees Celsius. In some embodiments, refrigerated temperatures can be defined as 5±3° C. Refrigerated conditions include temperature and/or relative humidity (RH) in typical refrigeration units (e.g., 5±3° C.). In some instances, a refrigerated condition is about 2° C., about 3° C., about 4° C., about 5° C., about 6° C., about 7° C., or about 8° C. In some instances, a refrigerated condition is about 2° C. to about 8° C.

As used herein, the term “room temperature,” “ambient conditions,” or “ambient temperature” refers to room temperature or “controlled room temperature”. In some embodiments, the room temperatures are about 15° C. to about 25° C. In some embodiments, the ambient conditions comprise temperatures that are about 15° C. to about 25° C. In some embodiments, the room temperatures are 25±5° C. In some embodiments, the controlled room temperatures are about 20° C. to about 25° C. In some embodiments, ambient conditions are about 25±5° C. and 60±5% RH. In some embodiments, ambient conditions are about 25±2° C. and 60±5% RH. In some embodiments, the room temperature is about 25° C. and about 60% RH. In some instances, a room temperature or ambient temperature is at about 20° C. about 21° C., about 22° C., about 23° C., about 24° C., about 25° C., about 26° C., about 27° C., about 28° C., about 29° C., and about 30° C. In other instances, an ambient condition is about 55% RH, about 60% RH. or about 65% RH.

Accelerated conditions for the midostaurin topical pharmaceutical formulations described herein include temperature and/or relative humidity (RH) that are at or above ambient levels or room temperature (e.g., 25+5° C.; 55+10% RH). In some instances, an accelerated condition is at about 25° C., about 30° C., about 35° C., about 40° C., about 45° C., about 50° C., about 55° C., or about 60° C. In other instances, an accelerated condition is above 55% RH, about 65% RH, about 70% RH, about 75% RH or about 80% RH. In further instances, an accelerated condition is about 40° C.±2° C. or 60° C. at ambient humidity. In yet further instances, an accelerated condition is about 40° C.±2° C. at 75+5% RH humidity.

Freeze-Thaw studies can comprise subjecting pharmaceutical compositions through a series of extreme temperature changes, which are called freeze-thaw cycles or freeze thaw cycles. An example of a freeze-thaw cycle is exposing the pharmaceutical compositions to freezing temperatures (for example. about −15° C. to about −25° C.) for 24 hours and then store it at a higher temperature (for example, about 25° C.) for 24 hours. In some embodiments, a freeze-thaw cycle refers to placing a pharmaceutical composition under about 60° C. for about 6 hours, followed by placing the same pharmaceutical composition under about-20° C. for about 24 hours.

### Dosing

Pharmaceutical compositions described herein are administered for the treatment or prevention of diseases. When used to treat or prevent diseases or disorders, pharmaceutical compositions are administered or applied singly, or in combination with other agents. Pharmaceutical compositions may also be administered or applied singly, in combination with other pharmaceutically active agents. Provided herein are methods of treatment and prophylaxis by administration to a subject in need of such treatment of a therapeutically effective amount of a topical pharmaceutical composition of the present disclosure. In some embodiments, the subject is an animal, e.g., a mammal such as a human.

In some embodiments, the pharmaceutical compositions are administered topically. In some embodiments, the pharmaceutical compositions are administered in a topical dosage form. In some embodiments, the pharmaceutical compositions are administered as an ointment, a gel, a cream, a lotion, a solution, an emulsion, a paste, a patch, a wipe, a swab, a pad, or any suitable form. In some embodiments, the pharmaceutical compositions are administered as a gel. In some embodiments, the pharmaceutical compositions are administered as a cream. In some embodiments, the pharmaceutical compositions are administered as an ointment.

In some embodiments, the pharmaceutical composition is formulated for topical administration. In some embodiments, the pharmaceutical composition is formulated for once daily dosing. In some embodiments, the pharmaceutical composition is formulated for twice daily dosing. In some embodiments, the pharmaceutical composition is formulated to be administered 3 times or more per day.

Dosages of midostaurin topical pharmaceutical compositions described can be determined by any suitable method. Maximum tolerated doses (MTD) and maximum response doses (MRD) for midostaurin can be determined via established animal and human experimental protocols as well as in the examples described herein. For example, toxicity and therapeutic efficacy of midostaurin can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Midostaurin dosages exhibiting high therapeutic indices are of interest. The data obtained from cell culture assays and animal studies can be used in formulating a range of dosage for use in human. The dosage of such compounds lies preferably within a range of circulating concentrations that include the EDso with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given midostaurin topical pharmaceutical composition that corresponds to such an amount varies depending upon factors such as the particular midostaurin salt or form, disease condition and its severity, the identity (e.g., weight, sex) of the subject or host in need of treatment, but can nevertheless be determined according to the particular circumstances surrounding the case, including, e.g., the specific agent being administered, the liquid composition type, the condition being treated, and the subject or host being treated. In some instances, the midostaurin topical pharmaceutical compositions can be discontinued if there is a lack of satisfactory response is noted.

In some embodiments, midostaurin topical pharmaceutical compositions described herein are provided in a dose per day from about 0.001 mg to 300 mg, from about 0.01 mg to about 200 mg, from about 0.1 mg to about 20 mg, from about 0.2 mg to about 10 mg of midostaurin. In certain embodiments. the midostaurin topical pharmaceutical compositions described herein are provided in a daily dose of about 0.001 mg to about 0.005 mg, about 0.005 mg to about 0.05 mg, about 0.05 mg to about 0.1 mg. about 0.1 mg to about 0.15 mg, about 0.15 mg to about 0.2 mg, about 0.2 mg to about 0.25 mg, about 0.25 mg to about 0.3 mg, about 0.3 mg to about 0.35 mg, about 0.35 mg to about 0.4 mg, about 0.4 mg to about 0.45 mg, about 0.45 mg to about 0.5 mg, about 0.5 mg to about 0.6 mg, about 0.6 mg to about 0.7 mg, about 0.7 mg to about 0.8 mg, about 0.8 mg to about 0.9 mg, about 0.9 mg to about 1 mg, about 1 mg to about 2 mg, about 2 mg to about 3 mg, about 3 mg to about 4 mg, about 4 mg to about 5 mg, about 5 mg to about 6 mg, about 6 mg to about 7 mg, about 7 mg to about 8 mg, about 8 mg to about 9 mg, about 9 mg to about 10 mg, about 10 mg to about 11 mg, about 11 mg to about 12 mg, about 12 mg to about 15 mg, about 15 mg to about 30 mg, about 30 mg to about 60 mg, about 60 mg to about 90 mg, about 90 mg to about 120 mg, about 120 mg to about 150 mg, about 150 mg to about 200 mg, or about 200 mg to about 300 mg. In certain instances, the midostaurin topical pharmaceutical compositions described herein are provided in a dose per day of about 0.1 mg. In certain instances, the midostaurin topical pharmaceutical compositions described herein are provided in a dose per day of about 0.15 mg. In certain instances, the midostaurin topical pharmaceutical compositions described herein are provided in a dose per day of about 0.2 mg. In certain instances, the midostaurin topical pharmaceutical compositions described herein are provided in a dose per day of about 0.25 mg. In certain instances, the midostaurin topical pharmaceutical compositions described herein are provided in a dose per day of about 0.3 mg. In certain instances, the midostaurin topical pharmaceutical compositions described herein are provided in a dose per day of about 0.35 mg. In certain instances, the midostaurin topical pharmaceutical compositions described herein are provided in a dose per day of about 0.4 mg. In certain instances, the midostaurin topical pharmaceutical compositions described herein are provided in a dose per day of about 0.5 mg. In certain instances, the midostaurin topical pharmaceutical compositions described herein are provided in a dose per day of about 0.6 mg. In certain instances, the midostaurin topical pharmaceutical compositions described herein are provided in a dose per day of about 0.7 mg. In certain instances, the midostaurin topical pharmaceutical compositions described herein are provided in a dose per day of about 0.8 mg. In certain instances, the midostaurin topical pharmaceutical compositions described herein are provided in a dose per day of about 0.9 mg. In certain instances, the midostaurin topical pharmaceutical compositions described herein are provided in a dose per day of about 1 mg. In certain instances, the midostaurin topical pharmaceutical compositions described herein are provided in a dose per day of about 2 mg. In certain instances, the midostaurin topical pharmaceutical compositions described herein are provided in a dose per day of about 3 mg. In certain instances, the midostaurin topical pharmaceutical compositions described herein are provided in a dose per day of about 4 mg. In certain instances, the midostaurin topical pharmaceutical compositions described herein are provided in a dose per day of about 5 mg. In certain instances, the midostaurin topical pharmaceutical compositions described herein are provided in a dose per day of about 6 mg. In certain instances, the midostaurin topical pharmaceutical compositions described herein are provided in a dose per day of about 7 mg. In certain instances, the midostaurin topical pharmaceutical compositions described herein are provided in a dose per day of about 8 mg. In certain instances, the midostaurin topical pharmaceutical compositions described herein are provided in a dose per day of about 9 mg. In certain instances, the midostaurin topical pharmaceutical compositions described herein are provided in a dose per day of about 10 mg. In certain instances, the midostaurin topical pharmaceutical compositions described herein are provided in a dose per day of about 11 mg. In certain instances, the midostaurin topical pharmaceutical compositions described herein are provided in a dose per day of about 12 mg. In certain instances, the midostaurin topical pharmaceutical compositions described herein are provided in a dose per day of about 13 mg. In certain instances, the midostaurin topical pharmaceutical compositions described herein are provided in a dose per day of about 14 mg. In certain instances, the midostaurin topical pharmaceutical compositions described herein are provided in a dose per day of about 15 mg. In certain instances, the midostaurin topical pharmaceutical compositions described herein are provided in a dose per day of about 16 mg. In certain instances, the midostaurin topical pharmaceutical compositions described herein are provided in a dose per day of about 17 mg. In certain instances, the midostaurin topical pharmaceutical compositions described herein are provided in a dose per day of about 18 mg. In certain instances, the midostaurin topical pharmaceutical compositions described herein are provided in a dose per day of about 19 mg. In certain instances, the midostaurin topical pharmaceutical compositions described herein are provided in a dose per day of about 20 mg. In certain instances, the midostaurin topical pharmaceutical compositions described herein are provided in a dose per day of about 25 mg. In certain instances, the midostaurin topical pharmaceutical compositions described herein are provided in a dose per day of about 30 mg. In certain instances, the midostaurin topical pharmaceutical compositions described herein are provided in a dose per day of about 60 mg. In certain instances, the midostaurin topical pharmaceutical compositions described herein are provided in a dose per day of about 90 mg. In certain instances, the midostaurin topical pharmaceutical compositions described herein are provided in a dose per day of about 120 mg. In certain instances, the midostaurin topical pharmaceutical compositions described herein are provided in a dose per day of about 200 mg. In certain instances, the midostaurin topical pharmaceutical compositions described herein are provided in a dose per day of about 300 mg. The dose per day described herein can be given once per day or multiple times per day in the form of sub-doses given b.i.d., t.i.d., q.i.d., or the like where the number of sub-doses equal the dose per day.

In other embodiments, the midostaurin topical pharmaceutical compositions are provided at the maximum tolerated dose (MTD) for midostaurin or a pharmaceutically acceptable salt thereof. In other embodiments, the amount of the midostaurin topical pharmaceutical compositions administered is from about 10% to about 90% of the maximum tolerated dose (MTD), from about 25% to about 75% of the MTD, or about 50% of the MTD. In particular embodiments, the amount of the midostaurin topical pharmaceutical compositions administered is from about 5% to about 10%, about 10% to about 20%. about 20% to about 30%, about 30% to about 40%, about 40% to about 50%, about 50% to about 60%, about 60% to about 70%, about 70) % to about 80%, about 80% to about 90%, about 90% to about 99% or higher, of the MTD for midostaurin or a pharmaceutically acceptable salt thereof.

A topical pharmaceutical composition described herein can be administered at a lower dose than commercially available topical pharmaceutical composition for treating the same indication (e.g., 2% hydroquinone cream) to achieve the same therapeutic effect. In some embodiments, a therapeutically effective amount of the multi-targeting protein kinase inhibitor or a salt thereof (e.g., midostaurin) is a dose that is 80% less than that for hydroquinone. In some embodiments, a therapeutically effective amount of a topical pharmaceutical composition comprising the multi-targeting protein kinase inhibitor or a salt thereof (e.g., midostaurin) is a dose that is 80% less than that for 2% hydroquinone cream. In some embodiments, a therapeutically effective amount of a topical pharmaceutical composition comprising the multi-targeting protein kinase inhibitor or a salt thereof (e.g., midostaurin) is a dose that is 70% less than that for 2% hydroquinone cream. In some embodiments, a therapeutically effective amount of a topical pharmaceutical composition comprising the multi-targeting protein kinase inhibitor or a salt thereof (e.g., midostaurin) is a dose that is 50% less than that for 2% hydroquinone cream. In some embodiments, a topical pharmaceutical composition comprising midostaurin is administered at a dose that is 80% less than that for 2% hydroquinone cream. In some embodiments, a topical pharmaceutical composition comprising midostaurin is administered at a dose that is 60% less than that for 2% hydroquinone cream. In some embodiments, a topical pharmaceutical composition comprising midostaurin is administered at a dose that is 50% less than that for 2% hydroquinone cream. In some embodiments, a topical pharmaceutical composition comprising midostaurin is administered at a dose that is 30% less than that for 2% hydroquinone cream. In some embodiments, a topical pharmaceutical composition comprises at most 1.0% wt midostaurin is administered at a dose that is at most 80% relative to that of 2% hydroquinone cream to achieve the same therapeutic effect. In some embodiments, a topical pharmaceutical composition comprises 0.5% wt midostaurin is administered at a dose that is at most 50% relative to that of 2% hydroquinone cream to achieve the same therapeutic effect. In some embodiments, a topical pharmaceutical composition comprises 0.5% wt midostaurin is administered at a dose that is at most 30% relative to that of 2% hydroquinone cream to achieve the same therapeutic effect.

### Methods of Treatment

In one aspect, disclosed herein is a method of treating a skin-related disease or disorder. comprising topically applying midostaurin or a salt thereof to a skin of a subject, wherein the skin is associated with a skin-related disease or disorder. In one aspect, described herein is a method of treating a disease or condition by topically administering to a subject in need thereof a composition (e.g., cosmetic or pharmaceutical composition) comprising midostaurin or a salt thereof. In one aspect. described herein is a method of treating a disease or condition by topically administering to a subject in need thereof a pharmaceutical composition comprising midostaurin or a pharmaceutically acceptable salt thereof. In some embodiments, the skin-related disease or disorder is skin pigmentation disorder. In some embodiments, the subject has a skin hyperpigmentation condition. In one aspect, midostaurin topical pharmaceutical compositions described herein is used to treat a skin-related disease or disorder. In some embodiments, the method comprises topically applying a pharmaceutical composition described herein to a skin of a subject in need thereof. In some embodiments, the pharmaceutical composition comprises midostaurin or a pharmaceutically acceptable salt thereof. In some embodiments, the skin-related disease or disorder comprises skin hyperpigmentation. In some embodiments, the skin hyperpigmentation is characterized by general darkening of an individual's normal skin color. In some embodiments, the skin hyperpigmentation is characterized by localized darkening of an individual's normal skin color. Skin hyperpigmentation conditions can include, for example, melasma, post-inflammatory hyperpigmentation. posttraumatic pigmentation, discoid lupus erythematous, freckles, plaques gravidarum, nevus of Ota. aging pigmentation and others. In some embodiments, the skin hyperpigmentation condition comprises a sunspot or an age spot. The skin hyperpigmentation can appear on any or all parts of the body. In one aspect, disclosed herein is a method of treating melasma, comprising topically applying midostaurin or a salt thereof to a skin of a subject in need thereof. In some embodiments, the melasma is chloasma. In some embodiments, the treating comprises reducing the size of abnormal skin pigmentation, reducing the intensity of abnormal skin pigmentation, and/or eliminating the abnormal skin pigmentation associated with the melasma. In some embodiments, the method comprises topically applying a pharmaceutical composition, wherein the pharmaceutical composition comprises the midostaurin or a salt thereof. In some embodiments, the salt of midostaurin is a pharmaceutically acceptable salt of midostaurin. In some embodiments, the midostaurin or a salt thereof is administered in a therapeutically effective amount.

In some embodiments, a method described herein comprises applying midostaurin or a salt thereof, or a pharmaceutical or cosmetic composition comprising the midostaurin or the salt thereof to a subject's skin. In some embodiments, the skin is on the subject's face, cheeks, bridge of their nose. forehead, chin, and above their upper lip. In some embodiments, the skin is on the subject's face. In some embodiments, the midostaurin or the salt thereof is administered in a composition, e.g., an ointment, a gel, a cream, a lotion, a solution, an emulsion, a paste, a patch, a wipe, a swab, a pad, and any other form suitable for topical delivery. In some embodiments, the midostaurin or the salt thereof is administered in a transdermal composition. In some embodiments, the midostaurin or the salt thereof is administered in a pharmaceutical composition. In some embodiments, the midostaurin or the salt thereof is administered in a cosmetic composition.

In one aspect, disclosed herein is a method of reducing skin pigmentation, comprising topically applying midostaurin or a salt thereof to a pigmented skin of a subject. In one aspect, the midostaurin topical pharmaceutical compositions described herein is used to reduce skin pigmentation. In some embodiments, the method comprises topically applying a pharmaceutical composition described herein to a skin of a subject in need thereof. In some embodiments, the pharmaceutical composition comprises midostaurin or a pharmaceutically acceptable salt thereof. In some embodiments, the subject has skin-related disorders or conditions. In some embodiments, the skin-related disorders or conditions comprise skin pigmentation disorders. In some embodiments, the skin pigmentation disorders comprise skin hyperpigmentation condition. In some embodiments, the skin hyperpigmentation condition comprises melasma, post-inflammatory hyperpigmentation, posttraumatic pigmentation, discoid lupus erythematous, freckles, plaques gravidarum, nevus of Ota, aging pigmentation and others. In some embodiments, the midostaurin or a salt thereof, or a pharmaceutical composition comprising the midostaurin or the salt thereof is applied to a pigmented skin. The pigment skin can be a pigmented birthmark, macular stain, hemangiomas, or port wine stain. The pigment skin can be a sunspot. The pigment skin can be a dark spot. The pigment skin can be an age spot. The pigment skin can be associated with a skin-related disease or disorder. In some embodiments, the skin-related disease or disorder is melasma or post-inflammatory hyperpigmentation.

In one aspect, disclosed herein is a method of reducing tyrosinase activity, comprising topically applying midostaurin or a pharmaceutically acceptable salt thereof to a skin of a subject in need thereof. In another aspect, the midostaurin topical pharmaceutical compositions described herein is used to reduce tyrosinase activity. In some embodiments, the method comprises topically applying a pharmaceutical composition described herein to a skin of a subject in need thereof. In some embodiments, the topical pharmaceutical composition comprises midostaurin or a pharmaceutically acceptable salt thereof. In some embodiments, the subject has skin-related disorders or conditions. In some embodiments, the skin-related disorders or conditions comprise skin pigmentation disorders. In some embodiments, the skin pigmentation disorders comprise skin hyperpigmentation condition. In some embodiments, the skin hyperpigmentation condition comprises melasma, post-inflammatory hyperpigmentation, posttraumatic pigmentation, discoid lupus erythematous, freckles, plaques gravidarum, nevus of Ota, aging pigmentation and others.

Melasma is a common skin problem that leads to skin pigmentation problems such as brown to gray-brown patches, usually on the face, cheeks, bridge of their nose, forehead, chin, and above their upper lip. Melasma is believed to be caused by or exacerbated by birth control pills, pregnancy, and hormone therapy, stress, thyroid disease, sunlight exposure, inflammation, familial predisposition, or free radicals. In some embodiments, the melasma is caused by or exacerbated by one or more of; birth control pills, hormone therapy, sunlight exposure, or familial predisposition. Sun exposure is believed to cause melasma because ultraviolet rays affect the cells that control pigment (melanocytes). UV radiation can cause lipids peroxidation in cellular membranes, resulting in free radicals which could stimulate melanocytes to produce excess melanin.

In one aspect, disclosed herein is a method of reducing skin melanin content in the skin of a subject desirous of a lighter skin tone, comprising topically applying midostaurin or a salt thereof to a pigmented skin of a subject. In one aspect, disclosed herein is a method of reducing skin pigmentation of a subject desirous of a lighter skin tone, comprising topically applying midostaurin or a salt thereof to a pigmented skin of a subject. In some embodiments, the midostaurin or a salt thereof is formulated in a cosmetic composition.

In some embodiments, the subject is an adult. In some embodiments, the subject is a female. In some embodiments, the subject is a male. In some embodiments, the subject is a child. In some embodiments, the subject is at least one year old. In some embodiments, the subject is less than one year old. In some embodiments, the subject is 1 to 12 years old. In some embodiments, the subject is 1 to 18 years old. In some embodiments, the subject is 12 to 18 years old. In some embodiments, the subject is at least 18 years old. In some embodiments, the subject is at least 24 years old. In some embodiments, the subject is 1 to 90 years old. In some embodiments, the subject has received or is receiving hormone therapy. In some embodiments, the subject has received or is receiving birth control pills. In some embodiments, the subject has familial predisposition of having hyperpigmentation or melasma.

In some embodiments, pharmaceutical compositions described herein can be used in combination therapy with at least one other therapeutic agent. The pharmaceutical composition and the therapeutic agent can act additively or, more preferably, synergistically. In some embodiments, the pharmaceutical composition is administered concurrently with the administration of another therapeutic agent. In some embodiments, a pharmaceutical composition is administered prior or subsequent to administration of another therapeutic agent.

In one aspect, midostaurin or a salt thereof reduce melanin amount in an area of skin. In some embodiments, the midostaurin or a salt thereof is formulated as a topical pharmaceutical composition described herein. In some embodiments, the topical pharmaceutical composition comprises midostaurin or a pharmaceutically acceptable salt thereof. In some embodiments, the midostaurin or a salt thereof reduces melanin amount in an area of skin by at least 10%, 15%, 20%, 25%, 30%, 35%, 40%, 45%, 50%, 60%, 70%, 80%, 90%, 95%, or 99% compared to a similar area of skin in the same subject without any treatment. In some embodiments, the midostaurin or a salt thereof reduces melanin amount in an area of skin by at least 25% compared to a similar area of skin in the same subject without any treatment. In some embodiments, the midostaurin or a salt thereof reduces melanin amount in an area of skin by at least 50% compared to a similar area of skin in the same subject without any treatment. In some embodiments, the midostaurin or a salt thereof reduces melanin amount in an area of skin by at least 75% compared to a similar area of skin in the same subject without any treatment. In some embodiments, the midostaurin topical pharmaceutical composition described herein reduces melanin amount in an area of skin by at least 10%, 15%, 20%, 25%, 30%, 35%, 40%, 45%, 50%, 60%, 70%, 80%, 90%, 95%, or 99% compared to a similar area of skin in the same subject without any treatment. In some embodiments, the midostaurin topical pharmaceutical composition described herein reduces melanin amount in an area of skin by at least 10% compared to a similar area of skin in the same subject without any treatment. In some embodiments, the midostaurin topical pharmaceutical composition described herein reduces melanin amount in an area of skin by at least 15% compared to a similar area of skin in the same subject without any treatment. In some embodiments, the midostaurin topical pharmaceutical composition described herein reduces melanin amount in an area of skin by at least 20% compared to a similar area of skin in the same subject without any treatment. In some embodiments, the midostaurin topical pharmaceutical composition described herein reduces melanin amount in an area of skin by at least 25% compared to a similar area of skin in the same subject without any treatment. In some embodiments, the midostaurin topical pharmaceutical composition described herein reduces melanin amount in an area of skin by at least 30% compared to a similar area of skin in the same subject without any treatment. In some embodiments, the midostaurin topical pharmaceutical composition described herein reduces melanin amount in an area of skin by at least 35% compared to a similar area of skin in the same subject without any treatment. In some embodiments, the midostaurin topical pharmaceutical composition described herein reduces melanin amount in an area of skin by at least 40% compared to a similar area of skin in the same subject without any treatment. In some embodiments, the midostaurin topical pharmaceutical composition described herein reduces melanin amount in an area of skin by at least 45% compared to a similar area of skin in the same subject without any treatment. In some embodiments, the midostaurin topical pharmaceutical composition described herein reduces melanin amount in an area of skin by at least 50% compared to a similar area of skin in the same subject without any treatment. In some embodiments, the midostaurin topical pharmaceutical composition described herein reduces melanin amount in an area of skin by at least 60% compared to a similar area of skin in the same subject without any treatment. In some embodiments, the midostaurin topical pharmaceutical composition described herein reduces melanin amount in an area of skin by at least 70% compared to a similar area of skin in the same subject without any treatment. In some embodiments, the midostaurin topical pharmaceutical composition described herein reduces melanin amount in an area of skin by at least 80% compared to a similar area of skin in the same subject without any treatment. In some embodiments, the midostaurin topical pharmaceutical composition described herein reduces melanin amount in an area of skin by at least 90% compared to a similar area of skin in the same subject without any treatment. In some embodiments, the midostaurin topical pharmaceutical composition described herein reduces melanin amount in an area of skin by at least 95% compared to a similar area of skin in the same subject without any treatment. In some embodiments, the midostaurin topical pharmaceutical composition described herein reduces melanin amount in an area of skin by at least 99% compared to a similar area of skin in the same subject without any treatment. In some embodiments, the melanin amount is measured by a melanin distribution score, such as the method described in Example G. In some embodiments, the melanin amount is measured by a melanin distribution score using guinea pig as a model.

In some embodiments, midostaurin or a salt thereof reduces melanin amount in an area of skin by at least 10%, 15%, 20%, 25%, 30%, 35%, 40%, 45%, 50%, 60%, 70%, 80%, 90%, 95%, or 99% compared to a similar area of skin in the same subject receiving a treatment of a corresponding composition comprising hydroquinone. In some embodiments, midostaurin or a salt thereof reduces melanin amount in an area of skin by at least 25% compared to a similar area of skin in the same subject receiving a treatment of a corresponding composition comprising hydroquinone. In some embodiments, midostaurin or a salt thereof reduces melanin amount in an area of skin by at least 50% compared to a similar area of skin in the same subject receiving a treatment of a corresponding composition comprising hydroquinone. In some embodiments, midostaurin or a salt thereof reduces melanin amount in an area of skin by at least 75% compared to a similar area of skin in the same subject receiving a treatment of a corresponding composition comprising hydroquinone. In some embodiments, a midostaurin topical pharmaceutical composition described herein reduces melanin amount in an area of skin by at least 10%, 20%, 30%, 40%, 50%, 60%, 70%, 80%, 90%, 95%, or 99% compared to a similar area of skin in the same subject receiving a treatment of a corresponding composition comprising hydroquinone. In some embodiments, the midostaurin topical pharmaceutical composition described herein reduces melanin amount in an area of skin by at least 10% compared to a similar area of skin in the same subject receiving a treatment of a corresponding composition comprising hydroquinone. In some embodiments, the midostaurin topical pharmaceutical composition described herein reduces melanin amount in an area of skin by at least 20% compared to a similar area of skin in the same subject receiving a treatment of a corresponding composition comprising hydroquinone. In some embodiments, the midostaurin topical pharmaceutical composition described herein reduces melanin amount in an area of skin by at least 30% compared to a similar area of skin in the same subject receiving a treatment of a corresponding composition comprising hydroquinone. In some embodiments, the midostaurin topical pharmaceutical composition described herein reduces melanin amount in an area of skin by at least 40% compared to a similar area of skin in the same subject receiving a treatment of a corresponding composition comprising hydroquinone. In some embodiments, the midostaurin topical pharmaceutical composition described herein reduces melanin amount in an area of skin by at least 50% compared to a similar area of skin in the same subject receiving a treatment of a corresponding composition comprising hydroquinone. In some embodiments, the midostaurin topical pharmaceutical composition described herein reduces melanin amount in an area of skin by at least 60% compared to a similar area of skin in the same subject receiving a treatment of a corresponding composition comprising hydroquinone. In some embodiments, the midostaurin topical pharmaceutical composition described herein reduces melanin amount in an area of skin by at least 70% compared to a similar area of skin in the same subject receiving a treatment of a corresponding composition comprising hydroquinone. In some embodiments, the midostaurin topical pharmaceutical composition described herein reduces melanin amount in an area of skin by at least 80% compared to a similar area of skin in the same subject receiving a treatment of a corresponding composition comprising hydroquinone. In some embodiments, the midostaurin topical pharmaceutical composition described herein reduces melanin amount in an area of skin by at least 90% compared to a similar area of skin in the same subject receiving a treatment of a corresponding composition comprising hydroquinone. In some embodiments, the midostaurin topical pharmaceutical composition described herein reduces melanin amount in an area of skin by at least 95% compared to a similar area of skin in the same subject receiving a treatment of a corresponding composition comprising hydroquinone. In some embodiments, the midostaurin topical pharmaceutical composition described herein reduces melanin amount in an area of skin by at least 99% compared to a similar area of skin in the same subject receiving a treatment of a corresponding composition comprising hydroquinone. In some embodiments, the melanin amount is measured by a melanin distribution score, such as the method described in Example G. In some embodiments, the melanin amount is measured by a melanin distribution score using guinea pig as a model.

In some embodiments, a topical pharmaceutical composition comprising midostaurin or a salt thereof reduces melanin amount in an area of skin. In some embodiments, a reduction of melanin amount in an area of skin treated with the topical pharmaceutical composition is at least 100%, 110%, 120%, 130%, or 150% relative to that of a similar area of skin in the same subject treated with a reference topical pharmaceutical composition comprising hydroquinone, wherein the topical pharmaceutical composition is administered at a dose that is lower than a dose of the reference topical pharmaceutical composition. In some cases, the reference topical pharmaceutical composition comprising hydroquinone is 2% hydroquinone cream that is commercially available. In some cases, the topical pharmaceutical composition comprises at most 1.5% wt midostaurin. In some cases, a reduction of melanin amount in an area of skin treated with the topical pharmaceutical composition is at least 130% relative to that of a similar area of skin in the same subject treated with 2% hydroquinone cream, and wherein the topical pharmaceutical composition comprises 1.5% wt midostaurin. In some cases, a reduction of melanin amount in an area of skin treated with the topical pharmaceutical composition is at least 90% relative to that of a similar area of skin in the same subject treated with 2% hydroquinone cream, and wherein the topical pharmaceutical composition comprises 0.25% wt midostaurin. In some cases, a reduction of melanin amount in an area of skin treated with the topical pharmaceutical composition is at least 50% relative to that of a similar area of skin in the same subject treated with 2% hydroquinone cream, and wherein the topical pharmaceutical composition comprises 0.05% wt midostaurin. In some cases, a reduction of melanin amount in an area of skin treated with the topical pharmaceutical composition is at least 50% relative to that of a similar area of skin in the same subject treated with 2% hydroquinone cream, and wherein the topical pharmaceutical composition comprises 0.01% wt midostaurin. In some embodiments, the melanin amount is measured by a melanin distribution score, such as the method described in Example G. In some embodiments, the melanin amount is measured by a melanin distribution score using guinea pig as a model.

In some embodiments, midostaurin or a salt thereof reduces melanin amount in an area of skin by at least 0.5, 1.0, 1.5, 2.0, 2.5 or 3.0 according to Melanin Distribution Score compared to a similar area of skin in the same subject without the treatment. In some embodiments, midostaurin or a salt thereof reduces melanin amount in an area of skin by at least 1.0 according to Melanin Distribution Score compared to a similar area of skin in the same subject without the treatment. In some embodiments, midostaurin or a salt thereof reduces melanin amount in an area of skin by at least 2.0 according to Melanin Distribution Score compared to a similar area of skin in the same subject without the treatment. In some embodiments, midostaurin or a salt thereof reduces melanin amount in an area of skin by at least 1, 2, 3, or 4 according to Fontana-Masson staining scoring standard compared to a similar area of skin in the same subject without the treatment. In some embodiments, midostaurin or a salt thereof reduces melanin amount in an area of skin by at least 1 according to Fontana-Masson staining scoring standard compared to a similar area of skin in the same subject without the treatment. In some embodiments, midostaurin or a salt thereof reduces melanin amount in an area of skin by at least 2 according to Fontana-Masson staining scoring standard compared to a similar area of skin in the same subject without the treatment. In some embodiments, the midostaurin or a salt thereof is formulated as a topical pharmaceutical composition described herein. In some embodiments, the topical pharmaceutical composition comprises midostaurin or a pharmaceutically acceptable salt thereof.

In one aspect, midostaurin or a salt thereof reduces tyrosinase activity in an area of skin. In some embodiments, the midostaurin or a salt thereof is formulated as a topical pharmaceutical composition described herein. In some embodiments, the topical pharmaceutical composition comprises midostaurin or a pharmaceutically acceptable salt thereof. In some embodiments, midostaurin or a salt thereof reduces tyrosinase activity in an area of skin by at least 10%, 15%, 20%, 25%, 30%, 35%, 40%, 45%, 50%, 60%, 70%, 80%, 90%, 95% or 99% compared to a similar area of skin in the same subject without any treatment. In some embodiments, midostaurin or a salt thereof reduces tyrosinase activity in an area of skin by at least 25% compared to a similar area of skin in the same subject without any treatment. In some embodiments, midostaurin or a salt thereof reduces tyrosinase activity in an area of skin by at least 50% compared to a similar area of skin in the same subject without any treatment. In some embodiments, midostaurin or a salt thereof reduces tyrosinase activity in an area of skin by at least 75% compared to a similar area of skin in the same subject without any treatment. In some embodiments, a midostaurin topical pharmaceutical composition described herein reduces tyrosinase activity in an area of skin by at least 10%, 15%, 20%, 25%, 30%, 35%, 40%, 45%, 50%, 60%, 70%, 80%, 90%, 95% or 99% compared to a similar area of skin in the same subject without any treatment. In some embodiments, the midostaurin topical pharmaceutical composition described herein reduces tyrosinase activity in an area of skin by at least 10% compared to a similar area of skin in the same subject without any treatment. In some embodiments, the midostaurin topical pharmaceutical composition described herein reduces tyrosinase activity in an area of skin by at least 15% compared to a similar area of skin in the same subject without any treatment. In some embodiments, the midostaurin topical pharmaceutical composition described herein reduces tyrosinase activity in an area of skin by at least 20% compared to a similar area of skin in the same subject without any treatment. In some embodiments, the midostaurin topical pharmaceutical composition described herein reduces tyrosinase activity in an area of skin by at least 25% compared to a similar area of skin in the same subject without any treatment. In some embodiments, the midostaurin topical pharmaceutical composition described herein reduces tyrosinase activity in an area of skin by at least 30% compared to a similar area of skin in the same subject without any treatment. In some embodiments, the midostaurin topical pharmaceutical composition described herein reduces tyrosinase activity in an area of skin by at least 35% compared to a similar area of skin in the same subject without any treatment. In some embodiments, the midostaurin topical pharmaceutical composition described herein reduces tyrosinase activity in an area of skin by at least 40% compared to a similar area of skin in the same subject without any treatment. In some embodiments, the midostaurin topical pharmaceutical composition described herein reduces tyrosinase activity in an area of skin by at least 45% compared to a similar area of skin in the same subject without any treatment. In some embodiments, the midostaurin topical pharmaceutical composition described herein reduces tyrosinase activity in an area of skin by at least 50% compared to a similar area of skin in the same subject without any treatment. In some embodiments, the midostaurin topical pharmaceutical composition described herein reduces tyrosinase activity in an area of skin by at least 60% compared to a similar area of skin in the same subject without any treatment. In some embodiments, the midostaurin topical pharmaceutical composition described herein reduces tyrosinase activity in an area of skin by at least 70% compared to a similar area of skin in the same subject without any treatment. In some embodiments, the midostaurin topical pharmaceutical composition described herein reduces tyrosinase activity in an area of skin by at least 80% compared to a similar area of skin in the same subject without any treatment. In some embodiments, the midostaurin topical pharmaceutical composition described herein reduces tyrosinase activity in an area of skin by at least 90% compared to a similar area of skin in the same subject without any treatment. In some embodiments, the midostaurin topical pharmaceutical composition described herein reduces tyrosinase activity in an area of skin by at least 95% compared to a similar area of skin in the same subject without any treatment. In some embodiments, the midostaurin topical pharmaceutical composition described herein reduces tyrosinase activity in an area of skin by at least 99% compared to a similar area of skin in the same subject without any treatment. In some embodiments, the tyrosinase activity is measured per gram of tissue, such as using the method described in Example G. In some embodiments, the tyrosinase activity is measured per gram of tissue using guinea pig as a model.

In some embodiments, midostaurin or a salt thereof reduces tyrosinase activity in an area of skin by at least 10%, 20%, 30%, 40%, 50%, 60%, 70%, 80%, 90%, 95%, or 99% compared to a similar area of skin in the same subject receiving a treatment of a corresponding composition comprising hydroquinone. In some embodiments, the midostaurin or a salt thereof is formulated as a topical pharmaceutical composition described herein. In some embodiments, the topical pharmaceutical composition comprises midostaurin or a pharmaceutically acceptable salt thereof. In some embodiments, midostaurin or a salt thereof reduces tyrosinase activity in an area of skin by at least 25% compared to a similar area of skin in the same subject receiving a treatment of a corresponding composition comprising hydroquinone. In some embodiments, midostaurin or a salt thereof reduces tyrosinase activity in an area of skin by at least 55% compared to a similar area of skin in the same subject receiving a treatment of a corresponding composition comprising hydroquinone. In some embodiments, midostaurin or a salt thereof reduces tyrosinase activity in an area of skin by at least 75% compared to a similar area of skin in the same subject receiving a treatment of a corresponding composition comprising hydroquinone. In some embodiments, the midostaurin topical pharmaceutical composition described herein reduces tyrosinase activity in an area of skin by at least 10% compared to a similar area of skin in the same subject receiving a treatment of a corresponding composition comprising hydroquinone. In some embodiments, the midostaurin topical pharmaceutical composition described herein reduces tyrosinase activity in an area of skin by at least 20% compared to a similar area of skin in the same subject receiving a treatment of a corresponding composition comprising hydroquinone. In some embodiments, the midostaurin topical pharmaceutical composition described herein reduces tyrosinase activity in an area of skin by at least 30% compared to a similar area of skin in the same subject receiving a treatment of a corresponding composition comprising hydroquinone. In some embodiments, the midostaurin topical pharmaceutical composition described herein reduces tyrosinase activity in an area of skin by at least 40% compared to a similar area of skin in the same subject receiving a treatment of a corresponding composition comprising hydroquinone. In some embodiments, the midostaurin topical pharmaceutical composition described herein reduces tyrosinase activity in an area of skin by at least 50% compared to a similar area of skin in the same subject receiving a treatment of a corresponding composition comprising hydroquinone. In some embodiments, the midostaurin topical pharmaceutical composition described herein reduces tyrosinase activity in an area of skin by at least 60% compared to a similar area of skin in the same subject receiving a treatment of a corresponding composition comprising hydroquinone. In some embodiments, the midostaurin topical pharmaceutical composition described herein reduces tyrosinase activity in an area of skin by at least 70% compared to a similar area of skin in the same subject receiving a treatment of a corresponding composition comprising hydroquinone. In some embodiments, the midostaurin topical pharmaceutical composition described herein reduces tyrosinase activity in an area of skin by at least 80% compared to a similar area of skin in the same subject receiving a treatment of a corresponding composition comprising hydroquinone. In some embodiments, the midostaurin topical pharmaceutical composition described herein reduces tyrosinase activity in an area of skin by at least 90% compared to a similar area of skin in the same subject receiving a treatment of a corresponding composition comprising hydroquinone. In some embodiments, the midostaurin topical pharmaceutical composition described herein reduces tyrosinase activity in an area of skin by at least 95% compared to a similar area of skin in the same subject receiving a treatment of a corresponding composition comprising hydroquinone. In some embodiments, the midostaurin topical pharmaceutical composition described herein reduces tyrosinase activity in an area of skin by at least 99% compared to a similar area of skin in the same subject receiving a treatment of a corresponding composition comprising hydroquinone. In some embodiments, the tyrosinase activity is measured per gram of tissue, such as using the method described in Example G. In some embodiments, the tyrosinase activity is measured per gram of tissue using guinea pig as a model.

In some embodiments, midostaurin or a topical pharmaceutical composition comprising midostaurin or a salt thereof reduces tyrosinase activity in an area of skin. In some embodiments, a reduction of tyrosinase activity in an area of skin treated with the topical pharmaceutical composition is at least 100%, 110%, 120%, 130%, 150% relative to that of a similar area of skin in the same subject treated with a reference topical pharmaceutical composition comprising hydroquinone, wherein the topical pharmaceutical composition is administered at a dose that is lower than a dose of the reference topical pharmaceutical composition. In some cases, the reference topical pharmaceutical composition comprising hydroquinone is 2% hydroquinone cream that is commercially available. In some cases, the topical pharmaceutical composition comprises at most 1.5% wt midostaurin. In some cases, a reduction of tyrosinase activity in an area of skin treated with the topical pharmaceutical composition is at least 120% relative to that of a similar area of skin in the same subject treated with 2% hydroquinone cream, and wherein the topical pharmaceutical composition comprises 1.5% wt midostaurin. In some cases, a reduction of tyrosinase activity in an area of skin treated with the topical pharmaceutical composition is at least 90% relative to that of a similar area of skin in the same subject treated with 2% hydroquinone cream, and wherein the topical pharmaceutical composition comprises 0.25% wt midostaurin. In some cases, a reduction of tyrosinase activity in an area of skin treated with the topical pharmaceutical composition is at least 50% relative to that of a similar area of skin in the same subject treated with 2% hydroquinone cream, and wherein the topical pharmaceutical composition comprises 0.05% wt midostaurin. In some cases, a reduction of tyrosinase activity in an area of skin treated with the topical pharmaceutical composition is at least 50% relative to that of a similar area of skin in the same subject treated with 2% hydroquinone cream, and wherein the topical pharmaceutical composition comprises 0.01% wt midostaurin. In some embodiments, the tyrosinase activity is measured per gram of tissue, such as using the method described in Example G. In some embodiments, the tyrosinase activity is measured per gram of tissue using guinea pig as a model.

In some embodiments, midostaurin or a topical pharmaceutical composition comprising midostaurin or a salt thereof causes low skin irritation. In some embodiments, a skin irritation level in an area of skin treated with the topical pharmaceutical composition is at most mild, and wherein the topical pharmaceutical composition comprises at most 1.0% wt midostaurin. In some embodiments, a skin irritation level in an area of skin treated with the topical pharmaceutical composition is mild, and wherein the topical pharmaceutical composition comprises 1.0% wt midostaurin. In some embodiments, a skin irritation level in an area of skin treated with the topical pharmaceutical composition is mild, and wherein the topical pharmaceutical composition comprises 0.5% wt midostaurin. In some embodiments, a skin irritation level in an area of skin treated with the topical pharmaceutical composition is non-irritating, and wherein the topical pharmaceutical composition comprises at most 0.5% wt midostaurin. In some embodiments, a skin irritation score in an area of skin treated with the topical pharmaceutical composition is at most 2.0, and wherein the topical pharmaceutical composition comprises 1.0% wt midostaurin. In some embodiments, a skin irritation score in an area of skin treated with the topical pharmaceutical composition is at most 1.5, and wherein the topical pharmaceutical composition comprises 1.0% wt midostaurin. In some embodiments, a skin irritation score in an area of skin treated with the topical pharmaceutical composition is at most 1.2, and wherein the topical pharmaceutical composition comprises 1.0% wt midostaurin. In some embodiments, a skin irritation score in an area of skin treated with the topical pharmaceutical composition is at most 1.0, and wherein the topical pharmaceutical composition comprises 0.5% wt midostaurin. In some embodiments, the skin irritation score is evaluated by a suitable standard set forth by a regulatory agency, such as the method described in Example H. In some embodiments, skin irritation score and irritation level are measured using rabbit as a model.

In some embodiments, a topical pharmaceutical composition comprising midostaurin or a salt thereof causes lower skin irritation relative to a commercially available reference topical pharmaceutical composition comprising an active ingredient that is not midostaurin (e.g., 2% hydroquinone cream). In some embodiments, a method described herein does not create any skin irritation. In some embodiments, a method described herein does not create any mild skin irritation. In some embodiments, a method described herein does not create any moderate skin irritation. In some embodiments, a method described herein does not create any moderate or severe skin irritation. In some cases, a skin irritation score in an area of skin treated with the topical pharmaceutical composition is at most about 20%, 30%, 40%, 50%, 60%, or 70% relative to that of a similar area of skin in the same subject treated with a reference topical pharmaceutical composition comprising hydroquinone, wherein the topical pharmaceutical composition is administered at a dose that is lower than a dose of the reference topical pharmaceutical composition. In some cases, the reference topical pharmaceutical composition comprising hydroquinone is 2% hydroquinone cream that is commercially available. In some cases, a skin irritation score in an area of skin treated with the topical pharmaceutical composition is at most about 30% relative to that of a similar area of skin in the same subject treated with 2% hydroquinone cream, wherein the topical pharmaceutical composition comprises 1.0% wt midostaurin. In some cases, a skin irritation score in an area of skin treated with the topical pharmaceutical composition is at most about 30% relative to that of a similar area of skin in the same subject treated with 2% hydroquinone cream, wherein the topical pharmaceutical composition comprises 0.5% wt midostaurin. In some embodiments, the skin irritation score is evaluated by a suitable standard set forth by a regulatory agency, such as the method described in Example H. In some embodiments, skin irritation score and irritation level are measured using rabbit as a model. In some embodiments, midostaurin or a topical pharmaceutical composition comprising midostaurin or a salt thereof does not create phototoxicity. In some embodiments, a method described herein does not create phototoxicity in the subject. In some embodiments, midostaurin or a topical pharmaceutical composition comprising midostaurin or a salt thereof does not lead to systemic exposure. In some embodiments, a method described herein does not create systemic exposure of the multi-targeting protein kinase inhibitor (such as midostaurin).

### Kits

For the midostaurin topical pharmaceutical compositions described herein, kits are also described. Such kits can comprise a carrier, package, or container that is compartmentalized to receive one or more containers such as bottles, vials, tubes, and the like, each of the container(s) comprising one of the separate elements to be used in a method described herein including a midostaurin topical pharmaceutical composition, such as an ointment, a cream, or a gel. Suitable containers include, for example, bottles, tubes, vials, and test tubes. The containers can be formed from a variety of materials such as glass or plastic. The containers can further comprise a light protection mechanism. The containers can have different sizes, such as about 12 oz, about 10 oz, about 8 oz, about 4 oz, about 3 oz, about 2 oz, about 1 oz. The containers can have seal, such as induction seal. In some embodiments, the kit comprises a package enclosing the midostaurin topical pharmaceutical composition described herein. In some embodiments, the package is a tube.

A kit can comprise one or more additional containers, each with one or more of various materials (such as devices) desirable from a commercial and user standpoint for a midostaurin topical pharmaceutical composition described herein. Non-limiting examples of such materials include, but not limited to carrier, package, container, vial and/or tube, labels listing contents and/or instructions for use. and package inserts with instructions for use associated with a midostaurin topical pharmaceutical composition. A set of instructions can also be included. In some embodiments, the kit comprises instructions for use of the topical pharmaceutical compositions described herein.

A label can be on or associated with the container, such as a tube. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

### Methods of Manufacture

Preparation of the midostaurin topical pharmaceutical composition described herein includes any known pharmaceutical method.

In one embodiment, the midostaurin topical pharmaceutical composition described herein is prepared by mixing midostaurin or a pharmaceutically acceptable salt thereof with a carrier vehicle (e.g., purified water) and an excipient (e.g., a viscosity modifier, a pH stabilizer, a surfactant, a penetration enhancer, an antioxidant, or any combination thereof) thereby forming a gel. In some embodiments, midostaurin or a pharmaceutically acceptable salt thereof, the carrier vehicle, and the excipient can be combined in any order of addition.

In one embodiment, the midostaurin topical pharmaceutical composition described herein is prepared by mixing midostaurin or a pharmaceutically acceptable salt thereof with a carrier vehicle and an excipient (e.g., a surfactant, a penetration enhancer, an antioxidant, or any combination thereof) thereby forming an ointment. In some embodiments, midostaurin or a pharmaceutically acceptable salt thereof, the carrier vehicle, and the excipient can be combined in any order of addition.

In one embodiment, the midostaurin topical pharmaceutical composition described herein is prepared by mixing midostaurin or a pharmaceutically acceptable salt thereof with a carrier vehicle and an excipient (e.g., a surfactant, a penetration enhancer, a moisturizer, a viscosity modifier, an antioxidant, or any combination thereof) thereby forming a cream. In some embodiments, midostaurin or a pharmaceutically acceptable salt thereof, the carrier vehicle, and the excipient can be combined in any order of addition. In some embodiments, the preparation comprises preparing an oil phase and an aqueous phase. In some embodiments, the oil phase is prepared by mixing midostaurin or a pharmaceutically acceptable salt thereof with a carrier vehicle and an excipient (e.g., a surfactant, optionally a penetration enhancer, optionally a viscosity modifier, optionally a moisturizer) and heating to above 80° C. In some embodiments, the aqueous phase is prepared by weighing water and optionally an excipient (e.g., an antioxidant) and heating to above 80° C. In some embodiments, the preparation comprises gradually adding the oil phase to the aqueous phase and stirring into cream.

### Definitions

Unless specifically stated or obvious from context, as used herein, the term “about” in reference to a number or range of numbers is understood to mean the stated number and numbers +/−10% thereof. or 10% below the lower listed limit and 10% above the higher listed limit for the values listed for a range.

The singular forms “a,” “an,” and, “the” include plural referents unless the context clearly dictates otherwise. Thus, for example, reference to “the surfactant” includes reference to one or more specific surfactants, reference to “an antioxidant” includes reference to one or more of such additives.

The use of the term “or” in the claims is used to mean “and/or” unless explicitly indicated to refer to alternatives only or the alternatives are mutually exclusive, although the disclosure supports a definition that refers to only alternatives and to “and/or.” The terms “comprise.” “have” and “include” are open-ended linking verbs. Any forms or tenses of one or more of these verbs, such as “comprises.” “comprising.” “has.” “having.” “includes” and “including.” are also open-ended. For example, any method that “comprises.” “has” or “includes” one or more steps is not limited to possessing only those one or more steps and covers other unlisted steps.

“Optional” or “optionally” may be taken to mean that the subsequently described structure, event or circumstance may or may not occur, and that the description includes instances where the events occurs and instances where it does not.

As used herein, the term “therapeutic” means an agent utilized to treat, combat, ameliorate, prevent or improve an unwanted condition or disease of a patient. In some embodiments, a therapeutic agent such as midostaurin is directed to the treatment and/or the amelioration of, reversal of, or stabilization of the symptoms of adrenocortical insufficiency described herein.

The term “subject” as used herein refers to a mammal (e.g., a human, mouse, rat, guinea pig, dog, cat, horse, cow, pig, or non-human primate, such as a monkey, chimpanzee or baboon).

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering”, when used in conjunction with a midostaurin formulation, can include, but is not limited to, providing a midostaurin formulation into or onto the target tissue; providing a midostaurin formulation systemically to a patient by, e.g., oral administration whereby the therapeutic reaches the target tissue or cells. “Administering” a formulation may be accomplished by injection, topical administration, and oral administration or by other methods alone or in combination with other known techniques.

In some embodiments, an error-band is included. The term “total error band” is used herein to specify all sources of including sampling and sample preparation calculated at a 95% confidence level. An example is; D50 100 μm with a total error band of +/−5% on size. Other statistics are sometimes used to describe a particle size distribution. The most common calculations are standard deviation and variance. The standard deviation (St Dev.). The standard deviation specification defines the diameter where approximately 68.27% of the total population lies within +/−1 St Dev, and 95.45% lies within +/−2 St Dev.

“Effective amount,” and “sufficient amount” may be used interchangeably and refer to an amount of a substance that is sufficient to achieve an intended purpose or objective.

A “therapeutically effective amount” when used in connection with a pharmaceutical composition described herein is an amount of one or more pharmaceutically active agent(s) sufficient to produce a therapeutic result in a subject in need thereof.

“Therapeutically equivalent” when used in connection with a pharmaceutical composition described herein refers to an amount or quantity of a pharmaceutically acceptable salt or ester of a pharmaceutically active agent that is equivalent to the therapeutically effective amount of the free base or alcohol of the pharmaceutically active agent.

The terms “treat,” “treated,” “treatment,” or “treating” as used herein refers to therapeutic treatment, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition. disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. For example, as used herein, “treating” a hyperpigmented skin includes reducing the size of abnormal skin pigmentation, reducing the intensity of abnormal skin pigmentation, eliminating the abnormal skin pigmentation associated with the melasma, etc.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example A

### Solubility of Midostaurin in Different Solvents and Solubilizer/Water Systems

This example illustrates the process of selecting suitable solvents for a topical formulation for midostaurin or a pharmaceutically acceptable salt thereof, according to some embodiments of the present disclosure.

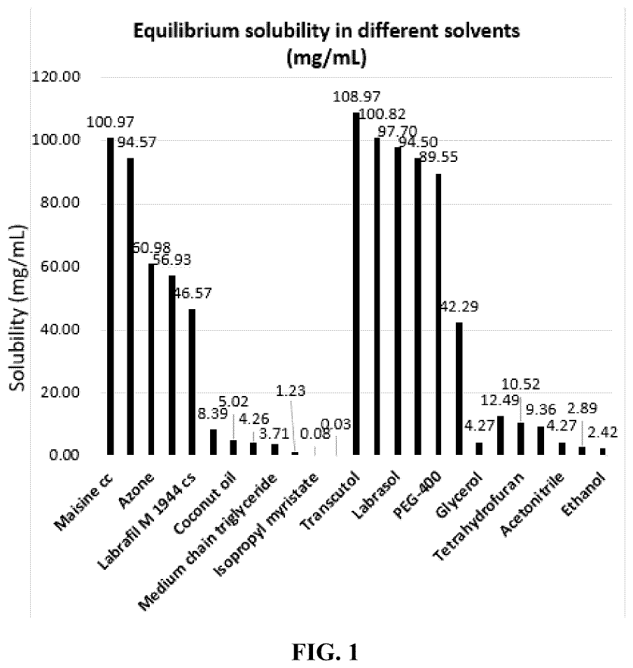

Many different solvents were evaluated for their compatibility with midostaurin. The method of solubility measurement includes adding excess amorphous midostaurin powders into various solvents listed on the x axis of FIG. 1 and mixing for 24-72 hours at 25° C. with 600 rpm to ensure equilibrium was achieved. Solubility was measured by HPLC. The HPLC methods and parameters are provided in Table A-1.

TABLE A-1
HPLC method for measuring midostaurin assay content.

| Column | C18, 5 μm, 4.6 × 150 mm | | |
|---|---|---|---|
| Wavelength | 294 nm | | |
| Column temperature | 40° C. | | |
| Flow rate | 1 mL/min | | |
| Injection volume | 10 μL | | |
| | Time (min) | A % | B % |
| Mobile phase | 0 | 90 | 10 |
| A: 0.05% TFA/water; | 3 | 90 | 10 |
| B: Acetonitrile | 7 | 20 | 80 |
| | 10 | 20 | 80 |
| | 10.1 | 90 | 10 |
| | 13 | 90 | 10 |

The results from FIG. 1 show that midostaurin exhibited low solubility (<10 mg/mL) in many hydrophilic solvents, such as ethanol and glycerol in the current study. Midostaurin has relatively high hydrophobicity, with a log P>5, suggesting it may have higher solubility in lipid solvents. However, the results showed low solubility (<10 mg/mL) in most oils, such as soybean oil and corn oil. Midostaurin has good solubility (>50 mg/mL) in certain solubilizers, such as Tween 80 and Labrasol.

The solubility of midostaurin in a system with water and a solubilizer combined at a certain ratio was also evaluated. The method involves adding excess amorphous midostaurin powders into various concentrations of solubilizer and water systems and mixed for 24 hours at 25° C. with 600 rpm to ensure equilibrium was achieved. Solubility was measured by HPLC using the method provided in Table A-1. Solubilizers selected for this study include Transcutol, Labrasol, Tween 80, Cremphor EL, and Kolliphor HS15. Each solubilizer is mixed with water at 10%, 20%, and 30% v/v, respectively, to form a system where amorphous midostaurin power was dissolved.

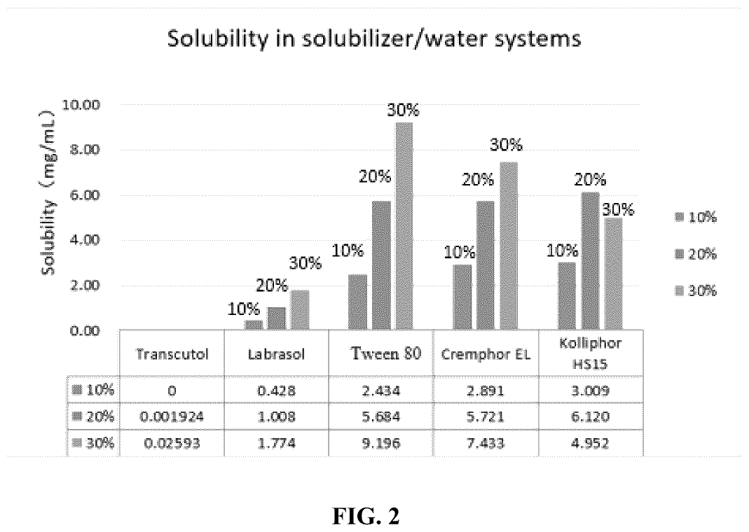

The results are summarized in FIG. 2. Although Tween 80, Cremphor EL and Kolliphor could significantly increase the solubility of midostaurin in water, all of the systems tested still show low solubility (<10 mg/mL).

### Example B

### Excipient Compatibility Study for Midostaurin

This example illustrates the process of selecting suitable excipients for a topical formulation for midostaurin or a pharmaceutically acceptable salt thereof, according to some embodiments of the present disclosure.

Midostaurin was mixed with many excipients used in topical formulations at 1:200 (w/w) and was placed at 60° C. for 10 days before testing. Impurities for each API and excipient combination were measured by HPLC, and results are listed in Table B-1. The methods and parameters for HPLC arc provided in Table B-2.

TABLE B-1
The impurity results for midostaurin mixed with various excipients used
in topical formulations at 1:200 (w/w) and placed at 60° C. for 10 days.

| | Impurity results (%) | | | | | | | | | | | |
|---|---|---|---|---|---|---|---|---|---|---|---|---|
| | RRT | | | | | | | | | | | |
| Samples | 0.41 | 0.46 | 0.52 | 0.59 | 0.62 | 0.66 | 0.68 | 0.70 | 0.76 | 0.86 | 0.91 | 0.93 |
| API | — | — | — | — | — | — | — | — | — | — | 0.07 | 0.11 |
| API + | — | 2.07 | 1.69 | — | 0.88 | — | — | 0.96 | 0.15 | 0.12 | — | — |
| Peceol | | | | | | | | | | | | |
| API + | 0.51 | 0.77 | — | 0.56 | — | 0.55 | — | — | — | 0.41 | — | — |
| Maisine | | | | | | | | | | | | |
| CC | | | | | | | | | | | | |
| API + | 0.13 | 0.05 | 0.12 | 0.17 | — | 0.09 | — | — | 0.03 | 0.03 | — | — |
| Plurol | | | | | | | | | | | | |
| oleique | | | | | | | | | | | | |
| CC 497 | | | | | | | | | | | | |
| API + | — | 0.61 | 0.08 | 0.51 | — | 0.15 | 0.44 | — | — | 0.52 | — | — |
| Labrafil | | | | | | | | | | | | |
| M | | | | | | | | | | | | |
| 1944CS | | | | | | | | | | | | |
| API + | — | 1.15 | 0.07 | 2.91 | — | — | 3.08 | — | 0.70 | 3.18 | — | — |
| Transcutol | | | | | | | | | | | | |
| API + | — | 0.78 | — | 2.44 | — | — | 1.87 | — | — | 1.96 | — | — |
| Tween 80 | | | | | | | | | | | | |
| API + | — | 0.06 | 0.09 | 0.07 | — | 0.07 | — | — | 0.28 | 2.91 | — | 1.93 |
| Peceol + | | | | | | | | | | | | |
| Tofse 63 | | | | | | | | | | | | |
| API + | — | 0.50 | 0.11 | 1.50 | — | — | 1.08 | — | 0.07 | 2.67 | — | — |
| Peceol + | | | | | | | | | | | | |
| Gelot 64 | | | | | | | | | | | | |
| API + | — | 0.99 | 0.27 | 0.58 | 0.04 | — | 0.19 | — | 0.09 | 0.10 | — | — |
| Peceol + | | | | | | | | | | | | |
| Span 60 | | | | | | | | | | | | |
| API + | — | 0.54 | 0.18 | 0.86 | — | — | 0.68 | — | 0.08 | 0.05 | — | 0.05 |
| Peceol + | | | | | | | | | | | | |
| BHA | | | | | | | | | | | | |
| API + | — | 2.32 | — | — | — | — | — | — | — | 0.04 | — | — |
| Propylene | | | | | | | | | | | | |
| glycol | | | | | | | | | | | | |
| API + | — | — | 0.18 | 0.86 | — | — | 0.69 | — | 0.08 | 0.05 | — | 0.05 |
| Glycerol | | | | | | | | | | | | |
| API + | — | 3.45 | — | 1.30 | — | — | 1.25 | — | 0.06 | 1.44 | — | — |
| PEG 400 | | | | | | | | | | | | |
| API + | — | 0.40 | 0.08 | 0.14 | 0.37 | — | 0.52 | — | 0.09 | 0.75 | — | — |
| PEG 4000 | | | | | | | | | | | | |

| | Impurity results (%) | | | | | | | | |
|---|---|---|---|---|---|---|---|---|---|
| | RRT | | | | | | | | |
| Samples | 0.97 | 1.00 | 1.10 | 1.23 | 1.25 | 1.34 | 1.51 | 1.62 | 1.76 |
| API | 0.12 | 99.64 | 0.06 | — | — | — | — | — | — |
| API + | — | 88.83 | 1.64 | — | — | — | — | — | 3.68 |
| Peceol | | | | | | | | | |
| API + | — | 88.82 | 2.02 | — | — | — | — | — | 6.38 |
| Maisine | | | | | | | | | |
| CC | | | | | | | | | |
| API + | — | 99.27 | — | 0.02 | — | 0.05 | — | — | — |
| Plurol | | | | | | | | | |
| oleique | | | | | | | | | |
| CC 497 | | | | | | | | | |
| API + | — | 95.95 | 0.34 | — | — | 0.08 | — | 0.04 | 1.28 |
| Labrafil | | | | | | | | | |
| M | | | | | | | | | |
| 1944CS | | | | | | | | | |
| API + | — | 65.90 | 5.93 | 2.06 | — | 2.24 | 12.75 | — | 12.75 |
| Transcutol | | | | | | | | | |
| API + | — | 80.83 | — | — | — | — | 0.15 | — | 10.76 |
| Tween 80 | | | | | | | | | |
| API + | — | 85.62 | 2.32 | 0.16 | 0.19 | — | — | 1.94 | 4.49 |
| Peceol + | | | | | | | | | |
| Tofse 63 | | | | | | | | | |
| API + | — | 86.19 | 0.58 | — | — | — | 0.16 | — | 7.15 |
| Peceol + | | | | | | | | | |
| Gelot 64 | | | | | | | | | |
| API + | — | 91.96 | 0.05 | — | — | — | — | — | 5.75 |
| Peceol + | | | | | | | | | |
| Span 60 | | | | | | | | | |
| API + | — | 95.35 | 1.58 | — | — | — | — | — | 0.64 |
| Peceol + | | | | | | | | | |
| BHA | | | | | | | | | |
| API + | — | 97.64 | — | — | — | — | — | — | — |
| Propylene | | | | | | | | | |
| glycol | | | | | | | | | |
| API + | — | 95.87 | 1.58 | — | — | — | — | — | 0.64 |
| Glycerol | | | | | | | | | |
| API + | — | 82.09 | 0.34 | — | — | — | 0.62 | — | 9.44 |
| PEG 400 | | | | | | | | | |
| API + | — | 76.01 | 4.97 | — | — | — | 0.61 | — | 16.04 |
| PEG 4000 | | | | | | | | | |

TABLE B-2
HPLC method for measuring midostaurin and its impurities.

| | |
|---|---|
| Column | C18, 5 μm, 4.6 × 150 mm |
| Wavelength | 294 nm |
| Column temperature | 40° C. |
| Flow rate | 0.9 mL/min |
| Injection volume | 10 μL |
| Mobile phase | 40 mM ammonium formate:Acetonitrile (35:65, v/v) |

The excipient compatibility results show that midostaurin powder was very stable at 60° C. for 10 days (assay content is 99.64%). However, when dissolved in different excipients, it became less stable, with the biggest impurity appearing at RRT 1.76. Less degradation was seen when antioxidant (e.g., butylated hydroxyanisole) was added to the mixture of midostaurin and Peceol. One possibility is that when midostaurin is dissolved in oil, it is easier to produce oxidized impurities when exposed to high temperature.

### Example C

### Method of Skin Deposition Measurement to Evaluate the Efficiency of Various Topical Formulation for Midostaurin to Penetrate Skin Layers

This example illustrates the steps for evaluating how much midostaurin can penetrated the skin barrier when midostaurin was prepared in a specific topical formulation, according to some embodiments of the present disclosure.

Melasma is clinically characterized by epidermal hyperpigmentation. The histopathological changes involve both the epidermis and dermis, as illustrated in FIG. 3. Therefore, topically administered midostaurin should ideally penetrate into the deeper skin layer. However, the outermost layer of skin, the stratum corneum (SC), can be a barrier for drug penetration. A skin penetration enhancer can be designed into topical formulations to help midostaurin penetrates the SC. A skin deposition test may be used to evaluate the penetration efficiency.

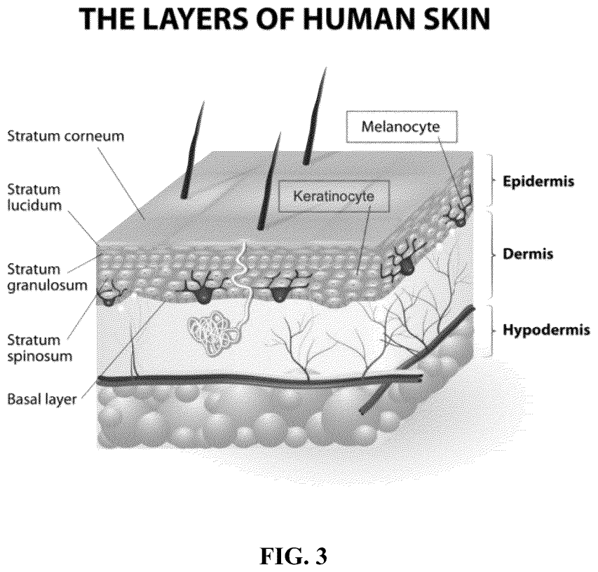

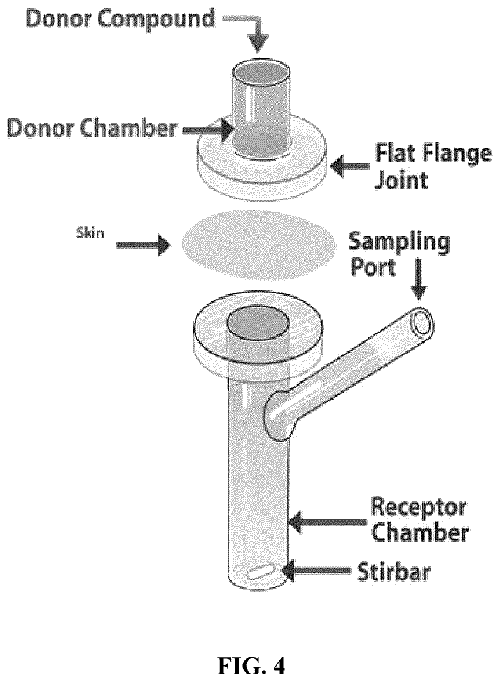

Skin deposition or retention experiments are performed ex-vivo using Franz diffusion cells, as illustrated in FIG. 4, and nude mice skin. The skin of nude mice (˜6-week-old) was carefully excised, cleaned and mounted on the diffusion cell with the stratum corneum facing the donor side. The tested formulation is typically added to the donor compartment and incubated with the skin for a predetermined interval at a constant temperature (32±0.5° C.). At the end of the experiment, the skin was removed, washed and the stratum corneum was removed using tape stripping procedure. The midostaurin content in the stratum corneum layer and epidermis/dermis layers were determined by HPLC method described in Table A-1.

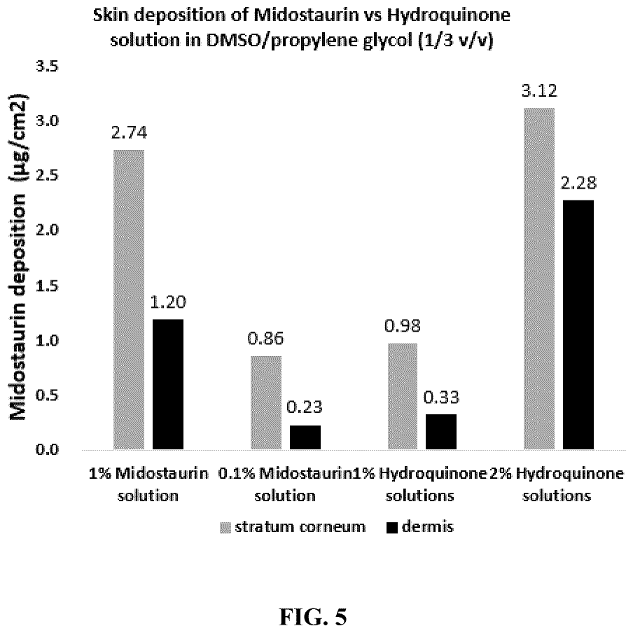

A series of midostaurin solutions (0.1%, and 1%), as well as Hydroquinone solutions (1%, 2%) were prepared in a mixture of DMSO and propylene glycol (1:3, v/v). The deposition amount of midostaurin and Hydroquinone in nude mouse skins were investigated. The results are summarized in FIG. 5 and Table C-1 and indicate that skin deposition of Hydroquinone at conventional concentration (2%) was greater than midostaurin at 1% when they are dissolved in the same solution. Thus, formulation strategy should be considered to enhance the permeability of midostaurin in skin.

TABLE C-1
Skin deposition of Midostaurin vs Hydroquinone
solution in DMSO/propylene glycol (1/3 v/v)

| | | Sample | Sample | Sample | |
|---|---|---|---|---|---|
| API deposition (μg/cm²) | | 1 | 2 | 3 | Average |
| 1% Midostaurin | stratum | 1.53 | 5.07 | 1.61 | 2.74 |
| solution | corneum | | | | |
| | dermis | 0.42 | 1.86 | 1.31 | 1.20 |
| 0.1% Midostaurin | stratum | 0.92 | 0.49 | 1.18 | 0.86 |
| solution | corneum | | | | |
| | dermis | 0.18 | 0.10 | 0.43 | 0.23 |
| 1% Hydroquinone | stratum | 0.24 | 1.00 | 1.69 | 0.98 |
| solutions | corneum | | | | |
| | dermis | 0.06 | N/A | 0.59 | 0.33 |
| 2% Hydroquinone | stratum | 3.13 | 1.58 | 4.65 | 3.12 |
| solution | corneum | | | | |
| | dermis | 4.94 | 1.05 | 0.84 | 2.28 |

### Example D

### Development of Midostaurin Gel Formulation

This example illustrates the process of developing gel formulation for midostaurin, according to some embodiments of the present disclosure.

Transcutol, Labrasol, Tween 80, or a combination thereof were used to prepare the gel formulations as shown in Table D-1, where each component and its percentage by weight were listed. The skin deposition in stratum and dermis for the gel formulations were determined using the method described in Example C.

TABLE D-1
Gel formulations for midostaurin.

| | Lot A1 | Lot A2 | Lot A3 | Lot A4 |
|---|---|---|---|---|
| | Percentage | Percentage | Percentage | Percentage |
| Component | (wt %) | (wt %) | (wt %) | (wt %) |
| Midostaurin | 0.1 | 0.1 | 0.1 | 0.1 |
| Carbomer 940 | 0.5 | 0.5 | 0.5 | 0.5 |
| Transcutol | 10 | — | — | 20 |
| Labrasol | — | 10 | — | 10 |
| Tween 80 | — | — | 10 | — |
| Triethanol- | pH adjusted | pH adjusted | pH adjusted | pH adjusted |
| amine | to 7.0 | to 7.0 | to 7.0 | to 7.0 |
| Purified water | To 100 | To 100 | To 100 | To 100 |

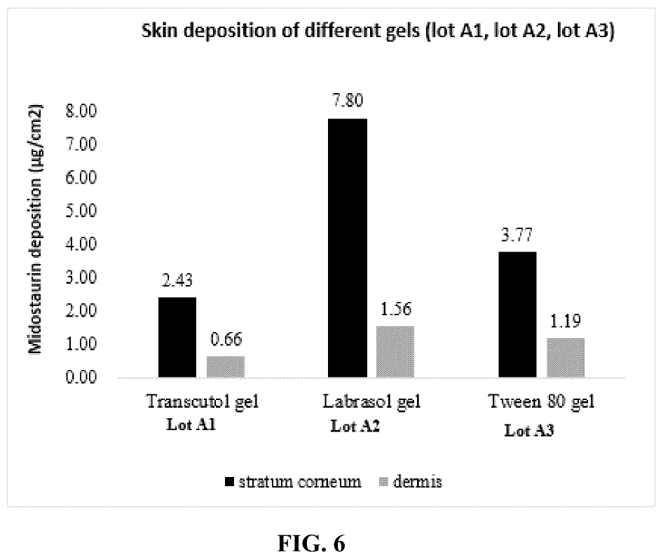

The skin deposition results for lots A1, A2, and A3 are listed in FIG. 6. Gel formulation with Labrasol (lot A2) has the most skin deposition.

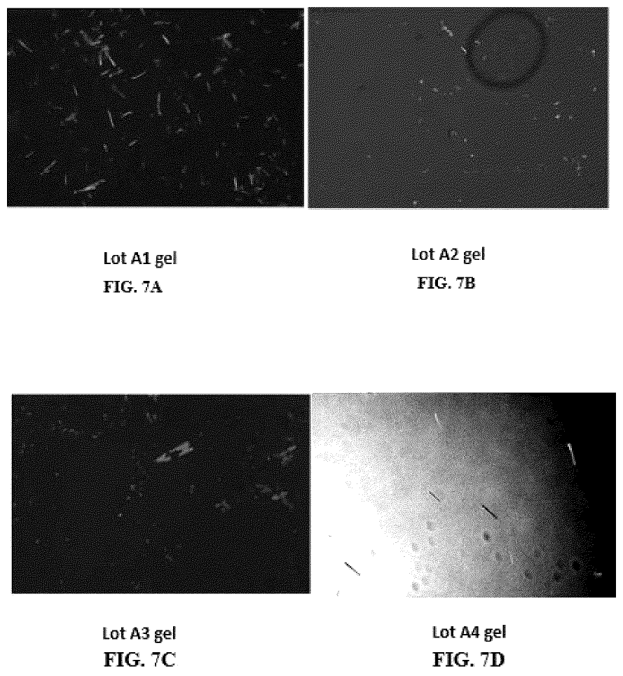

Since gel formulations tend to crystallize, lots A1, A2, A3, and A4 were placed at 60° C. for 7 days to investigate their crystallization under polarization microscope. The results for crystallization under polarization microscope were shown in FIGS. 7A-7D. The micrographs show needle-like and star-like crystallization of midostaurin in the gel formulation, especially in lot A1 gel. It appears that gel formulations tend to crystallize after being exposed to 60° C. regardless of whether a single solubilizer at 10% w/w was used or mixed solubilizers at a level as high as 30% w/w were used. Since midostaurin has relatively low solubility in a solubilizer and water system, the gel formulation development can be challenging. Cream and ointment formulations can be considered for next steps.

### Example E

### Development of Midostaurin Cream Formulation

This example illustrates the process of developing cream formulation for midostaurin, according to some embodiments of the present disclosure.

A cream formulation for midostaurin cream (lot B1) was prepared and appropriate amount of Maisine CC was additionally added (lot B2) as oil phase to observe the improvement of crystallization.

The complete formulation for lot B1 and B2 is shown in Table E-1.

TABLE E-1
Cream formulations for midostaurin - lots B1 and B2

| | Lot B1 | Lot B2 |
|---|---|---|
| Component | Percentage (wt %) | Percentage (wt %) |
| Midostaurin | 1.0 | 1.0 |
| Octadecyl alcohol | 3.5 | 3.5 |
| White Vaseline | 3 | 3 |
| Liquid paraffin | 5 | 5 |
| Maisine CC | — | 3 |
| Glyceryl monostearate | 3 | 3 |
| Sodium dodecyl sulfate | 1 | 1 |
| Transcutol | 10 | 10 |
| Propylene glycol | 15 | 15 |
| Glycerin | 5 | 5 |
| Purified water | to 100 | to 100 |

Preparation procedures for the cream formulation involve preparing an oil phase and an aqueous phase. To prepare the oil phase, combine weighted API, octadecyl alcohol, white Vaseline, liquid paraffin, glyceryl monostearate, Transcutol and/or Maisine CC into the same beaker, heat to above 80° C. for complete melting. To prepare the aqueous phase, combine weighted sodium dodecyl sulfate, propylene glycol, glycerin and water into the same beaker and heat to above 80° C. Gradually add the oil phase into the aqueous phase under continuous stirring until cream was formed.

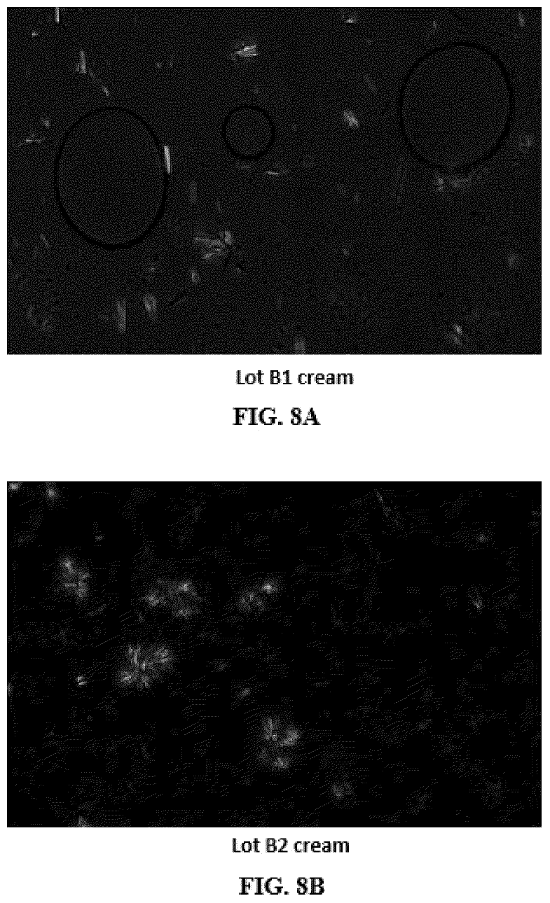

Both lots B1 and B2 were placed at 60° C. for continuous investigation of crystallization under polarization microscope, and the results are shown in FIGS. 8A-8B. The micrographs show needle-like and snowflake-like crystallization of midostaurin in the cream formulation. Both creams (lot B1 and B2) can be kept stable at room temperature for first 2 weeks, and crystallization started from week 3. The main cause of crystallization may be insufficient solubility of midostaurin in oil phase. To prevent crystallization of midostaurin in a cream formulation at the concentration of 1%, the oil content should be not less than 10% according to the solubility of midostaurin in various oil as shown in FIG. 1.

To investigate the stability of the cream formulation, midostaurin cream (lot B1) was exposed to 4500 Lx and 60° C. for 7 days and went through a freeze-thaw cycle (60° C., 6 h/−20° C., 24 h). The impurities were tested using the HPLC methods as described in Table B-2. The impurity results are summarized in Table E-2. The data shows that Midostaurin cream (lot B1) was sensitive to light exposure, thus, steps to avoid light exposure or add sun-screening agent can be considered in the subsequent formulation development. High temperature and freeze-thaw cycle had less effect on its stability.

TABLE E-2
Impurity results for midostaurin cream (lot B1)
Impurity results (%)

| | RRT | | | | | | | |
|---|---|---|---|---|---|---|---|---|
| Conditions | 0.23 | 0.45 | 0.54 | 0.59 | 0.68 | 0.75 | 1.00 | 1.77 |
| 60° C. | 0.11 | 0.12 | 0.12 | 0.13 | 0.12 | 0.05 | 99.33 | — |
| freeze- | 0.10 | 0.11 | 0.11 | 0.14 | 0.08 | 0.06 | 99.38 | — |
| thaw cycle | | | | | | | | |
| 4500 lx | 0.11 | 0.09 | 0.13 | 0.74 | 0.70 | 0.14 | 97.61 | 0.48 |

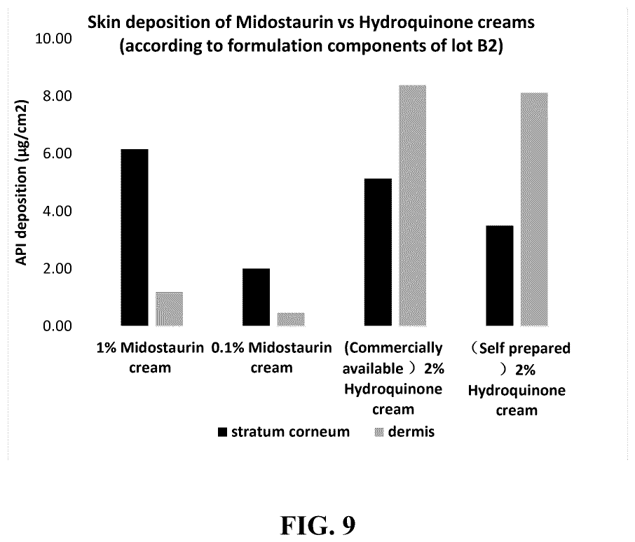

To investigate the skin deposition of midostaurin in cream formulation, 0.1% and 1% midostaurin cream, as well as 2% hydroquinone cream were prepared according to the formulation components of lot B2. In addition, a commercially available 2% hydroquinone cream was also tested for comparison. The skin deposition test was performed for the four creams according to the steps described in Example C. Results are summarized in FIG. 9 and Table E-3. The data suggested that 2% hydroquinone cream, both self-prepared and commercial, showed better skin deposition than midostaurin cream formulations, which can be attribute to the relatively lower log P for hydroquinone (1.09 vs. 5.27). Midostaurin cream formulations show higher deposition of midostaurin in stratum corneum than that of midostaurin solution (1% midostaurin; 6.16 μg/cm² in cream vs. 2.74 μg/cm² in solution; 0.1% midostaurin; 2.01 μg/cm² in cream vs. 0.86 μg/cm² in solution). However, cream formulation had little improvement for deposition of midostaurin in dermis when compared to midostaurin solution (1%; 1.20 μg/cm² for cream vs. 1.20 μg/cm² for solution; 0.1%; 0.47 μg/cm² for cream vs. 0.23 μg/cm² for solution). Penetration enhancer can be considered to further increase the deposition of midostaurin the skin.

TABLE E-3
Skin deposition of midostaurin creams and hydroquinone creams

| | | Sample | Sample | Sample | |
|---|---|---|---|---|---|
| API deposition (μg/cm²) | | 1 | 2 | 3 | Average |
| 1% Midostaurin | stratum | 4.67 | 11.22 | 2.58 | 6.16 |
| cream | corneum | | | | |
| | dermis | 1.05 | 1.19 | 1.37 | 1.20 |
| 0.1% Midostaurin | stratum | 2.55 | 0.94 | 2.55 | 2.01 |
| cream | corneum | | | | |
| | dermis | 0.44 | 0.61 | 0.37 | 0.47 |
| 2% Hydroquinone | stratum | 5.31 | 2.97 | 7.12 | 5.13 |
| cream (commercial) | corneum | | | | |
| | dermis | 13.01 | 4.91 | 7.23 | 8.38 |
| 2% Hydroquinone | stratum | 4.56 | 2.59 | 3.34 | 3.50 |
| cream (self- | corneum | | | | |
| prepared) | dermis | 4.60 | 11.93 | 7.83 | 8.12 |

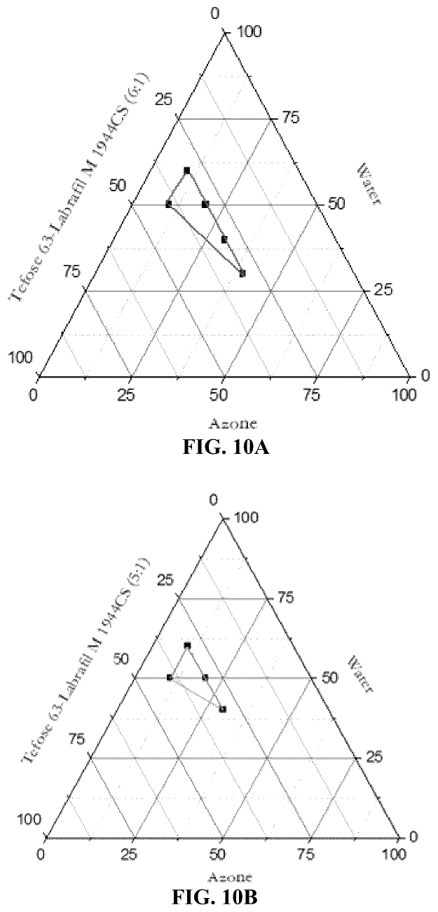

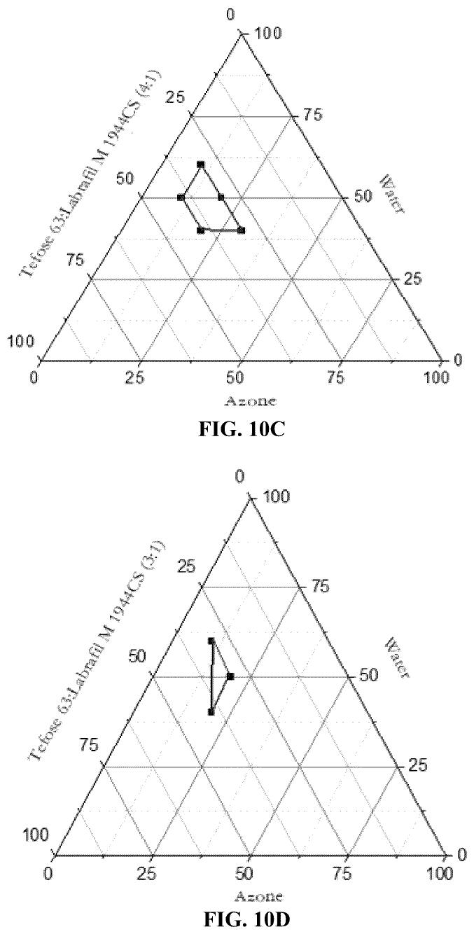

Azone was used in oil phase and Tefose 63 and Labrafil M 1944CS were selected as co-emulsifiers. Four ternary phase diagrams were prepared, as shown in FIGS. 10A-10D. The designated area in the ternary phase diagrams suggest a range for oil phase, water phase, and emulsifier so that a cream can be formed, and each point in this area corresponds to a certain percentage of oil phase, water phase and emulsifier respectively. According to the results of the ternary phase diagrams, appropriate percentages of oil phase, water phase and emulsifier of the cream were selected. Optimum oil and co-emulsifiers ratios were selected to prepare 1% midostaurin creams. Complete formulations are listed in Table E-4.

TABLE E-4
Cream formulations (lots B3, B4, B5, B6, and B7)

| | Percentage (wt %) | | | | |
|---|---|---|---|---|---|
| Component | Lot B3 | Lot B4 | Lot B5 | Lot B6 | Lot B7 |
| Tefose 63 | 25 | 33.33 | 25 | 25 | 26.66 |
| Labrafil M 1944CS | 5 | 6.66 | 5 | 5 | 13.33 |
| Azone | 20 | 10 | 10 | 30 | 20 |
| Purified Water | 49 | 49 | 59 | 39 | 39 |
| Midostaurin | 1 | 1 | 1 | 1 | 1 |

To prepare the oil phase, API, Azone, Tefose 63 and Labrafil M 1944CS were weighed and added into the same beaker, heated to above 80° C. for complete melting. To prepare the aqueous phase, water was weighed into a beaker and heated to above 80° C. Then, gradually add the oil phase to the aqueous phase and keep continuous stirring until cream was formed. Lots B3 and B7 were placed at 4500 1x, 60° C., or room temperature for 7 days for a stress test. The midostaurin content was measured using HPLC method described in Table B-2, and the results are shown in Table E-5. The assay results suggest significant degradation of midostaurin in lots B3 and B7. Oxidation impurities may be produced due to conditions such as high temperature, light or a large amount of oil.

TABLE E-5
Stress Test for Cream formulations (lots B3, B7)
Midostaurin cream stress testing results (Assay %)

| Conditions | B3 | B7 |
|---|---|---|
| 4500Lx | 91.86 | 94.91 |
| 60° C. | 91.99 | 95.57 |
| Room temperature | 99.40 | 99.52 |

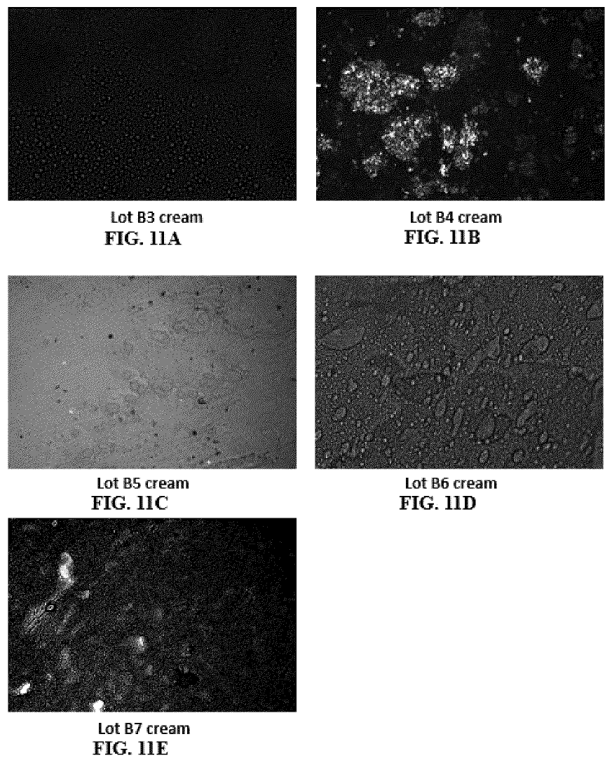

Lots B3, B4, B5, B6, and B7 were placed at 60° C. to investigate their crystallization under polarization microscope. Results are shown in FIGS. 11A-11E. The micrographs of lots B3 and B7 shows that the droplets are evenly distributed, without needle like or star like crystallization. Lots B3 and B7 did not show any crystallization under 60° C. for 7 days. The micrographs of lots B4 and B5 show that the star-like crystallization occurred. Lots B4 and B5 showed crystallizations when placed at room temperature after 3 days. For lot B6, demulsification was observed when placed at room temperature. The micrograph of lot B6 shows that the droplet distribution became wider, and the droplet diameter became larger.

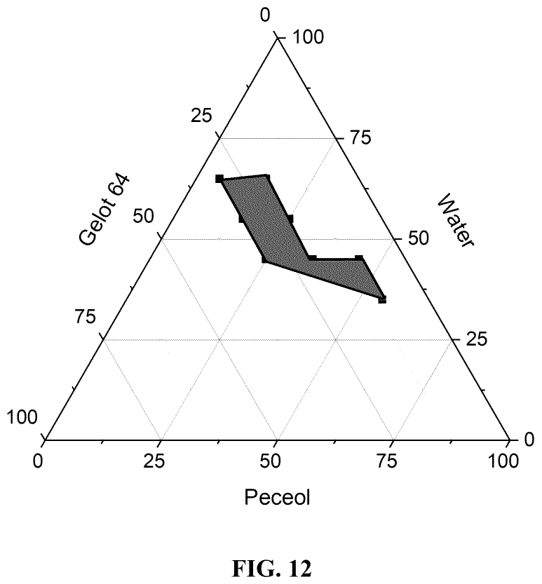

Peceol was selected as the oil phase and Gelot 64 was selected as emulsifiers, a ternary phase diagram was prepared as shown in FIG. 12. Optimum oil and co-emulsifiers ratios were selected to prepare 1% midostaurin cream. The formulation is listed in Table E-6.

TABLE E-6
Cream formulation (lot B8)

| Component | Percentage (wt %) |
|---|---|
| Gelot 64 | 20 |
| Peceol | 30 |
| Purified water | 49 |
| Midostaurin | 1 |

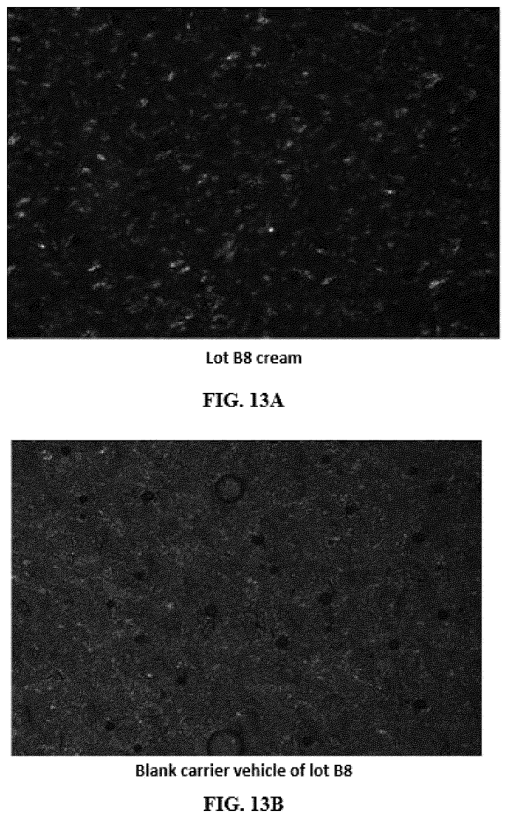

To prepare the oil phase, API, Peceol, and Gelot 64 were weighted into the same beaker and heated to above 80° C. for complete melting. To prepare the aqueous phase, water was weighed into a beaker and heated to above 80° C. Then, gradually add the oil phase to the aqueous phase and stir it into cream. The cream was placed in 4500 lx, 60° C. and room temperature for 7 days. The midostaurin impurities were measured using HPLC method described in Table B-2, and the results are shown in Table E-7. Midostaurin cream prepared with Peceol/Gelot 64 was very susceptible to high temperature, which appears to be consistent with the hypothesis that oxidation impurities are more likely to occur under high temperature and a larger amount of oil. Lot B8 and a blank carrier vehicle of lot B8 were placed at 60° C. for 7 days to investigate their crystallization under polarization microscope and the results are shown in FIGS. 13A-13B. The micrograph of lot B8 is similar to that of blank carrier vehicle, indicating that it is in a stable state. Lot B8 cream did not show crystallization under 60° C. for 7 days, so it was concluded that there was no crystal precipitation in midostaurin cream for the time duration evaluated.

TABLE E-7
Stress test for cream formulation (lot B8)
Impurity results (%)

| | RRT | | | | | | | | |
|---|---|---|---|---|---|---|---|---|---|
| Conditions | 0.47 | 0.50 | 0.56 | 0.60 | 0.67 | 0.76 | 1.00 | 1.11 | 1.77 |
| Room temperature | 0.05 | — | — | 0.09 | 0.19 | 0.27 | 98.77 | 10.32 | 0.33 |
| 60° C. | 0.24 | 0.05 | — | 0.54 | 0.72 | 1.00 | 90.61 | 4.50 | 2.75 |
| 4500 Lx | 0.12 | 0.01 | 0.10 | 0.07 | 0.38 | 0.38 | 98.50 | — | 0.44 |

According to the optimized cream formulation with Peceol as the oil phase, midostaurin cream was prepared with penetration enhancers and optionally, antioxidants, added to the formulation. First, the cream formed with different ratios of Peceol, emulsifiers (Tween 80 and Span 60 combined), and water, as listed in Table E-8, were compared. When emulsifiers are about 20% and oil phase was about 10%, the cream has good stability, no-oil feeling and smooth.

TABLE E-8
Different ratios of Peceol, Tween 80, Span
60, and water for cream formulation.

| Peceol (wt %) | Tween 80(wt %) | Span 60 (wt %) | Water (wt %) |
|---|---|---|---|
| 10 | 16 | 4 | 70 |
| 10 | 24 | 6 | 60 |
| 15 | 16 | 4 | 65 |
| 15 | 24 | 6 | 55 |
| 20 | 8 | 2 | 70 |
| 20 | 16 | 4 | 60 |
| 20 | 24 | 6 | 50 |
| 25 | 16 | 4 | 55 |
| 30 | 8 | 2 | 60 |

To prepare the oil phase, weigh API, Peceol, Tween 80, Span 60, antioxidants, and one of the three penetration enhancers, Plurol oleique CC 497, Transcutol, or Labrafil M 1944 CS into the same beaker, heat them to above 80° C. for melting. To prepare the aqueous phase, weigh water into a beaker and heat to above 80° C. Gradually add the oil phase to the aqueous phase and stir it into cream. Different penetration enhancers (all at level of 5%) and different antioxidants (all at level of 0.02%) were compared. The full formulation is shown in Table E-9.

TABLE E-9
Cream formulations (lot B9, B10, B11, B12, B13, B14, and B15)

| Component (wt %) | | B9 | B10 | B11 | B12 | B13 | B14 | B15 |
|---|---|---|---|---|---|---|---|---|
| Peceol | | 10 | 10 | 10 | 10 | 10 | 10 | 10 |
| Tween 80 | | 16 | 16 | 16 | 14 | 14 | 14 | 14 |
| Span 60 | | 4 | 4 | 4 | 3.5 | 3.5 | 3.5 | 3.5 |
| Plurol oleique | | 5 | — | — | 5 | 5 | 5 | 5 |
| CC 497 | | | | | | | | |
| Transcutol | | — | 5 | — | — | — | — | — |
| Labrafil M 1944 CS | | — | | 5 | — | — | — | — |
| Midostaurin | | 1 | 1 | 1 | 1 | 1 | 1 | 1 |
| Antioxidants | BHT | — | — | — | 0.02 | — | — | — |
| | BHA | — | — | — | — | 0.02 | — | — |
| | Ascorbyl Palmitate | — | — | — | — | — | 0.02 | — |

| | | | | | | | |
|---|---|---|---|---|---|---|---|
| Purified water | to 100 | to 100 | to 100 | to 100 | to 100 | to 100 | to 100 |

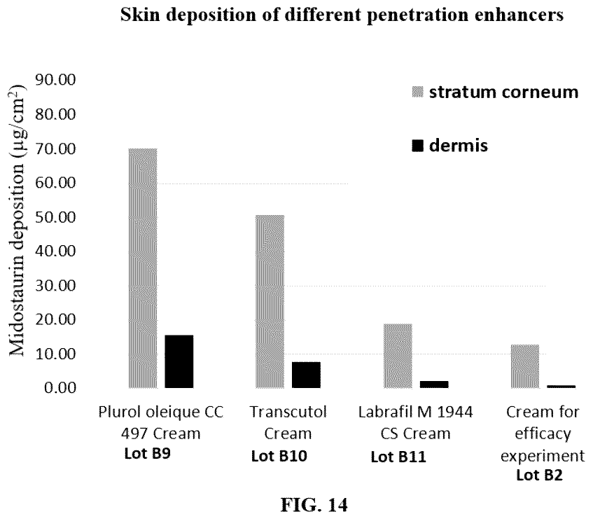

Skin deposition test was performed for formulations (lot B9, B10, B11) using different penetration enhancers, the results are shown in FIG. 14 and Table E-10. The cream formulations above (lot B11, B12, B13, B14) were exposed to 4500 Lx, 40° C., 60° C. or placed at room temperature for 7 days as a stress test. The midostaurin impurities were measured using HPLC method described in Table B-2, and the results are shown in Table E-11. Plurol oleique CC 497 produced the best penetration-promoting effect. BHT showed the best antioxidant effect.

TABLE E-10
Skin deposition of different penetration enhancers

| | | Sample | Sample | Sample | |
|---|---|---|---|---|---|
| Midostaurin deposition (μg/cm²) | | 1 | 2 | 3 | Average |
| Plurol oleique | stratum | 52.86 | 84.44 | 73.22 | 70.18 |
| CC 497 (B9) | corneum | | | | |
| | dermis | 17.91 | 16.64 | 12.09 | 15.55 |
| Transcutol (B10) | stratum | 64.05 | 32.35 | 55.31 | 50.57 |
| | corneum | | | | |
| | dermis | 9.57 | 4.30 | 9.07 | 7.65 |
| Labrafil M | stratum | 21.50 | 18.54 | 16.07 | 18.70 |
| 1944 CS (B11) | corneum | | | | |
| | dermis | 3.77 | 1.52 | 0.88 | 2.06 |
| Cream for PD | stratum | 12.00 | 13.97 | 12.31 | 12.76 |
| experiment (B2) | corneum | | | | |
| | dermis | 0.75 | 1.30 | 0.66 | 0.90 |

TABLE E-11
Impurity results for several antioxidants added to the midostaurin cream (lots B12, B13, B14, and B15)

| | | Impurity results (%) | | | | | | | | | | | | | | |
|---|---|---|---|---|---|---|---|---|---|---|---|---|---|---|---|---|
| | | RRT | | | | | | | | | | | | | | |
| Antioxidants | | 0.37 | 0.39 | 0.45 | 0.48 | 0.55 | 0.61 | 0.68 | 0.69 | 0.76 | 0.85 | 0.94 | 1.00 | 1.10 | 1.30 | 1.77 |
| BHA (lot | RT | — | — | 0.28 | — | 0.03 | 0.05 | — | 0.04 | — | — | — | 99.60 | — | — | — |
| B12) | 40° C. | — | — | 0.32 | — | 0.03 | 0.06 | — | 0.06 | 0.02 | 0.05 | — | 99.47 | — | — | — |
| | 60° C. | — | — | 0.51 | — | 0.07 | 0.14 | — | 0.18 | 0.04 | 0.13 | — | 98.71 | — | — | 0.15 |
| | 4500 lx | 0.06 | — | 0.54 | — | 0.36 | 1.51 | — | 1.71 | 0.27 | — | — | 95.20 | 0.06 | 0.17 | 0.12 |
| BHT (lot | RT | — | — | 0.27 | — | 0.03 | 0.05 | — | 0.38 | 0.10 | — | — | 99.17 | — | — | — |
| B13) | 40° C. | — | — | 0.21 | — | 0.03 | 0.06 | — | 0.45 | 0.04 | 0.06 | — | 99.07 | — | — | 0.04 |
| | 60° C. | — | — | 0.32 | — | 0.03 | 0.18 | — | 0.57 | 0.08 | 0.16 | — | 98.47 | — | — | 0.17 |
| | 4500 lx | — | — | 0.31 | 0.12 | 0.25 | 0.00 | — | 1.00 | 0.25 | — | — | 97.31 | — | 0.05 | — |
| Ascorbyl | RT | — | — | 0.17 | — | 0.03 | 0.20 | — | 0.49 | 0.31 | — | — | 98.69 | — | — | 0.11 |
| Palmitate | 40° C. | — | — | 0.60 | — | 0.02 | 0.72 | — | 1.47 | 0.89 | 0.06 | — | 95.81 | — | — | 0.44 |
| (lot B14) | 60° C. | 0.09 | — | 0.56 | 0.27 | — | 3.01 | — | 3.40 | 0.91 | 0.29 | 0.06 | 87.76 | 0.34 | — | 3.32 |
| | 4500 lx | 0.03 | — | 0.29 | 0.14 | — | 1.97 | — | 1.73 | 0.33 | — | — | 95.14 | 0.07 | 0.20 | 0.09 |
| No | RT | — | — | 0.41 | — | — | 0.23 | — | 0.65 | 0.45 | — | — | 98.16 | — | — | 0.11 |
| antioxidants | 40° C. | — | — | 0.23 | 0.15 | — | 1.05 | 1.03 | 0.95 | 1.03 | 0.07 | — | 94.84 | 0.06 | — | 0.60 |
| (lot B15) | 60° C. | 0.49 | 0.23 | 0.40 | 0.26 | — | 10.51 | 16.10 | — | 5.14 | 0.56 | 0.52 | 41.73 | 2.93 | — | 21.14 |
| | 4500 lx | 0.04 | — | 0.36 | 0.17 | — | 2.10 | — | 1.98 | 0.37 | — | — | 94.55 | 0.09 | 0.24 | 0.11 |

### Example F

### Development of Midostaurin Ointment Formulation

This example illustrates the process of developing ointment formulation for midostaurin, according to some embodiments of the present disclosure.

PEG-400 and PEG-4000 were used as matrix to prepare midostaurin ointment due to its higher solubility in PEG-400, and Labrasol was added as the solubilizer. The ointment was stressed at 4500 Lx, 60° C., or room temperature for 7 days. The ointment formulation is shown in Table F-1. The midostaurin content was measured using HPLC method described in Table A-1, and the results are shown in Table F-2.

TABLE F-1
Ointment formulation (lot C1)

| Component | Percentage (wt %) |
|---|---|
| PEG 400 | 64.24 |
| PEG 4000 | 10.56 |
| Glycerol | 13.2 |
| Labrasol | 10 |
| Azone | 1 |
| Midostaurin | 1 |

TABLE F-2
Stress test for ointment formulation
Midostaurin ointment stressing test results (Assay %)

| Conditions | 4500 Lx | 60° C. | Room temperature |
|---|---|---|---|
| Midostaurin area (%) | 96.22 | 93.68 | 99.34 |

The results showed that midostaurin content decreased when midostaurin ointment was exposed to high temperature and 4500 Lx. There was no crystal peak in the XRPD test of midostaurin ointment, so it was determined that there was no crystallization in midostaurin ointment for the time evaluated.

### Example G

### Skin Pigmentation Reduction Efficacy Study

This example illustrates the process of evaluating the efficacy of various topical formulation for midostaurin in reducing skin pigmentation, according to some embodiments of the present disclosure.

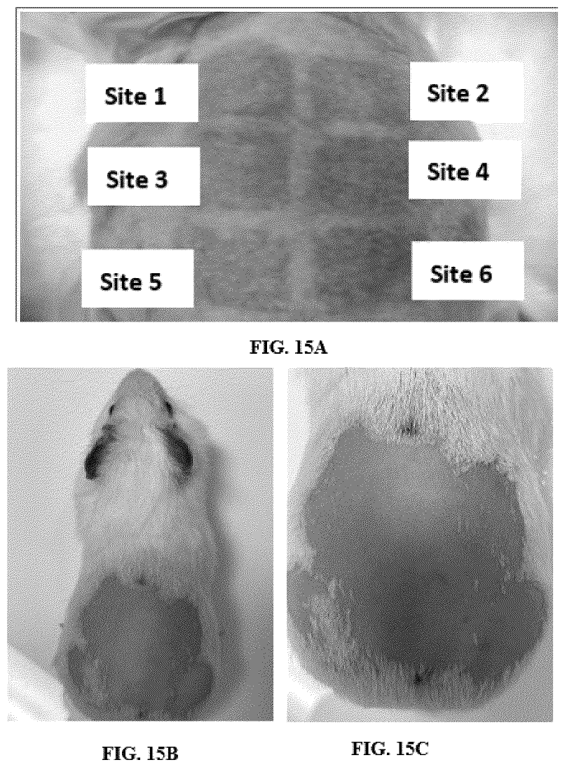

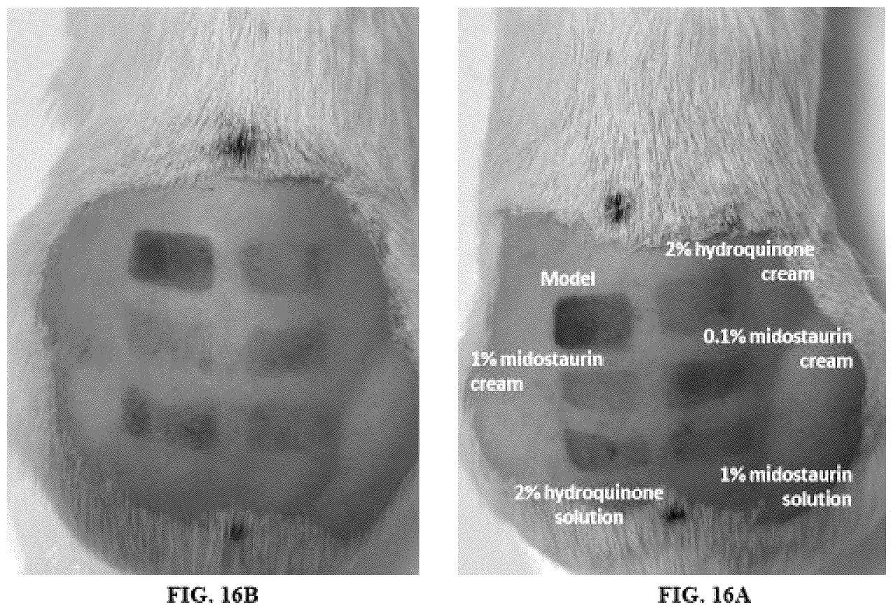

Guinea pigs that are about 12-20 weeks old are used to evaluate the efficacy of various topical formulation for midostaurin in reducing skin pigmentation. Before experiment, their backs were shaved and divided into six areas (3.2 cm^{2/}area) as illustrated in FIG. 15A. A control group (N=1) without UV irritation is shown in FIGS. 15B-C. Guinea pigs were anesthetized with 0.3 mL mixture (50 mg/mL ketamine 0.75 mL and 100 mg/mL xylazine 0.15 ml) and site 1-6 were exposed to UV light (315-400 nm) for 14 min 42 second (150 mJ/cm²) once a day consecutively for 2 weeks. The sunlight simulator contains a 1000 W xenon arc lamp equipped with a WG 305 cut-off filter. The radiation dose is 0.17 mW/cm² (or 1.7*10-4 W). Midostaurin creams (0.1% and 1.0%) were prepared according to the formulation of lot B2, and 1% Midostaurin solution and 2% Hydroquinone solution were prepared using DMSO/propylene glycol (1:3, v/v). 2% Hydroquinone cream was commercially available and was provided by Guangdong Renrenkang Pharmaceutical Co., Ltd. At the beginning of modeling, the positive control (hydroquinone cream and solutions) and the testing materials (midostaurin cream and solutions) were administrated to site 2-6 at the dose of 0.2 mL for solution or 0.2 g for cream. An equal amount of blank cream was administered to site 1, according to FIG. 16A. All treatments were administered twice a day starting from the day of UV irritation for 2 weeks. After the completion of modeling, continue to give treatments twice a day for 2 weeks, and all treatments were administered for 4 weeks in total.

The evaluation of skin pigmentation level can be done through general photographs, Fontana-Masson staining, and Tyrosinase activity measurement. For Fontana-Masson staining, the protocol and scoring standard are as follows. After the experiment, the skin tissue was taken, embedded in paraffin, and stained with Fontana silver nitrate solution. The paraffin sections stained with Fontana-Masson were examined under microscope, and the melanin particles in the skin tissue sections of each group were observed under the 200× lens. The scoring standard for local pathological observations are shown in Table G-1.

TABLE G-1
Fontana - Masson staining scoring standard
for local pathological observation.

| Score | Distribution of melanin particles |
|---|---|
| 1 | Melanin is occasionally seen in the basal layer and |
| | spinous layer of epidermis |
| 2 | Discontinuous distribution of melanin can be seen, |
| | mainly concentrated in the basement layer |
| 3 | The melanin particles are continuous distribution, |
| | mainly concentrated in the basal layer and spinous layer |
| 4 | The melanin particles are continuously distributed and |
| | concentrated in basal cells and spinous layer, and |
| | more melanin caps can be seen in keratinocytes |
| 5 | Melanin particles and melanin caps are densely |
| | distributed in the epidermis |

The protocol for tyrosinase activity measurement is as follows. The skin tissue was taken and embedded in paraffin, and immunohistochemical staining was performed. Under the 200× lens, observe the expression of tyrosinase in the slices of skin tissues, analyze the average optical density value using ImageJ software. The average optical density value is calculated by the formula AOD (average optical density)=IOD (integrated optical density)/Area of positive staining in each image.

FIGS. 16A-B show pictures of the back of a guinea pig after the study, where the skin colors are different at the six areas receiving different treatments, which are no drug treatment (model), 2% hydroquinone cream, 1% midostaurin cream, 0.1% midostaurin cream, 2% hydroquinone solution, and 1% midostaurin solution, respectively.

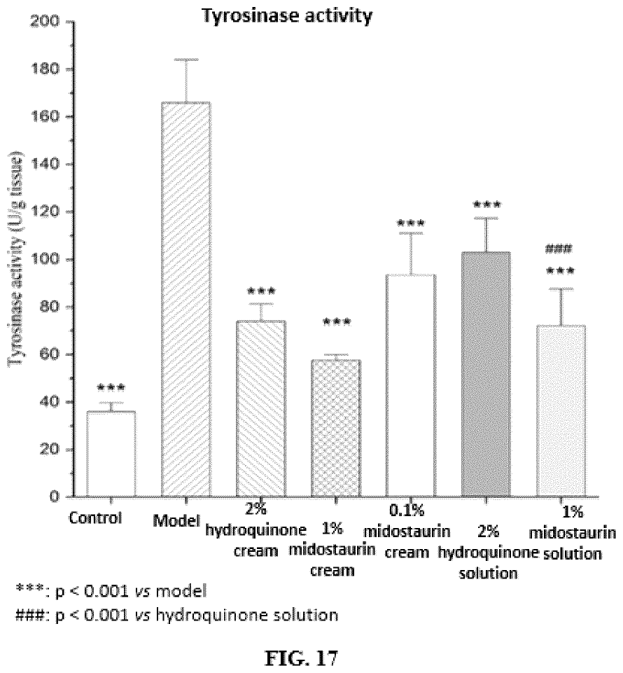

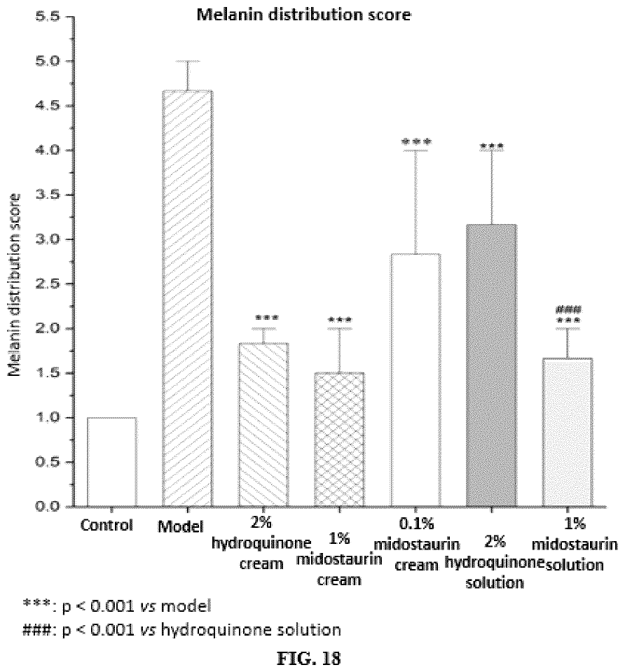

FIG. 17 shows the results for tyrosinase activity for seven different groups, control group (no UV irritation, as shown in FIGS. 15B-C), model group (UV irritation followed by no active treatment), 2% hydroquinone cream, 2% hydroquinone solution, 1% midostaurin cream, 0.1% midostaurin cream, and 1% midostaurin solution treatment groups. FIG. 18 shows the melanin distribution score for seven different groups, control group (no UV irritation, as shown in FIGS. 15B-C), model group (UV irritation followed by no treatment), 2% hydroquinone cream, 2% hydroquinone solution, 1% midostaurin cream, 0.1% midostaurin cream, and 1% midostaurin solution treatment groups.

Comparing to the skin color of model group (site 1), the 5 sites with drugs treatment were lighter and site 3 (1% midostaurin cream) showed the lightest color. Melanin particle distribution scores of 2% hydroquinone cream, 1.0% midostaurin solution, and midostaurin creams (0.1%, 1.0%) reduced significantly compared to that of the model group, and showed a dose dependency in midostaurin cream. Based on the results of this animal study, 1% midostaurin cream showed the best efficacy among all treatment groups, including the 2% hydroquinone cream that was used as a positive control.

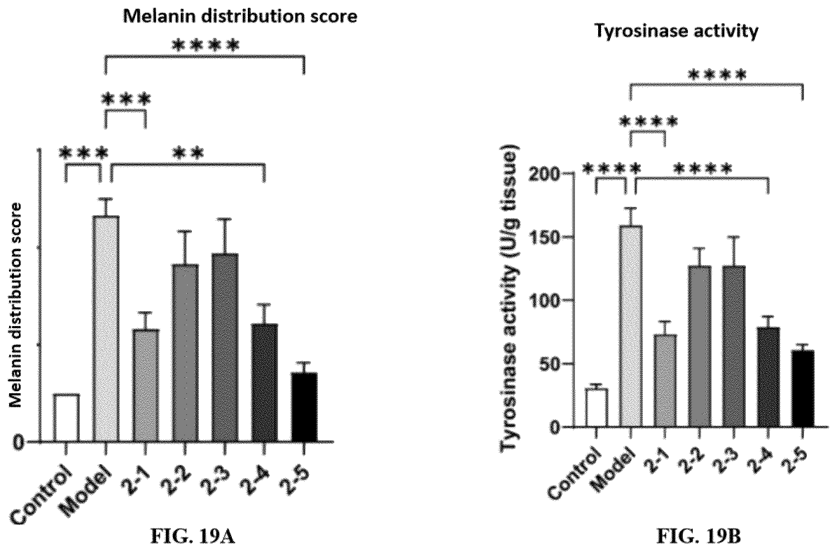

Another efficacy study using the same animal model and protocol described above was conducted to confirm the dose dependency in midostaurin cream and that the high dose (1.5% midostaurin cream) is more effective than 2% hydroquinone. A control group (no UV irritation) and six treatment groups, including model group (UV irritation followed by no treatment), 2% hydroquinone positive control (referred to as “2-1” in FIGS. 19A-19B), 0.01% midostaurin cream (referred to as “2-2” in FIGS. 19A-19B), 0.05% midostaurin cream (referred to as “2-3” in FIGS. 19A-19B), 0.25% midostaurin cream (referred to as “2-4” in FIGS. 19A-19B), 1.5% midostaurin cream (referred to as “2-5” in FIGS. 19A-19B) were tested. As shown in FIGS. 19A-19B, both hydroquinone and midostaurin cream reduced the tyrosinase activity and melanin distribution relative to the model group, and a positive correlation between the dose and the reduction in tyrosinase activity and melanin distribution was observed in midostaurin cream.

The formulation stability such as assay and impurities of the midostaurin topical formulations described herein can be tested, for example, using the HPLC method described in Table A-1 and Table B-2, at different time points under various conditions, for example, after 1 month, 3 months, 6 months, 12 months, 24 months, and/or 36 months of storage at refrigerated conditions, ambient conditions, and/or accelerated conditions.

### Example H

### Skin Irritation Test

A skin irritation study was performed to evaluate the local skin irritation after repeated skin application of midostaurin cream for 28 days using New Zealand rabbits as a model. This trial consists of 2 groups, the blank control group (saline) and the treatment group. The rabbits in the treatment group were given 1% midostaurin cream, positive control (2% hydroquinone cream), or blank base of midostaurin cream based on the amount of cream. The treatments were given to the back skin of each rabbit at a dose of 0.2 g/rabbit twice a day for 28 consecutive days. The general condition of the rabbit and the condition of the administration site was observed daily. The skin irritation score and irritation intensity were evaluated at the following time points; before the first dose of each day, 1 hour after the last dose of each day, and 1, 24, 48, and 72 hours after the last dose of the last day. A widely recognized and standardized skin irritation scoring system, set forth by the Chinese National Medical Products and Administration, was adopted for skin irritation evaluation. More specifically, skin irritation was evaluated based on 1) erythema and eschar formation, and 2) edema formation, according to the standards shown in Table H-1 and Table H-2.

TABLE H-1
Standard for skin irritation response score.

| Reaction | Score |
|---|---|
| Erythema and eschar formation | |
| No erythema | 0 |
| Slight erythema (barely visible) | 1 |
| Moderate erythema (obviously visible) | 2 |
| Severe erythema | 3 |
| Purplish red erythema to slight eschar formation | 4 |
| Edema formation | |
| No edema | 0 |
| Slight edema (barely visible) | 1 |
| Moderate edema (obvious skin bulge) | 2 |
| Severe edema (skin bulge of ~1 mm with clear outline) | 3 |
| Very severe edema (skin bulge of ~≥1 mm and extending | 4 |
| beyond the area of exposure) | |

TABLE H-2
Standard for skin irritation level.

| Irritation level | Score |
|---|---|
| No irritation | 0-0.49 |
| Slight irritation | 0.5-2.99 |
| Moderate irritation | 3.0-5.99 |
| Severe irritation | 6.0-8.0 |

The irritation score results are shown in Table H-3. The results suggest that under the conditions of this test, New Zealand white rabbits were transdermally treated with 0.2 g/rabbit twice a day for 28 consecutive days, 1% midostaurin cream shows mild irritation, which is much better than the moderate irritation of commercial hydroquinone cream.

TABLE H-3
Skin irritation score of different test groups.

| | | | H-3. blank | |
|---|---|---|---|---|
| | | H-2. 1% | base of the | H-4. Blank |
| | H-1. Positive control | midostaurin | cream used | control |
| Test group | (2% hydroquinone cream) | cream | in H-2. | (saline) |
| Irritation score | 5.08 | 1.14 | 0.71 | 0 |
| Level of irritation | Moderate | Mild | Mild | Non-irritating |

### Example I

### Phototoxicity Test

Phototoxicity of midostaurin cream was tested using British guinea pigs as a model. Male guinea pigs were randomly divided into groups and given a single skin application of physiological saline (negative control), 8-MOP (positive control), 1.0% midostaurin cream, and midostaurin cream blank matrix. The dose given is 0.2 mL/site and the administration site was fixed after the treatment was applied. After 0.5 hours, the administration site was irradiated by the sunlight simulator of Dr. Hönle AG (SOL500) to reach the specified irradiation amount of 10±1 J/cm². The irradiance was measured with a UV radiometer equipped with type UVA FO from Dr. Hönle AG. The first day of dosing is designated as day 1 of the trial (Day 1). After the irradiation, the skin irritation of the administration site was observed and scored at 1+0.5 hours, 24 hours, 48 hours and 72 hours, respectively. No animals were dying or found dead during the experiment. No clinical observations or weight changes related to the midostaurin cream were seen in the trial. Edema and erythema can only be seen at the site of the positive control substance (8-MOP, UV irradiation), but not at the site of the positive control substance without UV irradiation, the two control substances with UV irradiation; midostaurin blank matrix and negative control substance (saline), and the 1% midostaurin cream. Thus, no significant difference in skin irritation score was found between the skin sites treated with saline or midostaurin cream once on the skin after a 3-day observation period in guinea pigs. Therefore, under the conditions of this test, 1% midostaurin cream has no phototoxicity.

### Example J

### Pharmacokinetic Study

A pharmacokinetic study was conducted to evaluate the transdermal absorption of midostaurin in Bama pigs after single and 7 consecutive days of transdermal administration of midostaurin cream.

Single transdermal administration; After shaving the back of the Bama pig, transdermally administer a single dose of 42 mg of 1% midostaurin cream (approximately 5 cm×5 cm area) near the spine. Blood was collected before the dose and 30 mins, 1 h, 2 h, 4 h, 6 h, 8 h, 24 h, 48 h, 72 h and 96 h after the dose. Skin tissue at the administration site was collected after the last blood collection. LC-MS/MS methods were used to detect midostaurin level in plasma samples and skin tissue samples.

Seven-day consecutive transdermal administration: After shaving the back of the Bama pig, transdermally administer 42 mg of 1% midostaurin cream (approximately 5 cm×5 cm area) near the spine twice daily for 7 days, two doses were administered 8 hours apart. Blood was collected before the first dose and 30 mins, 1 h, 2 h, 4 h, 6 h, 8 h, 24 h after the first dose, 30 mins after the dose on Day 3, and 30 mins, 1 h, 2 h, 4 h, 6 h, 8 h, 24 h, 48 h, 72 h and 96 h after the last dose. Skin tissue at the administration site was collected after the last blood collection. LC-MS/MS methods were used to detect midostaurin level in plasma samples and skin tissue samples.

The results suggest that after a single transdermal administration of 1% midostaurin cream to Bama pigs, the concentrations of midostaurin in the plasma were below the lower limit of quantification or not detected, and the concentrations of midostaurin in the stratum corneum, epidermis and dermis were 34.1±13.8 μg/g, 12.1±3.38 μg/g, and 0.280±0.141 μg/g, respectively. After Bama pigs were transdermally administered 1% midostaurin cream for 7 consecutive days, the concentration of midostaurin in the plasma was below the lower limit of quantification or was not detected, and the concentrations in the stratum corneum, epidermis and dermis were 268±124 μg/g, 113±63.3 μg/g, and 3.61±1.53 μg/g, respectively.

Thus, the distribution of midostaurin decreased sequentially in the stratum corneum, epidermis and dermis of Bama pigs after transdermal administration of 1% midostaurin cream, 42 mg each time, for a single time or for 7 consecutive days (twice a day). The concentrations of midostaurin in the blood were extremely low, below the lower limit of quantification or not detected, which suggested that the systemic exposure of the midostaurin was extremely low.

It should be appreciated that the above-listed components and methods should be taken as merely exemplary, and not limiting, of the types of components and methods that are encompassed in the present disclosure.

1. A method of treating a skin-related disease or disorder, the method comprising topically applying a multi-targeting protein kinase inhibitor or a salt thereof to a skin of a subject, wherein the skin is associated with a skin-related disease or disorder.

2. The method of claim 1, wherein the skin-related disease or disorder is skin pigmentation disorder.

3. (canceled)

4. (canceled)

5. The method of claim 1, wherein the multi-targeting protein kinase inhibitor is midostaurin or a pharmaceutically acceptable salt thereof.

6. The method of claim 1, wherein the subject has a skin hyperpigmentation condition.

7. The method of claim 6, wherein the skin hyperpigmentation condition comprises a sunspot or an age spot.

8. The method of claim 6, wherein the skin hyperpigmentation condition comprises melasma, post-inflammatory hyperpigmentation, posttraumatic pigmentation, discoid lupus erythematous, freckles, plaques gravidarum, nevus of Ota, or aging pigmentation.

9. The method of claim 5 wherein the method skin-related disease or disorder is melasma.

10. The method of claim 9, wherein the skin is on the subject's face.

11. The method of claim 9, wherein the melasma is chloasma.

12. The method of claim 11, wherein the melasma is caused by or exacerbated by one or more of: birth control pills, pregnancy, hormone therapy, stress, thyroid disease, sunlight exposure, inflammation, familial predisposition, or free radicals.

13. The method of claim 1, wherein the treating comprises reducing the size of abnormal skin pigmentation, reducing the intensity of abnormal skin pigmentation, and/or eliminating the abnormal skin pigmentation associated with the melasma.

14. The method of claim 1, wherein the treating comprises reducing melanin amount in an area of the skin by at least 25% compared to a similar area of skin in the same subject without the treatment.

15. The method of claim 1, wherein the treating comprises reducing melanin amount in an area of the skin by at least 1.0 according to Melanin Distribution Score compared to a similar area of skin in the same subject without the treatment.

16. The method of claim 1, wherein the treating comprises reducing melanin amount in an area of the skin by at least 1.0 according to Fontana-Masson staining scoring standard compared to a similar area of skin in the same subject without the treatment.

17. The method of claim 1, wherein the method does not create moderate or severe skin irritation in the subject.

18. The method of claim 1, comprising topically applying a pharmaceutical composition, wherein the pharmaceutical composition comprises midostaurin or a pharmaceutically acceptable salt thereof.

19. The method of claim 1, wherein a therapeutically effective amount of the multi-targeting protein kinase inhibitor or a salt thereof is a dose that is 80% less than that for hydroquinone.

20. A topical pharmaceutical composition comprising
a) a multi-targeting protein kinase inhibitor (optionally midostaurin) or a pharmaceutically acceptable salt thereof in an amount of 0.001% to about 20% wt;
b) a carrier vehicle; and
c) an excipient;
wherein the topical pharmaceutical composition is formulated for topical administration.

21-31. (canceled)

32. The topical pharmaceutical composition of claim 20, wherein the excipient comprises a surfactant, a penetration enhancer, an antioxidant, a sun-screening agent, a viscosity modifier, a pH stabilizer, or a moisturizer, or any combination thereof.

33-64. (canceled)

65. The topical pharmaceutical composition of claim 20, wherein the topical pharmaceutical composition is in a form selected from an ointment, a gel, a cream, a lotion, a solution, an emulsion, a paste, a patch, a wipe, a swab, and a pad.

66-72. (canceled)

73. The topical pharmaceutical composition of claim 20, wherein the topical pharmaceutical composition comprises;
midostaurin or a pharmaceutically acceptable salt thereof in an amount of about 0.01% to about 5% wt;
a carrier vehicle in an amount of about 30% to about 70% wt;
a surfactant in an amount of about 5% to about 50% wt;
optionally a moisturizer in an amount of about 5% to about 30% wt;
optionally a penetration enhancer in an amount of about 1% to about 40% wt;
optionally a viscosity modifier in an amount of about 0.01% to about 5% wt; and
an antioxidant in an amount of about 0.001% to 5% wt.

74-92. (canceled)

93. A method of reducing tyrosinase activity, the method comprising topically applying a topical pharmaceutical composition to a skin of a subject in need thereof, wherein the topical pharmaceutical composition comprises midostaurin or a pharmaceutically acceptable salt thereof.

94-96. (canceled)

97. A kit comprising a package enclosing the topical pharmaceutical composition of claim 20.

98. The method of claim 18, wherein the pharmaceutical composition is applied at a dose from about 0.01 mg to about 20 mg per day.

99. The method of claim 18, wherein the pharmaceutical composition is applied at a dose from about 0.1 mg to about 5 mg per day.

100. The method of claim 18, wherein the pharmaceutical composition is applied at a dose of about 0.1 mg per day, about 0.2 mg per day, about 0.3 mg per day, about 0.4 mg per day, about 0.5 mg per day, about 0.6 mg per day, about 0.7 mg per day, about 0.8 mg per day, about 0.9 mg per day, about 1 mg per day, about 1.5 mg per day, about 2 mg per day, about 2.5 mg per day, about 3 mg per day, about 3.5 mg per day, about 4 mg per day, about 4.5 mg per day, or about 5 mg per day.

101. The method of claim 18, wherein the pharmaceutical composition is applied at a dose that is 80% less than that for 2% hydroquinone cream, 70% less than that for 2% hydroquinone cream, or 50% less than that for 2% hydroquinone cream.

102. The method of claim 18, wherein the pharmaceutical composition is applied once a day.

103. The method of claim 18, wherein the pharmaceutical composition is applied twice a day.

104. The method of claim 18, wherein the pharmaceutical composition is applied once a day or twice a day for at least one week.
